(19) United States
(12) Patent Application Publication
(10) Pub. No.: US 2008/0207489 A1
(43) Pub. Date: Aug. 28, 2008

(54) Use of CCN5 for treatment of smooth muscle proliferation disorders

(76) Inventors: John Castellot, Newton, MA (US); Mark R. Gray, West Roxbury, MA (US); Amy R. Simon, Newton, MA (US)

Correspondence Address: LAWSON & WEITZEN, LLP, 88 BLACK FALCON AVE, SUITE 345, BOSTON, MA, 02210, US

(21) Appl. No.: 12/070,814

(22) Filed: Feb. 21, 2008

Related U.S. Application Data
(60) Provisional application No. 60/961,859, filed on Jul. 25, 2007.
(60) Provisional application No. 60/890,865, filed on Feb. 21, 2007.

(51) Int. Cl. A61K 38/02; A61K 48/00; A61P 35/00; A61P 21/00

(52) U.S. Cl. 514/2; 514/44

(57) ABSTRACT

The invention provides methods for treating a smooth muscle cell disorder such as asthma, vascular injury, persistent pulmonary hypertension in the newborn, pulmonary hypertension in adults, megaureter, pyloric stenosis, uterine fibroids, lymphangioleiomyomatosis (LAM), cervical incompetence, or cancer, and methods for assessing smooth muscle tissue remodeling in a mammal by expressing CCN5 in a smooth muscle cell of the mammal, or by contacting the tissue directly with CCN5.

### RELATED APPLICATIONS

This application claims the benefit of U.S. provisional applications serial Nos. 60/890,865 filed Feb. 21, 2007 and 60/961,859 filed Jul. 25, 2007 in the U.S. Patent and Trademark office, both of which are hereby incorporated by reference herein in their entireties.

### GOVERNMENT SUPPORT

The invention was supported in part by NIH Grants HL049973 and HD046251. The government has certain rights in the invention.

### TECHNICAL FIELD

Methods are provided for treating a subject having a smooth muscle cell proliferation-based disorder such as asthma, vascular injury, persistent pulmonary hypertension in a newborn, pulmonary hypertension in adults, megaureter, pyloric stenosis, uterine fibroids, lymphangioleiomyomatosis (LAM), cervical incompetence, or cancer, by providing CCN5 to the subject, and methods are provided for assessing smooth muscle tissue remodeling in a mammal.

### BACKGROUND

Abnormal smooth muscle cell proliferation and migration is involved in many common and medically important diseases. Asthma, for example, is one of the fastest-growing diagnosed conditions in the United States, asthma afflicts more than 17 million Americans, with approximately one-third of all cases refractory to current therapies. It is characterized by airway narrowing, inflammation, and mucus hyper-secretion. Although these pathophysiologic changes previously were thought to be reversible, there is now increasing evidence that in the airway of some patients, structural changes contribute to chronic obstruction and airway hyper-responsiveness. These structural changes, referred to as airway wall remodeling (AWR), include airway smooth muscle (ASM) hypertrophy and hyperplasia, myofibroblast hyperplasia, epithelial metaplasia, vascular dilatation, and fibrosis accompanied by excessive extracellular matrix (ECM) deposition. However, little information is available on the biochemical and molecular biologic mechanisms underlying AWR, particularly on the role of increased ASM hyperplasia in the pathogenesis of asthma.

The basic functions of ASM must be accomplished in an environment of continuous changes in stretch, tensile forces, and exposure to environmental pathogens, allergens, and irritants. Allergic asthma is characterized by infiltration of ASM by mast cells and other inflammatory cells, including lymphocytes, neutrophils, and monocyte/macrophages (Travis et al., 2005; Brightling et al., 2002; Busse et al., 2001). It is thought that close contact between ASM and infiltrating cells is a major contributing factor in inflammation and AWR through the participation of cytokines and other inflammatory agents produced by the infiltrating cells (Brightling et al., 2002). The result of this process is hyper-proliferation of smooth muscle cells (SMC) and concomitant thickening of the airway wall and the deleterious sequelae of decreased pulmonary function.

Despite the importance of ASM hyperplasia in asthma, the processes of proliferation and motility in ASM have been studied much less in airway than in vascular smooth muscle (VSM). Studies have focused primarily on mitogens or mitogenic signaling pathways (Homer and Elias, 2000). Mediators of ASM proliferation in vitro include cytokines (IL-1, IL-6, IL-13), growth factors (PDGF, EGF), reactive oxygen species (ROS), and inflammatory mediators (histamine; Busse et al., 1999; Elias et al., 2003). Although several well-characterized inhibitors of VSM proliferation have been reported (Gray and Castellot, 2004), none of them have been examined in ASM.

In addition to hyper-proliferation, increased ASM motility and major ECM modifications occur during the pathogenesis of asthma. These processes involve matrix metalloproteinases (MMPs), including MMP-2, -8, -9, -12, and MT-1 (Maisi et al., 2002). Expression of MMP-9 and MMP-12 is increased from normal levels, after exposure to the stimulating allergen and during the course of the disease, causing ECM destruction and abnormal ECM repair (Cataldo et al., 2003). Levels of tissue inhibitor of metalloproteinase-1 (TIMP-1) rise during chronic asthma, eventually blocking the activity of MMPs, resulting in excessive collagen deposition (Corbel et al., 2003). Rigorous examination of specific MMPs and TIMPs in the pathogenesis of asthma using gain- or loss-of-function studies has not been accomplished.

Lymphangioleiomyomatosis (LAM) is a progressive lung disease that affects women and is found in populations of all races, usually during their childbearing years. Symptoms include collapsed lung, fluid in the lungs, shortness of breath, fatigue, cough, and chest pain. LAM is often misdiagnosed as asthma, emphysema, or pulmonary bronchitis. Scientists estimate that as many as 250,000 to 300,000 LAM patients are misdiagnosed or undiagnosed.

The life-long loss of quality of life that results from the airway remodeling that occurs in chronic asthma afflicts millions of Americans, yet no therapy currently exists for this important part of the pathogenesis of asthma. In fact, far less is known about the basic cellular and molecular mechanisms regulating airway smooth muscle function than is known about their vascular counterparts. The need for breaking new ground both therapeutically and by increasing our understanding of airway pathobiology is therefore critical.

### SUMMARY

An embodiment of the invention herein provides a method for treating a smooth muscle proliferation-based disorder in a mammalian subject, the method including expressing CCN5 in or administering CCN5 protein to smooth muscle cells, the disorder being at least one selected from the group of: asthma, vascular injury, persistent pulmonary hypertension in the newborn (PPNH), pulmonary hypertension in adults, megaureter, pyloric stenosis, uterine fibroids, lymphangioleiomyomatosis (LAM), cervical incompetence, and cancer.

In a related embodiment of the method, the disorder is asthma, and the CCN5 is expressed in airway smooth muscle (ASM) cells by contacting ASM with an expression vector encoding CCN5, the vector selected from a virus vector and a nucleic acid vector, or by contacting ASM with recombinantly produced CCN5 protein. In an alternative embodiment, expression of CCN5 is mediated by a small molecule or other agent that causes CCN5 to be over-expressed. The delivery route for the vector or the protein is via inhalation. Similarly, the delivery route for the small molecule or other agent is via inhalation.

Another embodiment of the invention herein provides a method for evaluating airway remodeling including: modulating the expression of CCN5 in ASM in a mammal having symptoms of chronic asthma; and assessing the morphology of ASM in comparison to a control having the symptoms and otherwise identical to the mammal and not modulated in expression of CCN5.

Another embodiment of the invention herein provides a method for treating a smooth muscle disorder in a mammal including reducing the expression or activity in the smooth muscle tissue of one or more genes that are suppressed by expression of CCN5. In a related embodiment, the one or more genes is selected the group consisting of LILRA1, DEFB103A, LOC387643, LY6K and OR4X2. In a related embodiment, expression of one or more genes selected from the group consisting of LILRA1, DEFB103A, LOC387643, LY6K and OR4X2 is reduced in a smooth muscle cell by expressing an antisense RNA, ribozyme or siRNA that targets RNA encoding one or more genes selected from the group consisting of LILRA1, DEFB103A, LOC387643, LY6K and OR4X2. In yet another related embodiment, expression or activity of one or more genes selected from the group consisting of LILRA1, DEFB103A, LOC387643, LY6K and OR4X2 is mediated by a small molecule or other agent that reduces the expression or activity of one or more genes selected from the group consisting of LILRA1, DEFB103A, LOC387643, LY6K and OR4X2. A related embodiment of the method involving antisense RNA, ribozyme or siRNA further includes delivering a nucleic acid expression vector by inhalation, and a related embodiment of the method involving the small molecule or other agent is delivered via inhalation. In certain embodiments of the methods, reducing expression further includes contacting a smooth muscle cell with an antisense oligonucleotide, ribozyme, or siRNA that targets one or more genes selected the group consisting of LILRA1, DEFB103A, LOC387643, LY6K and OR4X2, and contacting the cell with the antisense oligonucleotide, ribozyme, or siRNA further includes delivering by inhalation.

An embodiment of method provided herein involving vascular injury includes the vascular injury that is at least one condition selected from the group of tensile strain, shear strain, vessel rupture, intimal rupture, penetrating trauma, blunt trauma, transection, contusion, laceration, arteriovenous (AV) fistula formation, vessel spasm, external compression, mural contusion, thrombosis, and aneurysm formation. The method further includes the vascular injury which is stenosis or restenosis associated with at least one of wire injury, ligation injury, arteriovenous shunt, coronary artery bupass graft, endarterectomy, hypertension and balloon angioplasty. In a related embodiment, the method further includes, following expressing CCN5 in smooth muscle cells, observing substantial inhibition of neointimal lesion formation in comparison to a vascular injury otherwise identical not expressing CCN5. In a related alternative or additional embodiment, the method further includes, following expressing CCN5 in smooth muscle cells, observing substantial inhibition of at least one cell parameter selected from the group of proliferation, motility and matrix metalloprotease production.

In a related embodiment of the method in which the disorder is vascular injury, the CCN5 is expressed in vascular smooth muscle cells by contacting vascular smooth muscle cells with an expression vector encoding CCN5, the vector selected from a virus vector and a nucleic acid vector, or contacting the cells with recombinantly produced CCN5 protein. In yet another related embodiment of the method, contacting the cells with recombinantly produced CCN5 protein is delivery by catheter or by coated stent. In an embodiment of the method, the vascular injury arises from atherosclerosis.

In a related embodiment of the method in which the disorder is a cancer, the CCN5 is expressed in vascular smooth muscle cells of a tumor by contacting the tumor with an expression vector encoding CCN5, the vector selected from a virus vector and a nucleic acid vector, or contacting the cells with recombinantly produced CCN5 protein. In yet another related embodiment of the method, contacting the cells with recombinantly produced CCN5 protein is delivery by catheter or direct surgical implant. A related embodiment of the method in which the smooth muscle disorder is cancer, the cancer is a tumor. In a related embodiments, the tumor is at least one selected from the group of myolipoma, cystic liver tumor, hepatic tumor, angiolipoleiomyoma, leiomyoma, and leiomyosarcoma.

### BRIEF DESCRIPTION OF THE DRAWINGS

FIG. 1 is a set of photomicrographs that show that CCN5 is greatly reduced in smooth muscle cells in the airways of asthmatic mice. Using a mouse model (Johnson et al., 2004), either a buffer containing house dust mite extract (HDM; right panel), or a control buffer (phosphate-buffered saline (PBS); left panel) was applied intra-nasally 5 days per week for 7 weeks (right panel). After a 7-week treatment, the animals were sacrificed and lung tissue was analyzed by immunohistochemistry using a highly specific anti-CCN5 antibody. CCN5 expression appeared as brown staining (Lake and Castellot, 2004). Airway smooth muscle cells are indicated by an * in each panel. The data show that HDM treatment reduces areas of brown stain or CCN5.

FIG. 2 is a bar graph that shows that mitogenic stimulation of human airway smooth muscle cells (hASM) causes a rapid reduction in CCN5 mRNA (right lane). hASM were sparsely plated into 35 mm culture dishes and then subjected to low-serum (0.1%) medium for 3 days to induce growth arrest. A small volume of medium without serum and with or without 15 ng/ml platelet-derived growth factor (PDGF) was added to the cells. After two hours, the cells were lysed, and the lysates were collected and processed for quantitative PCR to measure CCN5 mRNA levels. Data show that CCN5 mRNA is reduced about 50% by PDGF treatment

FIG. 3 is a set of photomicrographs that shows that CCN5 suppresses restenosis in the carotid ligation model of vascular injury. The carotid ligation method of Kumar et al. (1997) was used to induce SM hyperplasia in mice. The left carotid was ligated and clamped for 25 minutes, during which time either AdCCN5, AdGFP, or buffer was injected into the ligated region. After 25 minutes, the clamp was released. Animals were sacrificed at various time points after the injury; the data shown were obtained from animals 14 days after the injury. IEL=internal elastic lamina. The data show substantial restenosis (mid-panel) suppressed by AdCCN5 injection (right panel).

FIG. 4 is a bar graph that shows data from quantitative morphometry of CCN5 effect on SM hyperplasia. Animals were sacrificed 14 days following left carotid ligation and adenovirus treatment, and sections were taken and perfusion-fixed with 4% paraformaldehyde.

Injured left arteries (bars A-E) and uninjured right carotid arteries (right) were removed and processed for histologic sectioning. Sections located at approximately 1.2 mm from ligation (mid-point of the injured region) were stained with Verhoeff-VanGieson stain to visualize elastic fibers. Morphometric software (Spot Software, Diagnostic Instruments, Inc.) was used to determine the neointimal area, defined as the area between the IEL and the border of the lumen. Bars A-C, data from each of the three individual mice injected with AdGFP; bars D-E, 2 separate mice injected with AdCCN5. The data shown an unexpected extent of decrease (5 to 10-fold) in neointimal area in AdCCN5 treated arteries.

FIG. 5 is a set of bar graphs showing intima areas of vascular media following carotid ligation. Panel A shows vascular intima area (defined as area between internal elastic lamina and lumen). FIG. 5 Panel B shows vascular media area (defined as area between external and internal elastic laminae) following carotid ligation. n=number of animals examined. Areas were found to be increased compared to mock controls, and increased as a function of time from day 2.

FIG. 6 is a set of photomicrographs showing CCN5 protein expression following carotid ligation. FIG. 6 Panel A shows contralateral uninjured artery. FIG. 6 Panel B shows mock injury. FIG. 6 Panel C shows negative control (PBS). Panels D-K are ligated arteries at indicated day following procedure. FIG. 6 Panel D shows day 2 (200×). FIG. 6 Panel E shows day 2 (630×). FIG. 6 Panel F shows day 5. FIG. 6 Panel G shows day 8. FIG. 6 Panel H shows day 11. FIG. 6 Panel I shows day 14 (2 weeks) 200×. FIG. 6 Panel J shows day 14 (2 weeks) 630×. FIG. 6 Panel K shows day 18. FIG. 6 Panel L shows day 21 (3 weeks). FIG. 6 Panel M shows day 28 (4 weeks). FIG. 6 Panel N shows day 56 (8 weeks). FIG. 6 Panel O shows day 84 (12 weeks). Brown staining indicates CCN5 protein expression. Nuclei were counter-stained blue with hematoxylin. All negative controls completely lacked brown staining. FIG. 6 Panels A-D, F-H, and K-O are enlarged 200×. Panels E and I are enlarged 630×. Data show that CCN5 expression increased compared to controls shown in Panels A, B and C.

FIG. 7 is a set of photomicrographs showing AdGFP transfection 2 days following carotid ligation injury. FIG. 7 Panel A shows a control which is a contralateral uninjured artery (and did not receive virus). Panel B shows injured artery treated with AdGFP. The data show little effect of AdGFP compared to the control.

FIG. 8 is a set of photomicrographs showing that AdCCN5 treatment limits response to carotid ligation injury. The method involved Verhoeff-Van Gieson stained sections of ligated arteries that had received each of the indicated treatments. FIG. 8 Panel A, saline; FIG. 8 Panel B, AdGFP; and FIG. 8 Panel C, AdCCN5. The CCN5-treated artery in panel C showed much less response to injury than controls.

FIG. 9 is a bar graph showing that AdCCN5 treatment limits the increased neointima and media areas following carotid ligation. Vascular neointima area is defined as area between internal elastic lamina (IEL) and lumen. Vascular media area is defined as the area between external and IEL following carotid ligation. n=number of animals examined, * indicates statistical significance (p<0.05). The data show that CCN5 treatment substantially reduced the area increases observed in controls especially in the neointima.

FIG. 10 is a set of photomicrographs that shows that CCN5 expression remains low in media 14 days following injury in treated animals. Brown staining indicates CCN5 protein expression. Nuclei were counter-stained blue with hematoxylin. FIG. 10 Panel A, saline; FIG. 10 Panel B, AdGFP; FIG. 10 Panel C, AdCCN5; FIG. 10 Panel D, negative control (PBS). Result obtained in all negative controls indicated complete lack of brown staining.

FIG. 11 is a set of photomicrographs that shows that medial CCN5 expression is reduced in cycling and non-cycling cells. FIG. 11 Panel A, CCN5 expression (brown staining) 8 days following carotid ligation; FIG. 11 Panel B. negative control for anti-CCN5 staining (PBS); FIG. 11 Panel C, BrdU incorporation (black staining) 8 days following carotid ligation; and FIG. 11 Panel D, negative control for anti-BrdU staining (PBS). Nuclei were counter-stained blue with hematoxylin.

FIG. 12 is a set of photomicrographs that shows that AdCCN5 limits BrdU incorporation in media. FIG. 12 Panel A, saline; FIG. 12 Panel B, AdGFP; FIG. 12 Panel C, AdCCN5; and FIG. 12 Panel D, negative control (PBS). Black staining indicates BrdU incorporation. Nuclei were counter-stained blue with hematoxylin. All negative controls completely lacked black staining. The CCN5 and enhanced green fluorescent protein (GFP) adenovirus used in these experiments were previously described (Lake et al., 2003; Mason et al., 2004). The CCN5 adenovirus (AdCCN5) expressed both GFP and CCN5 tagged by a nine amino acid HA epitope on the C-terminus. These two genes were under the control of separate CMV promoters. The control virus (AdGFP) expressed only GFP. The data show that BrdU incorporation was decreased in CCN5-treated tissue (FIG. 12 Panel C) compared to the control (FIG. 12 Panel B).

FIG. 13 is a set of photographs that shows tumors formed from CCN5 expressing cells (tumors on the right in each photograph) and tumors formed from GFP expressing cells (tumors on the left in each photograph) in two different mice (FIG. 13 Panel A and FIG. 13 Panel B respectively). These photographs show that tumors formed from CCN5 expressing cells appear grossly smaller and less vascular then tumors formed from GFP expressing cells. Tumors formed from CCN5 expressing cells form with a lower mass and smaller dimensions then tumors formed from GFP expressing cells as demonstrated by quantitation of dimensions of the tumors (see Table 1).

FIG. 14 is a set of photomicrographs showing CCN5 expression throughout mouse development. Mouse embryonic and fetal sagittal sections from Panels: A. E9, B. E10, C. E11, D. E12, E. GD14 and F. GD16 developing mice, respectively. Negative control (Panel F right) was performed using pooled rabbit IgG applied in place of primary antibody. Abbreviations: Bl=Bladder; Br=Brain; He=Heart; Int=Intestine; K=Kidney; Li=Liver; Lu=Lung; and To=Tongue. FIG. 14 Panels A-E, CCN2 left, CCN5 right; and Panel F, CCN2 left, CCN5 middle, control right. Scale Bars=1 mm. Scale Bars and labels on CCN5 panels also apply to CCN2 panels. CCN5 and CCN2 immunoreactivity is shown by extent of brown staining. Nuclei were stained by hematoxylin (blue). Tissue sections treated with pooled rabbit IgG gave no detectable staining (FIG. 14, Panel F).

FIG. 15 is a set of photomicrographs showing CCN5 expression during cardiovascular development. FIG. 15, Panel A is GD16 lung with artery and vein; FIG. 15 Panel B is E12 mouse heart; FIG. 15 Panel C is GD14 mouse heart; FIG. 15 Panel D is GD14 mouse heart; FIG. 15 Panel E is GD16 mouse heart; FIG. 15 Panel F is GD14 mouse umbilical vessels; FIG. 15 Panel G is Human 5 month myocardium and coronary vessels; FIG. 15 Panel H is Human 5 month fetal blood vessel; and FIG. 15 Panel I is Human 5 month fetal umbilical cord. Negative control (FIG. 15 Panel F right) was performed using pooled rabbit IgG applied in place of primary antibody. Abbreviations: A=Artery; Ao=Aorta; CV=coronary vessels; PA=Pulmonary Artery; V=Vein: AV [symbol] =atrioventricular septa; ▴=apex; and Δ=Valve. FIG. 15 Panels A-E and G-I, CCN2 left and CCN5 right. FIG. 15 Panel F, CCN2 left, CCN5 middle, and control right. Scale Bars=100 μm. Tissue sections treated with pooled rabbit IgG gave no detectable staining (FIG. 15 Panel F).

FIG. 16 is a set of photomicrographs showing CCN5 expression during lung development. FIG. 16 Panel A is GD14 mouse lung and FIG. 16 Panel B is GD16 mouse lung. Left insets show actively branching bronchioles. Right insets show larger bronchi that are no longer branching. FIG. 16 Panel C is Human 5 month fetal lung. Abbreviations: ep=epithelial cells and me=mesenchymal cells. FIG. 16 Panels A-C, CCN2 left and CCN5 right. Scale Bars=100 μm. Tissue sections treated with pooled rabbit IgG gave no detectable staining.

FIG. 17 is a set of photomicrographs showing CCN5 expression in developing bones and skeletal muscle. FIG. 17 Panel A is GD14 skeletal muscle; FIG. 17 Panel B is GD16 skeletal muscle and bone; FIG. 17 Panel C is GD16 rib; FIG. 17 Panel D is Human 5 month fetal skeletal muscle; and FIG. 17 Panel E is Human 5 month fetal bone. Inset shows a magnified osteoclast. Abbreviations: CZ=Calcification; Zone HC=Hypertrophic Chondrocytes; MyoJxn=Myotendinous Junctions; PZ=Proliferating Zone; RZ=Resting Zones; SkMusc=Skeletal Muscle; OCL=osteoclast; and OCT=osteocyte. FIG. 17 Panels A-E CCN2 left and CCN5 right. Scale Bars=100 μm. Tissue sections treated with pooled rabbit IgG gave no detectable staining.

FIG. 18 is a set of photomicrographs showing CCN5 expression during intestinal development. FIG. 18 Panel A is GD16 mouse liver; FIG. 18 Panel B is E12 mouse intestine; FIG. 18 Panel C is GD14 mouse intestine; FIG. 18 Panel D is GD16 mouse intestine; FIG. 18 Panel E is Human 4 month fetal liver; FIG. 18 Panel F is Human 5 month fetal liver; FIG. 18 Panel G is Human 6 month fetal esophagus; FIG. 18 Panel H is Human 5 month fetal stomach; FIG. 18 Panel I is Human 5 month fetal intestine; FIG. 18 Panel J is Human 5 month fetal gallbladder; FIG. 18 Panel K is Human 5 month fetal colon; and FIG. 18 Panel L is Human 5 month fetal rectum. Abbreviations: EP=intestinal epithelium; Hem=hematopoietic stem cells; Hep=hepatocytes; and SM=intestinal smooth muscle. FIG. 18 Panels A-L, CCN2 left and CCN5 right. Scale Bars=50 μm. Tissue sections treated with pooled rabbit IgG gave no detectable staining.

FIG. 19 is a set of photomicrographs showing CCN5 expression in developing urogenital system. FIG. 19 Panel A is E12 mouse kidney; FIG. 19 Panels B-C are GD16 mouse kidney; FIG. 19 Panel D is GD14 mouse bladder; FIG. 19 Panel E is GD16 mouse bladder; FIG. 19 Panel F is GD14 mouse ovary; FIG. 19 Panel G is GD16 mouse ovary and reproductive tract; FIG. 19 Panel H is Human 5 month fetal kidney; FIG. 19 Panel I is Human 5 month placenta; FIG. 19 Panel J is Human 5 month fetal testis; FIG. 19 Panel K is Human 5 month fetal uterus; FIG. 19 Panel L is Human 5 month fetal ovary; FIG. 19 Panel M is Human 5 month fetal fallopian tube; FIG. 19 Panel N is Human 5 month fetal epididymis. Δ▴=sets of mesonephric tubules in which terminal ends alternately express CCN2 or CCN5. Abbreviations: Br=branch point of chorionic villi with absent CCN5 staining; CD=collecting ducts; DT=distal tubules; E=glomerular endothelial cells; GE=germinal epithelium; Le=leydig cells; M=mesangial cells; Oo=oocytes; SM=smooth muscle; Tr=trophoblasts; and Ur=urothelium. FIG. 19 Panels A-G CCN2 left and CCN5 right. Tissue sections treated with pooled rabbit IgG gave no detectable staining.

FIG. 20 is a set of photomicrographs showing CCN5 expression in endocrine organs. FIG. 20 Panel A is Mouse GD16 thyroid gland; FIG. 20 Panel B is Mouse GD16 adrenal gland; FIG. 20 Panel C is Human 5 month fetal thyroid gland. Insets magnified to show nuclear CCN5 staining in adrenal gland cells. FIG. 20 Panel D is Human 5 month fetal adrenal gland. Abbreviations: F=thyroid follicle; ZF=zona fasciculate; and ZG=zona glomerulosa. FIG. 20 Panels A-D, CCN2 left and CCN5 right. Scale Bars=100 μm. Tissue sections treated with pooled rabbit IgG gave no detectable staining.

FIG. 21 is a set of photomicrographs showing CCN5 expression in developing brain, hair, skin, and eyes. FIG. 21 Panel A is E12 mouse brain; FIG. 21 Panel B is GD14 mouse brain; FIG. 21 Panel C is GD16 mouse brain; FIG. 21 Panel D is GD16 spinal cord; FIG. 21 Panel E is GD16 mouse whisker; FIG. 21 Panel F is GD16 mouse skin; FIG. 21 Panel G is GD16 mouse eye; FIG. 21 Panel H is Human 4 month fetal brain; FIG. 21 Panel I is Human 5 month fetal hair follicle; FIG. 21 Panel J is Human 5 month fetal skin; and FIG. 21 Panel K is Human 4 month fetal retina. Abbreviations: Af=afferent nerve fibers; C=choroids; IRS=inner root sheath; ORS=outer root sheath; and PR=photoreceptors. FIG. 21 Panels A-K, CCN2 left and CCN5 right. Scale Bars=100 μm. Tissue sections treated with pooled rabbit IgG gave no detectable staining.

FIG. 22 is a set of photographs of autoradiograph of electrophoresis gels showing that CCN5 mRNA is expressed in all GD14.5 fetal mouse organs. RNA was harvested from pooled GD14.5 C57BL/6 mouse organs, reverse transcribed, and PCR was performed using primers specific to mouse CCN5 or Tbp (loading control) on mRNA treated with (+RT) or without (−RT) reverse transcriptase. (−) no template control (+) plasmid containing mouse CCN5 cDNA in each of lanes (a)-(k) as follows: (a) maternal uterus (b) placenta (c) lung (d) limbs and tails (e) umbilical cord (f) intestine (g) heart (h) liver (i) carcass (j) head (k) maternal aorta.

### DETAILED DESCRIPTION

The invention provides methods of treatment of smooth muscle proliferation-based disorders such as chronic asthma, vascular injury and cancer. The references cited herein show the knowledge in the field and are hereby incorporated herein by reference in their entireties.

The gysteine-rich connective tissue growth factor/nephroblastoma-overexpressed (CCN) family of proteins consists of six highly conserved, cysteine-rich matricellular proteins, secreted proteins that adhere tightly to the cell surface (Brigstock, 2003). CCN proteins regulate cell proliferation, motility, and ECM production in cultured cells. They have been strongly implicated in many normal and pathologic processes, including angiogenesis, implantation and placental development, fibrosis, vascular restenosis, fibroid formation, and chondrodysplasias. With the exception of CCN5 (also known as WISP-2), CCN proteins have a characteristic structure of four polypeptide modules, including Insulin-Like Growth Factor-Binding Protein (IGF-BP), von Willebrand Factor-C (VWC), thrombospondin-1 (TSP1), and carboxy-terminal (CT) domains.

CCN5 is a member of this family of genes (Perbal et al., 2005). The six members of this family are matricellular proteins that have important functions in numerous cell and physiologic processes, including cell motility and proliferation, vascular disease, angiogenesis, embryonic development, wound repair, fibrosis, extracellular matrix biology, and progression of certain cancers. CCN5, also known as WISP-2 (Pennica et al., 1998), rCop-1 (Zhang et al., 1998), COP-1 (Delmolino et al., 2001), HICP (Delmolino et al., 1997), and CTGF-L (Kumar et al., 1997), is highly conserved among vertebrates and is the only CCN protein that lacks the C-terminal domain present in all other CCN proteins (Gray et al., 2005).

CCN5 is the only CCN family member lacking the CT domain (Delmolino, 1997, 2001). Because of this major structural difference, it is here envisioned that many functions of CCN5 would be significantly different from those mediated by the four-domain CCN proteins. It is shown here that CCN5 has functions different from and even opposite to those of other CCN proteins, and that CCN5 is an antagonist (either pharmacologically or biologically) for the four-domain CCN proteins, such as CCN2, also known as CTGF (Connective Tissue Growth Factor).

CCN5 is heparin- and estrogen-induced, and expressed at high levels in quiescent (Gₒ) VSM and at very low levels in proliferating VSM. The CCN5 expression pattern in VSM is characteristic of a growth arrest-specific gene (Delmolino et al., 2001). VSM contacted with an adenovirus vector over-expressing CCN5 (AdCCN5) demonstrates dose-dependent decreases in VSM proliferation rates, motility, and invasiveness, but not apoptosis (Lake et al., 2003). Transfection with small inhibitory RNA (siRNA) molecules specific for CCN5 causes dysregulated VSM proliferation and increased VSM motility (Lake and Castellot, 2003). Surprisingly, CCN5 knock-down with siRNA increases production of MMP-2 (an MMP that is known to be relevant for VSM motility), but not MMP-9. Approximately half the siRNA-treated VSMC demonstrated a drastic loss of actin microfilaments accompanied by a 50% reduction in actin mRNA levels. The cell morphology changed from the typical spindle shape of SM to a more epithelioid shape. In contrast, CCN5 effects on MMP-2 activity and cytoskeletal organization observed in VSM infected with AdCCN5 were observed to be surprisingly substantially opposite in effect. In a rat carotid artery model for balloon angioplasty, observed high levels of CCN5 were observed in the uninjured artery wall. Following balloon injury, VSM actively migrate through the basement membrane of the intimal endothelial cell layer, proliferate, and form a myointimal lesion. The abundant CCN5 protein expression in the uninjured vessel disappeared within two days after balloon injury, a period of time in which the fraction of proliferating VSM is at the highest, and then expression returned at a time point during which VSM hyperplasia had ceased 14 days after injury (Lake et al., 2003).

Aberrant proliferation of smooth muscle cells (SMC) in the artery wall is the hallmark of several pathological states, including restenosis, arteriosclerosis, and persistent pulmonary hypertension of the newborn. SMC hyperplasia is responsible for the failure of a substantial fraction (as high as 30%) of many vascular surgical procedures, including percutaneous transluminal coronary angioplasty (PTCA), coronary artery bypass grafts (CABG), arterio-venous shunts, endarterectomies, and heart transplants. Nearly 2 million vascular procedures are performed each year in the United States, and the high failure rate remains a significant clinical issue.

Heparin inhibits the proliferation and motility of SMC in animal models (Saku et al., 1989; Tan et al., 1991) and in cultured SMC (Charonis et al., 1983, Stearns et al.1997), reviewed in Lyons-Giordano et al., 1987. Mechanistic studies on heparin revealed that greater than 80% of the anti-proliferative activity was mediated by a heparin-induced, growth arrest-specific gene named CCN5, a member of the CCN family (Delmolino et al., 1997 and 2001; Mishra-Gorur et al., 1998; Gray and Castellot, 2004). Both heparin and CCN5 localize preferentially to the abluminal surface of intimal (EC) and in the aorta to the inner media of the artery wall. This places two potent growth inhibitors in a strategic position to function as physiologic regulators of SMC function. Supporting this idea is that CCN5 rapidly disappears from the artery wall within 2 days after rat carotid artery balloon angioplasty vascular injury, and returns after 14 days, when SMC proliferation has ceased (Lake and Castellot, 2003).

Vascular remodeling is a complex process involving an increase in vascular smooth muscle cell (VSMC) proliferation as well as an increase in VSMC motility and extracellular matrix degradation to invade through the endothelial basement membrane and into the lumen (Rajagopal et al., 2003; Bennett at al., 2001; Scott et al., 2006). CCN5 activities in vitro indicate that high levels of CCN5 promote the VSMC phenotype seen in the uninjured artery wall, and loss of CCN5 is associated with an injured CCN5 phenotype. CCN5 mRNA and protein are highly expressed in the smooth muscle and endothelial cells of larger conducting vessels including coronary arteries as well as in the endocardium and myocardium (Delmolino et al., 2001; Gray et al., 2005; Gray et al., entitled “CCN5 Expression in Mammals, II. Adult rodent tissues”, Journal of Cell Communication and Signaling 2007, 1(2): 145-158, incorporated in its entirety herein by reference, and hereinafter referred to as Gray et al, 2007; Lake et al., 2003; and Malmquist et al., entitled “CCN5 Expression in Mammals, I. Embryonic and Fetal tissues of Human and Mouse”, Journal of Cell Communication and Signaling 2007, 1(2): 127-143, incorporated in its entirety herein by reference, and hereinafter referred to as Malmquist et al., 2007).

Originally discovered as a heparin-induced gene in vascular smooth muscle cells (SMC), CCN5 is shown herein to behave as a growth-arrest-specific gene in this cell type. CCN5 is highly expressed in quiescent, non-proliferating rat aortic SMC and expression levels drop rapidly as cells are stimulated to re-enter the cell cycle. SMC CCN5 expression decreases in uterine fibroids and after vascular injury, which tissue are in vivo models of SMC proliferation (Delmolino et al, 2001; Lake et al, 2003; Mason et al, 2004b). CCN5 over-expression inhibits SMC proliferation, motility, and invasiveness in vitro, and CCN5 knock-down has opposite effects (Lake et al, 2003; Lake and Castellot, 2003; Mason et al, 2004b). CCN5 is strongly up-regulated by estrogen in both SMC and epithelial cells (Fritah et al, 2006; Gray and Castellot, 2005; Inadera et al, 2000; Mason et al, 2004a).

There are several pathologic conditions in which CCN2 and CCN5 have opposite expression patterns, including asthma (unpublished observations; Burgess, 2005), uterine leiomyoma (fibroids) (De Falco et al, 2006; Mason et al, 2004b), and hepatocellular carcinoma (Cervello et al, 2004; Hirasaki et al, 2001). However, in other diseases, including arthritis (Manns et al, 2006; Tanaka et al, 2005) and viral hepatitis (Fukutomi et al, 2005; Shin et al, 2005), CCN2 and CCN5 are similarly expressed. CCN2 and CCN5 have opposite expression patterns in vascular SMC. CCN2 over-expression induces vascular SMC proliferation and increases MMP-2 expression, and CCN5 over-expression reduces proliferation and MMP-2 expression. CCN5 expression decreases but CCN2 increases in vascular SMC during the proliferative phase of balloon angioplasty injury (Ando et al, 2004; Fan et al, 2000; Fan and Karnovsky, 2002; Lake et al, 2003; Lake and Castellot, 2003). Opposite expression patterns of CCN2 and CCN5 in vascular SMC and in several proliferative disease states suggest that they might have complementary or opposite expression patterns in the developing embryo.

CCN5 is also expressed in a wide range of non-cardiovascular cells and tissue types (as shown herein; Mason et al., 2007). CCN5 expression is strongly associated with quiescent arterial SMC and negatively associated with proliferating arterial SMC both in vitro and in vivo. CCN5 is highly expressed in quiescent, non-proliferating rat aortic SMC and expression levels drop rapidly as cells are stimulated to re-enter the cell cycle. Over-expression of CCN5 using adenoviral constructs inhibits vascular SMC proliferation, and siRNA knock-down stimulates proliferation even in the presence of an anti-proliferative levels of heparin (Lake and Castellot, 2003; Lake et al., 2003). CCN5 over-expression strongly down-regulated, and knock-down increased, matrix metalloproteinases required for SMC motility and invasiveness. CCN5 knock-down reduced actin transcription and filament assembly by 50%. Knock-down or overexpression of CCN5 does not affect apoptosis in VSMCs.

A method is provided herein for treating a smooth muscle proliferation disorder in a mammalian subject, the method including expressing CCN5 in or administering CCN5 protein to smooth muscle cells, and the disorder is at least one selected from the group of: asthma, vascular injury, persistent pulmonary hypertension in the newborn (PPNH), pulmonary hypertension in adults, megaureter, pyloric stenosis, uterine fibroids, lymphangioleiomyomatosis (LAM), cervical incompetence, and cancer.

Persistant Pulmonary Hypertension of the Newborn (PPHN) is an illness that is a major cause of death among newborns. Babies that survive suffer from sever brain damage and other problems.

Pyloric Stenosis is a smooth muscle disorder in which the valve regulating food flow from the stomach to the small intestine becomes overgrown with smooth muscle and remains shut. The only known treatment involves surgery.

Cervical Incompetence is another smooth muscle disorder in which smooth muscle cell overgrowth causes the cervix to lose its “stretchability” and function. Loss of stretchability or elasticity of the cervix can lead to infertiliy and other problems.

Megaureter is a smooth muscle disorder that affects approximately 1 in 2000 infants and a similar number of adults. In this disorder, the ureter becomes thickened with smooth muscle cells, narrowing the lumen, which causes severe pain and other problems.

Lymphangioleiomyomatosis (LAM) is a progressive lung disease that affects women, usually during their childbearing years. Symptoms include collapsed lung, fluid in the lungs, shortness of breath, fatigue, cough, and chest pain. LAM is often misdiagnosed as asthma, emphysema, and pulmonary bronchitis. Scientists estimate that there may be 250,000 to 300,000 misdiagnosed or undiagnosed LAM patients.

Without being bound by any particular theory or mechanism of action, a tumor is associated with a new and continuing blood supply (angiogenesis) for growth and for metastasis. Prior research on the relationship of angiogenesis and tumor growth has focused on the capillary endothelial cell during angiogenesis, even though capillaries by themselves are not sufficient to supply a large tumor with blood. During tumor development, capillaries are remodeled into small arteries and veins, and then into larger arteries and veins. This process involves recruitment (migration) and proliferation of SMC to provide the vascular wall cells for the growing arteries and veins. Histologic and anatomic evidence presented herein indicates that CCN5 treatment of tumors arising from human breast cancer cells were observed to be significantly smaller than control tumors arising from cells not administered CCN5.

Another method is provided herein for treatment of the disorder asthma, in which disease the CCN5 is expressed in airway smooth muscle cells (ASM) by contacting ASM with an expression vector encoding CCN5, the vector selected from a virus vector and a nucleic acid vector, or contacting ASM with recombinantly produced CCN5 protein. For example, the vector is delivered by inhalation. While the term, “infecting” has been used to describe contacting airway cells with a virus vector, the term in no way implies a productive viral infection, as the vectors are genetically engineered to eliminate virulence, and to carry genetic information encoding the CCN5 or related agent. A CNN5 related agent includes any nucleotide sequence which, when contacted to a cell and enters the cell, is capable of operably expressing CNN5 protein within the cell. The nucleotide sequence includes any that are encapsulated into a virion for transduction or infection as a mode of delivery of the sequence into the cell.

In an alternative embodiment, expression of CCN5 is mediated by a small molecule or other agent that causes CCN5 to be over-expressed. Further, the small molecule or other agent is formulated for delivery to the subject, for example, by a route such as inhalation.

Also provided herein is a method for evaluating airway remodeling, the method involving: modulating the expression of CCN5 in ASM in a mammal having symptoms of chronic asthma; and assessing the morphology of ASM in comparison to a control having the symptoms and otherwise identical to the mammal and not modulated in expression of CCN5.

An additional embodiment of the invention provides a method for treating a smooth muscle disorder in a mammal including reducing the expression or activity in ASM of one or more genes that are suppressed by expression of CCN5. The one or more genes, which are representative of a set of genes considered to be CCN-5 target genes, is at least one of genes LILRA1, DEFB103A, LOC387643, LY6K and OR4X2. Accordingly, the expression of one or more genes selected from LILRA1, DEFB103A, LOC387643, LY6K and OR4X2 is reduced in a smooth muscle cell by expressing an antisense RNA, ribozyme or siRNA that targets RNA encoding one or more genes selected from the group consisting of LILRA1, DEFB103A, LOC387643, LY6K and OR4X2. The CCN5, vector encoding CCN5 of viral or non-viral origin, small molecule, and antisense RNA, ribozyme or siRNA are examples of a group of materials that are CCN5-related agents. Accordingly in certain embodiments, the expression or activity of one or more genes selected from the group consisting of LILRA1, DEFB103A, LOC387643, LY6K and OR4X2 is mediated by a small molecule or other agent that reduces the expression or activity of one or more genes selected from the group consisting of LILRA1, DEFB103A, LOC387643, LY6K and OR4X2. In various alternative embodiments, expressing an antisense RNA, ribozyme or siRNA further includes delivering a nucleic acid expression vector by inhalation. The CCN5-related agent such as the small molecule is delivered via inhalation, for example for treatment of asthma, or by some other appropriate route. Local administration in contact with or adjacent to the organ or tissue to be treated is preferred to systemic administration.

The invention provides new insights both therapeutically and by increasing our understanding of airway pathobiology. Use of animals is critical for understanding the pathogenesis of AWR and the development of effective therapeutic agents for asthma because human studies examining structural airway changes (below the epithelium) are far too invasive. Examples herein use mice chronically exposed to HDM extract to induce asthma (Johnson et al., 2004). Given that HDM is the most common indoor aeroallergen, implicated in allergic symptoms in 10% of the population, the HDM-mouse is clinically relevant. Chronic HDM induces airway eosinophilia, increased specific IgE production, airway hyper-reactivity, and multiple features of AWR. Chronic HDM treatment does not result in immunologic tolerance as seen in other animal models, such as those based on ovalbumin administration.

The invention provides new insights both therapeutically and by increasing understanding of airway, vascular and cancer pathobiology.

In one aspect, the invention provides a method for treating smooth muscle proliferation disorders in a mammal including expressing CCN5 in smooth muscle cells. In examples herein, CCN5 expression in cells is achieved by contacting the cells, for example, infecting the cells with a vector that is a virus or an expression vector encoding CCN5 (known genetically as transducing the CCN5 gene). The process of contacting a tissue or cell with CCN5 or a vector encoding CCN5 is frequently referred to herein as “pre-treating”. In some embodiments, expression of CCN5 is mediated by a small molecule or other agent that modulates CCN5, e.g., causes CCN5 to be over-expressed. In embodiments, the virus, expression vector, small molecule or other agent is delivered via inhalation. In embodiments, the mammal is a human.

Also provided herein is a method for evaluating airway remodeling including modulating the expression of CCN5 in smooth muscle cells in a mammal having symptoms of chronic asthma, vascular injury, or cancer. Modulation of CCN5 expression includes increasing expression of CCN5 in smooth muscle cells by contacting the cells with a virus or an expression vector encoding CCN5. Alternatively, expression of CCN5 is mediated by a small molecule or other agent capable of up-regulating, or causing CCN5 to be expressed. In embodiments of smooth muscle cell proliferation-based disorders, particularly an airway disorder such as chronic asthma, the disorder is treated in a mammal by inhalation. The mammal is a human, alternatively the mammal is a non-human primate or a rodent such as a rat or a mouse.

Further provided herein is a method for treating a smooth muscle cell proliferation-based disorder in a mammal by reducing the expression or activity in smooth muscle cells of one or more genes that are suppressed by expression of CCN5. As shown herein, a large number of genes are suppressed by expression of CCN5, for example, the one or more genes is selected the group consisting of LILRA1, DEFB103A, LOC387643, LY6K and OR4X2. In some embodiments, the expression of one or more genes selected from the group consisting of LILRA1, DEFB103A, LOC387643, LY6K and OR4X2 is reduced by expressing in smooth muscle cells a vector, e.g., a virus or a plasmid expression vector expressing an antisense RNA, ribozyme or siRNA that targets RNA encoding one or more genes selected from the group consisting of LILRA1, DEFB103A, LOC387643, LY6K and OR4X2. In some embodiments, the expression or activity of one or more genes selected from the group consisting of LILRA1, DEFB103A, LOC387643, LY6K and OR4X2 is alternatively mediated by a small molecule or other agent that reduces the expression or activity of one or more genes selected from the group consisting of LILRA1, DEFB103A, LOC387643, LY6K and OR4X2. In some embodiments the virus, expression vector, small molecule or other agent is delivered via inhalation.

An embodiment of the method for treating asthma includes contacting smooth muscle cells with an antisense oligonucleotide, ribozyme, or siRNA that targets one or more genes selected the group consisting of LILRA1, DEFB103A, LOC387643, LY6K and OR4X2. For example, the antisense oligonucleotide, ribozyme, or siRNA is delivered by inhalation.

Also provided herein is a method for evaluating airway remodeling in a mammal including reducing the expression or activity in ASM of one or more genes that are suppressed by expression of CCN5 and assessing the morphology of ASM. Examplary mammals include human, non-human primates, and rodents such as rat and mouse.

Exemplary of the one or more genes are LILRA1, DEFB103A, LOC387643, LY6K and OR4X2. The expression of one or more genes from the group consisting of LILRA1, DEFB103A, LOC387643, LY6K and OR4X2 is reduced by expressing in ASM a virus or expression vector expressing an antisense RNA, ribozyme or siRNA that targets RNA encoding one or more genes selected from the group consisting of LILRA1, DEFB103A, LOC387643, LY6K and OR4X2. Alternatively, the expression or activity of one or more genes is mediated by a small molecule or other agent that reduces the expression or activity of one or more genes selected from the group consisting of LILRA1, DEFB103A, LOC387643, LY6K and OR4X2. In some embodiments the virus vector, nucleic acid vector such as the expression vector, small molecule or other agent is delivered via inhalation.

In some embodiments, the method for evaluating airway remodeling includes contacting ASM with an antisense oligonucleotide, ribozyme, or siRNA that targets one or more genes selected the group consisting of LILRA1, DEFB103A, LOC387643, LY6K and OR4X2 and assessing morphology of ASM. In some embodiments, the antisense oligonucleotide, ribozyme, or siRNA is delivered by inhalation.

It is envisioned herein that CCN5 is an important downregulator of SMC proliferation, invasiveness and motility in vivo. This invention without being limited by any particular theory on or mechanism of action, is related to the discovery. herein that VSMC CCN5 levels decrease following vascular injury as the cells begin to proliferate. In addition, overexpression of CCN5 in a vascular injury model reduces the size of a vascular lesion. The expression pattern of CCN5 protein in the mouse carotid ligation vascular injury model was here analyzed, and CCN5 was found to be highly expressed throughout the media and endothelium of uninjured carotid arteries. Unexpectedly, VSMC CCN5 expression was lost in the media soon after injury and did not return to pre-injured levels up to one month following injury. Based on this surprising finding, the potential therapeutic use of CCN5 in a gene therapy model was analyzed, and CCN5 was herein found able to substantially inhibit neointimal lesion formation following a vascular event. Loss of CCN5 expression was surprisingly found herein to not to be strictly a marker of proliferating VSMC in vivo as hypothesized in previous studies but rather a marker of cells that have switched from an uninjured to an injured phenotype as indicated by loss of this marker of a growth arrest specific gene in VSMC.

### Pharmaceutical Compositions

In one aspect of the present invention, pharmaceutical compositions are provided, wherein these compositions include a CCN5 or a CCN5-related agent, and optionally include a pharmaceutically acceptable carrier. As used herein, the term, “CCN5-related agent” means a CCN5 that is a CCN-like growth factor as described in U.S. Pat. No. 5,780,263 issued Jul. 14, 1998, and includes a modulator or effector of CCN5 expression, such as a small molecule capable of modulating or upregulating, expression of CCN5 in a mammalian cell. The term further includes a modulator or effector of a gene that is regulated by CCN5, such as an antisense, a ribozyme or an siRNA that modulates CCN5 gene expression, or modulates expression of at least one of genes CCN5 target genes, exemplifed by not limited to LILRA1, DEFB103A, LOC387643, LY6K and OR4X2 as described herein.

In certain embodiments, these compositions optionally further include one or more additional therapeutic agents. In certain embodiments, the additional therapeutic agent or agents are selected from the group consisting of growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 shows various carriers used in formulating pharmaceutical compositions and known techniques for the preparation thereof. Some examples of materials which can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Therapeutically Effective Dose

In yet another aspect, according to the methods of treatment of the present invention, the treatment of a smooth muscle proliferation disorder is promoted by contacting the smooth muscle cell or tissue with a pharmaceutical composition, as described herein. Thus, the invention provides methods for the treatment of smooth muscle cell proliferation disorder, including administering a therapeutically effective amount of a pharmaceutical composition including active agents that include CCN5 and/or a CCN5-related agent to a subject in need thereof, in such amounts and for such time as is necessary to achieve the desired result. It will be appreciated that this encompasses administering an inventive pharmaceutical as a therapeutic measure to promote modulate expression of CCN5 and/or genes regulated by CCN5, to promote an appropriate amount of smooth muscle or as a prophylactic measure to minimize complications associated with present modes of treatment of a smooth muscle proliferation-based disorder (such as restenosis following balloon angioplasty for arterial plaque). In certain embodiments of the present invention a “therapeutically effective amount” of the pharmaceutical composition is that amount effective for promoting an appropriate amount of smooth muscle proliferation such that overgrowth does not occur or recur.

The compositions, according to the method of the present invention, may be administered using any amount and any route of administration effective for treating the smooth muscle proliferation-based disorder. Thus, the expression “amount effective for treating a smooth muscle proliferation-based disorder”, as used herein, refers to a sufficient amount of composition to beneficially decrease an unwanted excessive growth of a smooth muscle tissue, following a surgical procedure, or a vascular injury, or to prevent or decrease frequency and/or intensity of episodes of a chronic condition such as asthma.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., smooth muscle location and extent; age, weight and gender of the patient; diet, time and frequency of administration; drug combinations; reaction sensitivities; and tolerance/response to therapy. Long acting pharmaceutical compositions might be administered every 3 to 4 days, every week, or once every two weeks depending on half-life and clearance rate of the particular composition.

In embodiments, the active agents of the invention are formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, usually mice, rabbits, dogs, or pigs according to methods provided herein for example, for evaluating airway remodeling. The animal model is also used to achieve a desirable concentration range and route of administration. Such information can then be used to determine useful doses and routes for administration in humans. A therapeutically effective dose refers to that amount of active agent which ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies are used in formulating a range of dosage for human use.

### Administration of Pharmaceutical Compositions

The pharmaceutical compositions of CCN5 or CCN5-related agents, formulated in a desired dosage with an appropriate pharmaceutically acceptable carrier, are administered to humans and other mammals topically (as by powders, ointments, or drops), orally (as by tablets, liquids, or inhalation by aerosol), rectally, parenterally, intracisternally, intravaginally, intraperitoneally, bucally, ocularly, or nasally (as by inhalation via insufflation), depending on the severity and location of the smooth muscle proliferation disorder being treated.

Aerosol preparations suitable for inhalation may include solutions and solids in powder form, which may be in combination with a pharmaceutically acceptable carrier, such as an inert compressed gas, e.g. nitrogen. Aerosol formulations are placed into pressurized acceptable propellants, such as dichlorodifluoromethane, propane, nitrogen, and the like.

For example, administration by inhalation of the pharmaceutical compositions herein is conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In a pressurized aerosol, the dosage unit is conveniently determined by providing a valve to deliver a metered amount. Capsules and cartridges of gelatin for use in an inhaler or insufflator may be formulated containing a powder mix of CCN5 and a suitable powder base such as lactose or starch.

Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, smooth muscle cell proliferation disorders may be treated with aqueous drops, a mist, an emulsion, or a cream. Administration may be therapeutic or it may be prophylactic. Prophylactic formulations may be present or applied to the site of potential smooth muscle proliferation such as organs, tissue or vasculature having smooth muscle, such as bronchia, lung, cardiac tissue, and tumors such as brain, lung, head and neck and esophageal tumors

The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the agents of this invention, excipients such as talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleicacid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain pacifying agents and can also be of a composition that they release the active agent(s) only, or in a certain part of the intestinal tract, optionally, in a delayed manner. Examples of embedding compositions which can be used include polymeric substances and waxes.

### Uses of Parmaceutical Compositions

As discussed above and described in greater detail in the Examples herein, CCN5 and CCN-5-related agents are useful to treat or even prevent smooth muscle proliferation-based disorders, such as vascular injury, asthma, and in cancer such as cancer metastasis. The CCN5 and CCN-related agents are believed to be clinically useful to treat typical examples of vascular injury, including but are not limited to tensile strain, shear strain, vessel rupture, intimal rupture, penetrating trauma, blunt trauma, transection, contusion, laceration, arteriovenous (AV) fistula formation, vessel spasm, external compression, mural contusion, thrombosis, and aneurysm formation. Further, the vascular injury involving stenosis or restenosis is associated with at least one of wire injury, ligation injury, arteriovenous shunt, coronary artery bupass graft, endarterectomy, hypertension and balloon angioplasty.

The following examples are intended to further illustrate certain embodiments of the invention, and are not to be construed to limit the scope of the invention. Additional examples and embodiments can be found in references published in 2007 in the Journal of Cell Communication and Signaling, entitled, “CCN5 Expression in Mammals, I. Embryonic and Fetal tissues of Human and Mouse”, by J. A. Malmquist, M. R. Gray, B. E., Oliveira, M. Loch, and J. J. Castellot, 1(2): 127-143, and “CCN5 Expression in Mammals, II. Adult rodent tissues”, by M. R. Gray, J. A Malmquist, M. Sullivan, M. Blea, and J. J. Castellot, 1(2): 145-148, and which are both hereby incorporated by reference herein in their entireties.

### EXAMPLES

The following materials and methods were used throughout the examples herein.

Frozen Tissue Preparation and Staining

Arteries were carefully removed by dissection and perfused, following perfusion with ice-cold phosphate buffered saline without calcium or magnesium (PBS) (Hyclone; Logan, Utah) and then 4% paraformaldehyde (Fisher Scientific) in PBS with experimental manipulations as described. Arteries were rinsed in cold PBS buffer and placed in 4% paraformaldehyde for 24 hours at 4° C. Fixed samples were transferred to 30% sucrose in PBS for 24 hours and then frozen in embedding medium (Tissue-Tek OCT; Sakura Finetek USA Inc; Torrance, Calif.). Blocks of frozen tissue were stored at −70° C. until sectioning (12 μM) using a Leica CM3050S cryostat (Leica Microsystems, Deerfield, Ill.) and glass slides (Superfrost/Plus; Fisher Scientific). All slides were stored at −20° C., and were removed for staining with Verhoeff's Elastic Stain Kit (Newcomer Supply; Middleton, Wis.) according to the manufacturer's instructions or immunohistochemical analysis for CCN5 using the Vectastain Elite ABC kit (PK 6101; Vector Laboratories, Burlingame, Calif.), or for BrdU using the Vector M.O.M. Peroxidase Kit (PK2200; Vector Laboratories) and developed with the 3,3′-diaminobenzidine (DAB) substrate kit (SK4100; Vector Laboratories). CCN5 protein was detected using a well-characterized affinity-purified rabbit polyclonal antibody to a mouse polypeptide fragment from amino acids 103-117 of the von Willebrand Factor-C (VWC) domain of CCN5 (Gray et al., 2005; 24-27). The specificity of the anti-CCN5 antibody was verified by Western blot analysis of cultured rat aortic SMC lysates. A prominent 28 kDa band is produced in growth-arrested rat VSMC or in rat VSMC transfected with AdCCN5 (Lake and Catellot, 2003; Gray et al., 2004; and Lake et al., 2003). Briefly, endogenous peroxidase activity was quenched by treatment with 0.3% hydrogen peroxide (H₂O₂; Fisher Scientific) in methanol (Fisher Scientific) for 30 minutes. Slides were blocked at room temperature for 20 minutes then incubated with anti-CCN5 (1:100 in PBS) overnight at 4° C. PBS in place of persistent antibody was used as a negative control. Negative controls observed to completely lack brown staining. After incubation with the secondary antibody and the ABC reagent (30 minutes each at room temperature), slides were developed in DAB without Nickel Solution for 6 minutes and counterstained for 10 seconds with Harris modified hematoxylin with acetic acid (Fisher Scientific).

For BrdU analysis, immunohistochemistry (IHC) sections were boiled in 10 mM citric acid pH 6.0 (Fisher Scientific) for 20 minutes for antigen retrieval, and were incubated with primary anti-BrdU antibody (BD Biosciences; San Jose, Calif.) at a concentration of 1:100 overnight at 4° C. Slides were developed with the DAB substrate kit prepared with Nickel Solution for 20 minutes and counterstained for 10 seconds with Harris modified hematoxylin with acetic acid. For GFP analysis carotid arteries from animals sacrificed 2 days following AdGFP treatment were fixed, frozen, and sectioned as described above. Endogenous enhanced GFP fluorescence due to expression of the AdGFP virus was directly detected as previously described (Kusser et al., 2003; Shariatmadari et al., 2001).6

Histochemically analyzed slides were dehydrated and embedded in permanent mounting medium (#13510; DPX Mountant; Electron Microscopy Sciences; Hatfield, Pa.) and photographed using a microscope (Zeiss Axioscope) and a digital camera system (SPOT; Diagnostic Instruments). Directly compared images are from slides processed in a single experiment.

The medial area was calculated by subtracting the area defined by the internal elastic lamina (IEL) from the area defined by the external elastic lamina (EEL), and the neointimal area was calculated by subtracting the area defined by the lumen from the area defined by the IEL using image analysis software (ImagePro 6.0, MediaCybernetics, Bethesda, Md.). For morphometric studies three carotid artery sections per animal were analyzed from the midpoint between the location of the second (or only) ligation and the placement of the clamp (3 sections taken 1.0 mm, 1.25 mm, and 1.5 mm proximal from the ligature). Distance from the ligature was determined by counting the number of sections taken. Results herein and of others (Kumar et al., 1997; Pires et al., 2006; Xu, 2004) with this model show that the degree of lesion formation changes somewhat as one moves further from the superior ligation site, thus for comparative or quantitative morphometric, histologic, molecular, and biochemical measurements, tissue was taken from the same place in the artery relative to the superior ligature.

Data are presented as the mean±SEM. Significance of difference was assessed using an ANOVA analysis. Differences were considered significant at a value of P<0.05.

### Animal Embryos

Animal protocols were reviewed and approved by The Institutional Animal Care and Use Committee at Tufts University (Boston, Mass.). C57BL/6J mice (Jackson Laboratories, Bar Harbor, Me. were obtained from Charles River Laboratories (Wilmington, Mass.). Food and water were available ad libitum. Mouse gestational ages were confirmed by comparison to a mouse development atlas (Kauffman, 1992). Mice of gestational age 9-13 days post coitum are referred to as embryos (E9-E13). Older developing mice are referred to by gestational day (GD14-GD16).

### Immunohistochemistry of Animal Embryos

Paraffin-embedded 7 μm sagittal sections of C57BL embryos and fetuses (FD Neurotechnologies, Baltimore, Md.) and paraffin-embedded 5 μm sections from tissue cores in a human normal fetus tissue array (BE01014 and BE01015; US Biomax, Rockville, Md.) were cleared with xylenes. Endogenous peroxidase activity was quenched by treatment with two changes of 0.6% hydrogen peroxide (H₂O₂) in ethanol for 5 minutes. The slides were then rehydrated and treated with the Avidin/Biotin Blocking Kit (Vector Laboratories) in blocking serum (4% bovine serum albumin and 2% goat serum in phosphate-buffered saline) and then incubated in primary antibody in blocking serum overnight at 4° C. CCN5 protein was detected using a well-characterized peptide affinity-purified rabbit polyclonal antibody to a polypeptide fragment from the von Willebrand Factor-C (VWC) domain of CCN5 (Gray and Castellot, 2005; Lake et al, 2003; Mason et al, 2004a). CCN2 protein was detected using a peptide affinitypurified rabbit polyclonal antibody to polypeptide fragment amino acids 223-348 from the thrombospondin-1 (TSP) and carboxy-terminal (CT) domains of mouse CCN2 (ab6992; Abcam, Cambridge, Mass.). Purified rabbit immunoglobulin IgG was used as a negative control (Biomeda, Foster City, Calif.). Data obtained from the negative controls in these examples were observed to completely lack any brown staining. The anti-CCN2 antibody has been used previously in numerous immunohistochemical studies (Candido et al, 2003; Dean et al, 2005; Finckenberg et al, 2003; Razzaque et al, 2003). Slides were developed using the Vectastain Elite ABC kit (Vector Laboratories, Burlingame, Calif.) and the 3,3′-diaminobenzidine (DAB) substrate kit (Vector Laboratories) and counterstained with Harris modified hematoxylin with acetic acid (Fisher). Slides were dehydrated and embedded in permanent mounting medium (#13510; DPX Mountant; Electron Microscopy Sciences; Hatfield, Pa.) and photographed using a microscope (Zeiss Axioscope) and a digital camera system (SPOT; Diagnostic Instruments). Antibody concentrations and substrate exposure times were carefully titrated to minimize artifacts and ensure that the staining intensities produced by both antibodies were similar. All directly compared images are from slides processed in a single experiment with a matched negative control (purified rabbit immunoglobulin IgG).

### Reverse Transcriptase PCR (RT-PCR)

Two GD14.5 mice were sacrificed with carbon dioxide (CO2) overdose. Fetuses were dissected and immediately placed in RNAlater (QIAGEN, Valencia, Calif.) and stored at −20° C. The dissected tissues were later removed from storage and 10 mg of each tissue was homogenized using a rotor/stator homogenizer (Fisher Scientific, Pittsburgh, Pa.). RNA isolation was performed using the RNeasy Mini kit (QIAGEN). DNA was removed using RQ1 RNase-Free DNase (Promega, Madison, Wis.), and reverse transcription was performed using the RETROscript kit (Ambion, Austin, Tex.). PCR assays were performed according to the manufacturer's protocol. Control reactions with no reverse transcriptase were used to check for genomic DNA contamination in each sample. PCR was performed using the HotStarTaq Master Mix kit (QIAGEN) with 95° C. 15 min polymerase activation step followed by 35 cycles of 94° C. 30 sec/50 C 30 sec/72° C. 1 min and final 72° C. 10 min extension step and products were examined on a 1.5% agarose gel containing ethidium bromide. Primers were purchased from Integrated DNA Technologies (IDT, Coralville, Iowa). The sense CCN5 (GenBank Accession no. GI 4028578) primer consisted of the DNA sequence 5′-ATACAGGTGCCAGGAAGGTG-3′ (position 707-726; SEQ ID NO: 1), and the sequence of the anti-sense CCN5 primer was 5′-GTTGGATACTCGGGTGGCTA-3′ (position 913-932; SEQ ID NO: 2). After PCR, these primers produced a 225 bp (base pairs) DNA fragment that included the exon 4-5 (VWC-TSP) boundary in order to prevent amplification of genomic DNA sequence. The amplified DNA fragment was purifed by electrophoresis and the QIAquick Gel Extraction kit (QIAGEN) and sequenced by the Tufts University Core Facility (Boston, Mass.) to verify its identity. A plasmid containing mouse CCN5 cDNA was used as a positive control for PCR. Both water and mRNA not treated with reverse transcriptase enzyme were used as negative controls. PCR was also performed with the above conditions for the reference gene TATA box binding protein (Thp) (GenBank Accession no. GI 2052376) with the following primers sense 540 -CCTCTCAGAAGCATCACTA-3′ (SEQ ID NO: 3) and antisense 5′-GCCAAGCCCTGAGCATAA-3′ (SEQ ID NO: 4). PCR produced a 166 bp DNA fragment that included an exon-exon boundary (Willems et al, 2006).

### Example 1

Time course of HDM Exposure in an Animal Model and Disease Progression

Normal BALB/c mice that are 6-8 weeks old were exposed to 25 μg of commercially-prepared purified HDM extract or saline (control mice) intranasally for 5 days per week for up to 10 weeks (Johnson et al., 2004). During this period, the HDM-exposed animals displayed the entire disease progression, from mild asthma to severe asthma with AWR, with airway hyper-responsiveness beginning at the 5th week. To evaluate asthma progression, several tests were performed, including eosinophil counts in bronchoalveolar lavage fluid and serum cytokine measurements as previously described (Evans et al., 2003). Cell counts and differential staining were performed, and cytokine levels of IL-4, IL-13, IL-5 and TNF-α were performed at 2-week intervals after exposure to HDM. Total IgE and HDM-specific IgE levels in the serum were measured using antigen-capture ELISA. Respiratory function was assessed by measurements of airway resistance and lung compliance using the methacholine dose-response method (Glaab et al., 2005; Evans et al., 2003). Airway hyper-responsiveness to methacholine challenge is a well-characterized measure of respiratory function, especially the assessment of airway resistance.

Airway morphometric measurements were used to assess the thickness of bronchial walls using standard methods (Kuhn et al., 2000). Bronchial lumen diameter, epithelial thickness, and the bronchial wall thickness were measured. The ASM in the trachea and bronchi of the lung demonstrated the appropriate morphological and functional changes during the progression to AWR associated with chronic asthma.

CCN5 expression in animals made asthmatic by the above procedure, in comparison to control untreated animals, was analyzed as shown in Example 2.

### Example 2

Analysis of Diseased and Normal Tissues for CCN5 Expression

Lung and trachea samples were obtained from test and control animals at baseline (before HDM exposure), after 1 week, 3 weeks, 5 weeks, 7 weeks, and 10 weeks. Animals were euthanized by carbon dioxide overdose followed by cervical dislocation, followed by immediate tissue sampling. Immunohistochemistry (IHC) to analyze gene expression was performed on tissue samples as described previously (Mason et al., 2004a). Two portions of tissue from each site were frozen at −80° C. for preparation of mRNA and tissue protein lysate preparation. Fresh samples of trachea were used to prepare cultured ASM as described below. Histological analysis of HDM ASM was carried out using hematoxylin and eosin, trichrome, and Sirius red using standard protocols and examined for gross histological changes in the trachea and lung during the course of asthma development in the HDM stimulated mouse, and comparisons were made between baseline (before HDM exposure), and after 1 week, 3 weeks, 5 weeks, and 7 weeks.

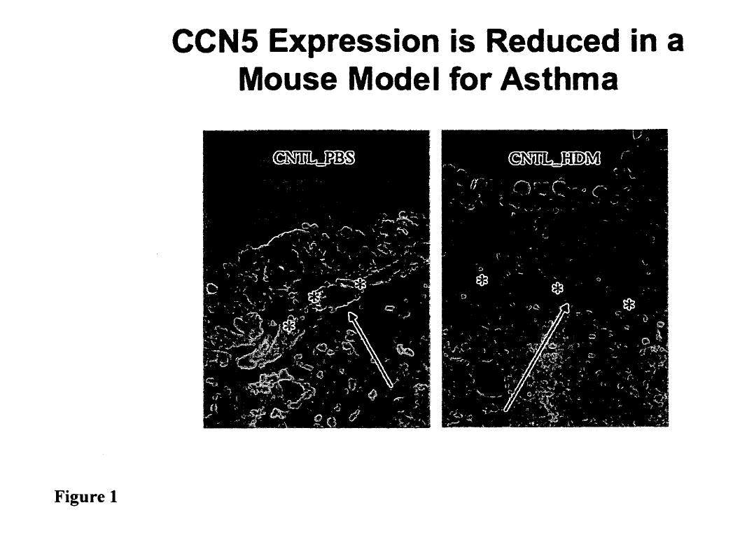

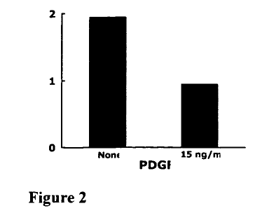

CCN5 analysis by IHC of normal and HDM ASM was performed using each of frozen and paraffin sections of lung and trachea and antibodies for CCN5 that well-characterized from previous VSM and UtSM examples. CCN5 expression patterns were examined in normal lung and trachea and compared to that of tissues obtained at baseline (before HDM exposure), and after 1 week, 3 weeks, 5 weeks, and 7 weeks. Slides were stained for CCN5 expression using the DAB-peroxidase as previously described (Mason et al., 2004a). FIG. 1 shows that CCN5 expression was found to be greatly reduced in smooth muscle cells of the airway after 7 weeks of exposure to HDM, in contrast to smooth muscle cells of control mice not exposed to this asthma-inducing material. The lack of CCN5 expression in the smooth muscle cells could thus be causally associated with the disease state.

Further, mitogenic stimulation of normal human ASM in culture caused a rapid reduction in CCN5 mRNA (FIG. 2, right lane), by a factor of about 50%. These data show that amount of CCN5 is modulated by each of the cell cycle and also by a disease state.

### Example 3

Preparation and Delivery of CCN5 Protein to the HDM Mouse

A quantity of a vector for CCN5 expression, AdCCN5, was prepared using the Adenovirus Core Facility at Tufts. Other sources of CCN5 used herein are plasmid expression vectors, and recombinantly produced CCN5 protein. The plasmid expression vectors (Mason et al., 2003; Lake and Castellot, 2003) were used to prepare adenovirus. Human, rat, and mouse CCN5 expression plasmids are commercially available, and mouse CCN5 expression plasmids were initially selected to be used in examples herein in which the recipient subjects were mice or mouse cells. Further, because the sequences of the CCN5 gene and protein are highly conserved among vertebrates, it is likely that human CCN5 would produce the same response as mouse CCN5 in a mouse model system. However, mouse recombinant CCN5 was produced for administration in the event that the human protein might produce an immunologic reaction in the mouse.

Initially each of an adenovirus vector AdCCN5, a CCN5 expression plasmid, and purified recombinant CCN5 protein were administered to subjects or cells intranasally, similar to method of administration of HDM extract. The HDM extract was not combined with the CCN5 preparations to avoid a potential direct cross-reactions between CCN5 and components of the HDM extract. The efficacy of CCN5 delivery to the airway was assessed. Mice administered CCN5 according to each of the three vectors above (adenovirus, plasmid, or recombinant protein), were sacrificed at time points up to three days later. Trachea and lung tissue samples were removed and CCN5 was analyzed by CCN5 IHC. The CCN5 dose was adjusted as needed so that the amounts of CCN5 in airway samples were similar to that of undisturbed quiescent airway, as determined by Western blot analysis. CCN5 administration was further tested using a method based on delivery to the animals by aerosols containing high concentrations of CCN5. New aerosol-based lung delivery methods that have been demonstrated to be effective were also used (Baelder et al., 2005; Gautam et al., 2001). A control adenovirus vector (AdGFP) carrying a gene for green fluorescent protein and no CCN5 gene, was administered to HDM mice, to determine whether adenoviral infection of ASM in vivo produced any adverse effects.

Dosage of CCN5 administration was initially based on dose-response data from cultured ASM, and from estimates of normal CCN5 levels in quiescent ASM in vivo using Western blot analysis. CCN5 was administered for 5 days a week according to the schedule for prior administration of HDM. Immunologic response to any of adenovirus infection, plasmid DNA transfection, or human protein was closely monitored in the mice, and, as needed, both dosage and regimens were adjusted to eliminate such a response.

### Example 4

Analysis of the Responses to Exogenously Administered CCN5

The airway responses to exogenously administered CCN5 were analyzed in mice exposed to HDM extract using the methods described above. Animals were monitored for 10 weeks from the beginning of CCN5 administration. Respiratory function tests were based on measurements of airway dose-response curves to intravenous methacholine as described above. These tests were performed every two weeks following the first administration of CCN5. Airway morphometric measurements to determine bronchial wall thickness were performed as described above. At two week intervals, trachea and lung samples were isolated and analyzed histologically and by IHC for the expression of CCN5, CCN2, smooth muscle α-actin, and matrix metalloproteinases and inhibitors: MMP-9, MMP-8, MMP-2, MMP-12, MT1-MMP, and TIMPs 1 and 2.

Without being bound by any particular theory or mechanism of action, it was envisioned that at least one of these preparations and delivery methods would produce a similar result to that seen when CCN5 is over-expressed in a vascular injury model provided in the methods herein, namely, that CCN5 strongly suppresses airway remodeling associated with disease, and thereby improves pulmonary function in the mouse model system. These examples herein provide data linking structural changes in the airway to pulmonary function changes. Furthermore, the degree of structural changes observed in the presence or absence of CCN5 matched the degree of pulmonary function changes measured.

### Example 5

CCN5 Regulation of ASMC Function in Cell Culture

These examples show that CCN5 regulates critical functions of ASM, using well-characterized cell culture systems in which variables are controlled with greater precision than in animal models.

Normal human airway smooth muscle (ASM) cell cultures were used to compare to mouse ASM cell cultures generated in these studies in order to determine whether these species show differences in CCN5 expression. The human cell line has been described (Krymskaya et al., 2001) and was obtained from a portion of trachealis muscle from a healthy lung transplant donor. These cells retain native contractile protein expression as well as the signaling pathways that are characteristic of functional ASM. Tracheal and bronchial samples were isolated for preparation of cultured murine ASM for use in the examples described below, for analysis of ASM at all stages of asthma. Tracheal and bronchial samples were excised and transferred immediately to ice-cold buffer. ASM were isolated and cultured using a standard method (Deshpande et al., 2004). The smooth muscle phenotype was confirmed by IHC staining using antibodies to smooth muscle-specific α-actin and smooth muscle myosin heavy chain. The vector AdCCN5 was used for the examples below that describe observations of constitutive over-expression of CCN5 in ASM.

Adenovirus vectors were constructed as follows: a control vector was constructed capable of expressing green fluorescent protein (GFP), and a working functional vector was constructed capable of expressing each of GFP and a CCN5 cDNA sequence that additionally includes a nine amino acid HA epitope tag located at the C-terminus. These two genes in the construct AdCCN5 vector are under control of different CMV promoters. To administer CCN5, cells or tissues such as ASM or VSM was contacted to each of the functional CCN5 and control vectors, and success of infection was monitored as previously described (Lake et al., 2003). Further, administration of each of the expression plasmid and recombinant CCN5 protein described above was included in these studies.

CCN5 knockdown by siRNA transfection using an siRNA was previously reported to suppress CCN5 protein levels in VSM and UtSM by more then 80% enabling a test of the role of CCN5 by monitoring results from loss-of-function examples (Lake and Castellot, 2003). Proliferation rates were assessed by cell counting using a Coulter particle counter. Proliferation was measured in unmodified ASMC, in CCN5 over-expressing cells (AdCCN5 infected), and in CCN5 knockdown cells (siRNA-CCN5 transfected). Motility in these same cells was assessed using the scratch-wound, modified Boyden chamber, and Matrigel invasion assays previously described for VSM (Lake and Castellot, 2003).

The data obtained from these examples show that: cultured ASM cell phenotype was similar to that of vascular smooth muscle (VSM) and uterine smooth muscle (UtSMC); CCN5 levels were found to be highest during ASM growth arrest, and lowest during cell proliferation; over-expression of CCN5 suppressed proliferation and motility of each ASM tested, including the animal asthma model, HDM-exposed mice; and CCN5 knockdown increased rates of proliferation and motility in all ASMC tested, including those from the HDM-exposed mice.

### Example 6

Role of CCN5 in Regulation of Proteins

Cultured ASMC from the above examples were tested for CCN5 protein expression using Western blots (by methods described in Mason et al., 2004a). ASM was tested for CCN5 protein and mRNA levels in each of human ASMC and mouse ASMC obtained from each of the different HDM time points discussed above; including the unmodified, CCN5 over-expressing, and CCN5 knockdown cells and control unmodified cells. CCN5 regulation of several potential downstream target proteins was analyzed by Western blot analysis of the same lysates prepared from the panel of cultured ASM for the above CCN5 Western blot examples. mRNA levels also were analyzed by Q-PCR.

Major intracellular proteins were assayed as follows: smooth muscle α-actin, myosin light chain 2 (and its level of phosphorylation), calponin and caldesmon. Because MMPs have been demonstrated to play a role in SM motility, the methods of zymography and Q-PCR were used to measure the activity and mRNA levels of each of MMP-2, MMP-8, MMP-9, MMP-12, MT-1, and TIMPs-1 and -2.

Western blot and Q-PCR data for CCN5 matched that observed in previous VSMC examples, i.e., levels of each of CCN5 protein and mRNA were found to be highest when ASMC were in a quiescent state, and lowest during proliferation. It was envisioned that some but not all MMPs tested would be significantly decreased in ASM from the above examples that have normal or increased levels of CCN5 expression. Myosin light chain 2 phosphorylation levels and those of calponin and caldesmon were found to be abnormal in ASM from the above examples that have decreased CCN5 expression.

### Example 7

CCN5 Target Genes in Airway Smooth Muscle

In order to identify the extracellular binding partners and intracellular signaling pathway components that are utilized during CCN5 regulation of smooth muscle cell proliferation and motility, mRNA expression profiles of human airway smooth muscle cells (hASM) infected with adenovirus expressing CCN5 were compared to that of cells contacted with GFP under control of separate CMV promoters (AdCCN5 ) to that of hASM cells contacted with an adenovirus expressing GFP alone (AdGFP). RNA prepared from cultured hASM cells contacted with either AdCCN5 or AdGFP was used as a template to prepare cDNA using reverse transcriptase.

The cDNA prepared from AdCCN5-contacted cells was labeled with Cy3 fluorescent dye and cDNA prepared from AdGFP-contacted cells was labeled with Cy5 fluorescent dye. The two fluorescently-labeled cDNA preparations were combined together in equal amounts and the mixture was applied to glass slides that had previously been spotted with microarrays of oligonucleotides from the HEEBO (Human Exonic Evidence Based Oligonucleotide) set, made from 44,308 different human genes. After several washing steps to remove non-specifically bound cDNAs, the slides were digitally scanned to reveal the intensity of dye binding to each oligonucleotide spot. Oligonucleotide spots that displayed Cy3 signal indicated genes that were expressed predominantly in the AdCCN5-contacted cells. Those that displayed Cy5 signal indicated genes that were expressed predominantly in the AdGFP-contacted cells. Those that displayed both Cy3 and Cy5 signals indicated genes that were expressed at a similar rate in both test and control hASM samples. The instrument that recorded the microarray scans also quantitatively compared the Cy5 and Cy3 fluorescent dye signals and calculated the magnitudes of mRNA expression differences between the two cDNA preparations for each gene represented on the microarray slide. To overcome array-to-array variability, three identical microarray slides were hybridized with the same cDNA preparations, producing three sets of expression comparisons.

The data showed that over 500 different genes demonstrated alterations in expression levels resulting from contact with the vector causing CCN5 over-expression. The vast majority of changes were decreases in the level of gene expression, showing that CCN5 acts primarily as a suppressor or down-regulating modulator of gene expression in hASM. Five genes were observed to have the largest decreases in CCN5 modulated expression: 4.5-fold reduction in LILRA1, a monocyte/macrophage immune system modulator membrane-bound receptor; 3.7-fold reduction in DEFB103A, antimicrobial peptide induced in bronchial epithelial cells that is associated with the inflammatory response in asthma; 3.7-fold reduction in LOC387643, a protein related to beta amyloid proteins, known to modulate lung fibrocytes and promotion of fibrosis; 3-fold reduction in LY6K, the lymphocyte antigen 6 at the K locus gene, a membrane protein of T cells; and OR4X2, an olfactory membrane receptor G-protein that activates adenylate cyclase that is not known to be expressed outside the olfactory epithelium.

Other genes observed herein as having expression modified by CCN5 included modulators of the extracellular matrix (e.g. collagens XII and XIV, both linker collagens), thrombospondin-3, ICAM1, numerous regulators of smooth muscle synthesis, many proteins that are components of cell signaling pathways, and modulators of the asthma-associated inflammatory response.

The data obtained in this example show that CCN5 expression causes a wide-spectrum of alterations of gene expression, many of which that reduce or inhibit the expression of numerous mechanisms of inflammation and airway remodeling in smooth muscle cell proliferation-based disorder such as vascular injury and chronic asthma.

### Example 8

CCN5 in the HDM Mouse Model

To test whether CCN5 has a role in regulation of ASM function in vivo and is a potential therapeutic agent in the treatment of chronic asthma, exogenous CCN5 was administered to HDM-exposed mice and prevention and arrest of ASM hyper-proliferation and airway remodeling (AWR) was analyzed. A recombinant adenovirus that expresses high levels of human CCN5 (AdCCN5) was administered to airways of mice during HDM extract exposure. Because the sequences of the CCN5 gene and protein are highly conserved among vertebrates, it was considered likely that the human CCN5 produces the same response as that of mouse CCN5 in the HDM-exposed mice. The HDM extract and the AdCCN5 preparations were not administered as a combined dose, in order to avoid any potential cross-reactivity between CCN5 and components of the HDM extract.

CCN5 was administered to the mice from three weeks after the beginning of HDM exposure (after the onset of the first phase of asthma) to five weeks after HDM (after the onset of ASM hyper-proliferation). The following groups of animals were analyzed: (1) four animals exposed to intranasal HDM for an entire 5 week test period and to intranasal AdCCN5 for weeks 3-5; (2) four animals exposed to HDM for all 5 weeks and recombinant adenovirus that expresses a negative control protein (AdGFP) for weeks 3-5; (3) two control animals exposed to HDM for all 5 weeks only; and (4) two control animals exposed to AdGFP only for weeks 3-5. Animals in groups 1-3 above were administered HDM for 5 days each week, and recombinant adenovirus doses of 1×10⁹ pfu (plaque forming units) were administered twice during weeks 3-5 (groups 1, 2, and 4). The adenovirus dosage was determined from data obtained in previous examples that utilized intranasal administration of recombinant adenoviruses to mice. The adenovirus dose in this example was specifically chosen as a low amount in order to avoid potential inflammatory effects in the airway potentially caused by the presence of adenovirus alone.

At the end of the five-week HDM exposure period, animals were tested for respiratory function to estimate airway dose-response after intravenous methacholine (MCh) administration. After the tests of airway function, animals were sacrificed by thoracotomy to examine airway histologies. Trachea and lung samples were isolated and fixed, and paraffin sections are analyzed by conventional histology and by immunohistochemistry for the expression of CCN5. Blinded results of airway function testing and airway morphology were analyzed. None of the animals in the pilot study displayed symptoms of illnesses or behavioral changes during the tests, suggesting that administration and expression of recombinant CCN5-expressing adenovirus does not produce a significant ill effect in the animals.

Recombinant mouse CCN5 protein was also administered to the HDM mouse, initially in dosages based on dose-response data from cultured ASMC and estimates of normal CCN5 levels in quiescent murine ASM. The CCN5 dosages were adjusted as needed so that the CCN5 levels in airway samples were similar to that of undisturbed quiescent airway. CCN5 was also administered using a method of exposing to aerosols formulated to contain high concentrations of CCN5.

### Example 9

Mouse Model of Carotid Ligation

Animal protocols were reviewed and approved by The Institutional Animal Care and Use Committee at Tufts University (Boston, Mass.). C57BL/6J mice were obtained from Charles River Laboratories (Wilmington, Mass.). Food and water were available ad libitum.

Acute occlusion of the left common carotid artery was performed as described (Kumar et al., 1997). Briefly, the left common carotid artery of 20 to 25 g male C57BL/6J mice anesthetized with 2.5% isofluorane (Abbot Laboratories; North Chicago, Ill.) was exposed through a midline incision, isolated, and ligated at the point of the carotid bifurcation. For the time course studies mice were sacrificed 2, 5, 8, 11, 14, 21, 28, 56, or 84 days following carotid ligation (4-6 mice at each time point). Intravascular adenovirus treatment was achieved by placing a clamp (#00396-01; Fine Science Tools; Foster City, Calif.) 3 mm proximal to the ligation and injecting 10 μl of 5×10⁹ pfu/ml adenovirus or as a control, sterile saline (IVX Animal Health, Inc.; St. Joseph, Mo.) into the carotid lumen with a 33 gauge needle and 50 μl syringe (Hamilton Company; Reno, Nev.) immediately distal to the ligation needle by piercing the wall of the artery and injecting the treatment. Following incubation for 25 minutes the puncture site was closed with a second ligation and blood flow restored to the point of the second ligation by release of the clamp. Mice were sacrificed 14 days after injury and carotid arteries analyzed as described below. The right uninjured carotid artery was used as control tissue. Four (4) mice received saline treatment, 7 mice received AdGFP (two were sacrificed 2 days following injury for infection analysis as described below) and 5 mice received AdCCN5. One mouse treated with AdCCN5 was not analyzed due to technical surgical difficulties that resulted in excessive vascular injury. For sham operations (4 mice) arteries were surgically isolated but not ligated, and animals were sacrificed 14 days following the procedure. Analgesia was provided with a single dose of subcutaneous 75 μg/kg buprenorphine HCl (Reckitt Benckiser Pharmaceuticals; Richmond, Va.) in 1 ml sterile saline (to prevent dehydration); animals showed no signs of pain or distress when evaluated 12 hours after surgery. Animals were sacrificed by carbon dioxide (CO₂) overdose followed immediately by thoracotomy. To label cycling cells in animals analyzed for time course studies animals were injected intraperitoneally with sterile 25 mg/kg 5-Bromo-2′-deoxyuridine (BrdU) (Sigma-Aldrich; St. Louis, Mo.) dissolved in 1.05 M sodium bicarbonate (NaHCO₃) (Fisher Scientific; Fair Lawn, N.J.) 12 and 1 hour(s) prior to sacrifice. Cycling cells of adenovirus- and saline-treated animals were labeled by providing 0.8 mg/ml BrdU in their drinking water following surgery as previously described (Gawronska-Kozak et al., 2004).

Previous reports show that CCN5 is reduced during the proliferative phase of rat carotid artery balloon angioplasty (Lake et al., 2003). Examples herein examine the temporal and spatial expression pattern of CCN5 in vascular injury using the mouse carotid ligation vascular injury model. The mouse carotid ligation model results in a reproducible neointima formation in mice (Kumar et al., 1997; Xu, 2004). Therefore, these data indicate that CCN5 has a role in preventing restenosis.

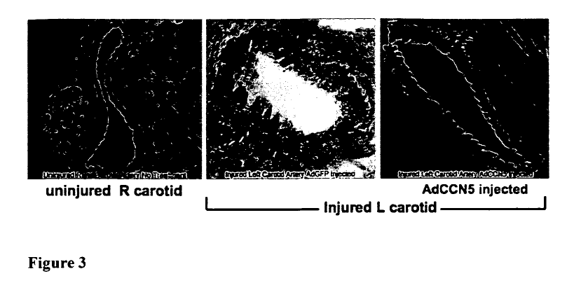

Results shown in FIG. 3 indicate that CCN5 administered by expression from viral vector AdCCN5 suppressed substantial restenosis, as indicated by comparison of the mid-panel and the right panel.

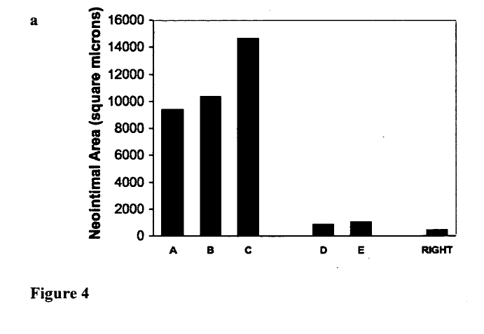

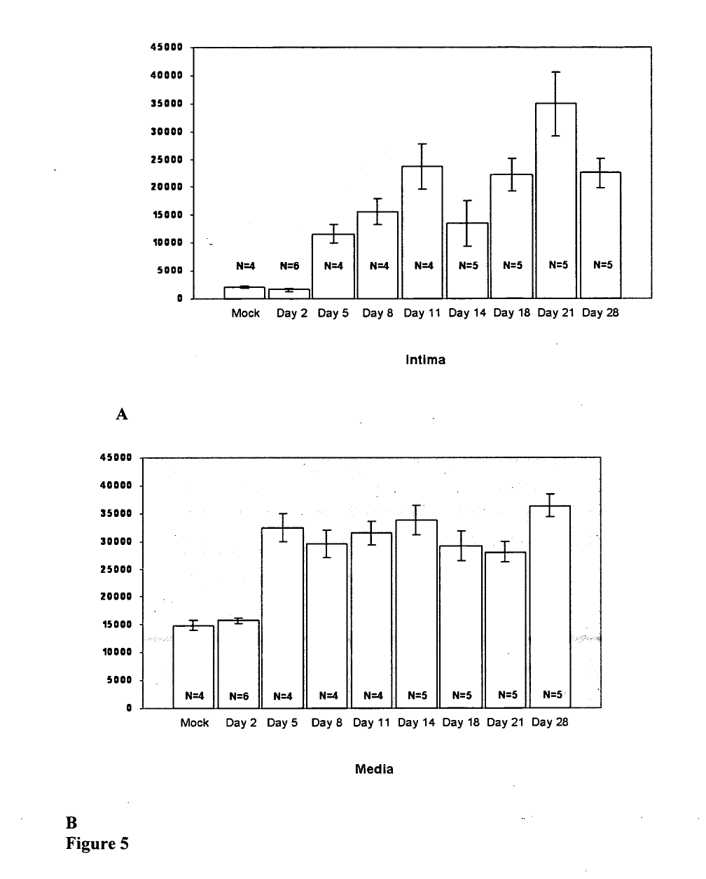

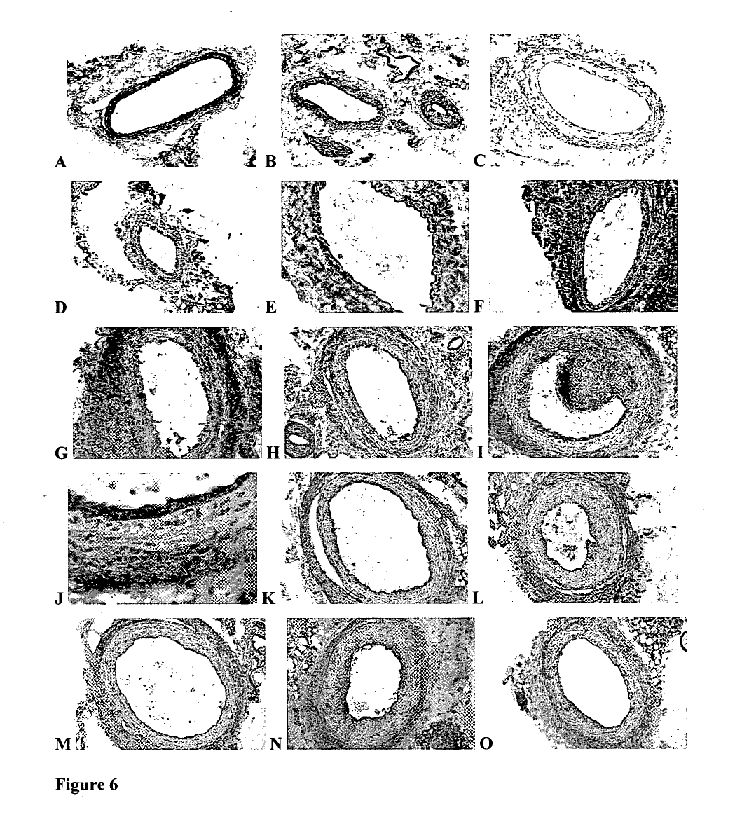

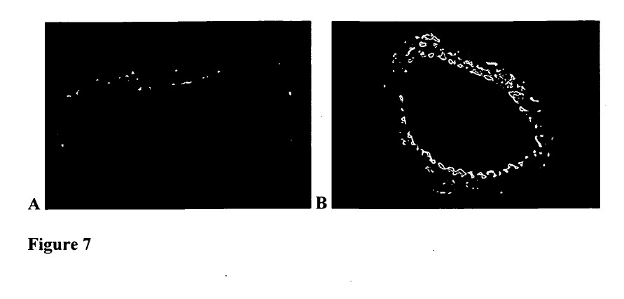

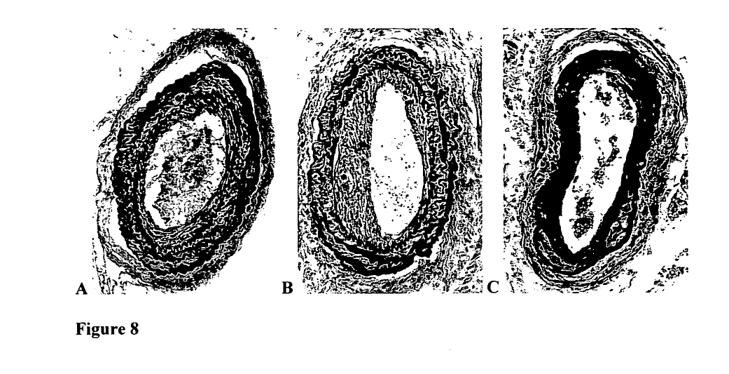

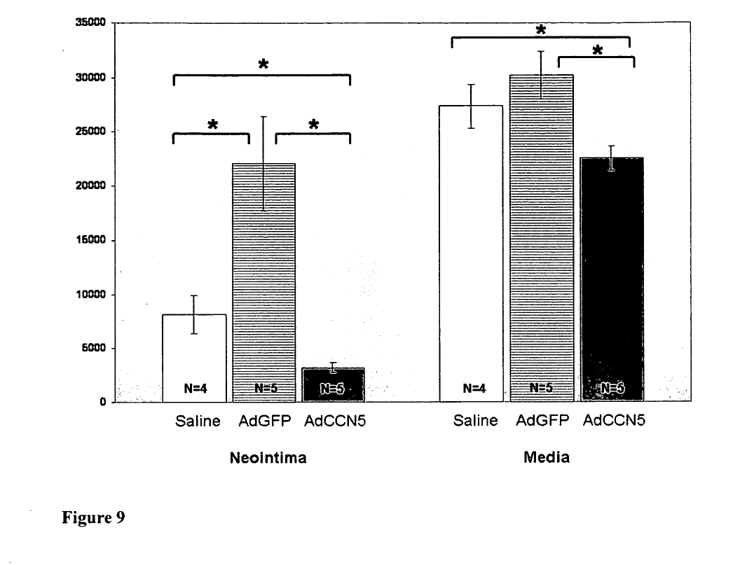

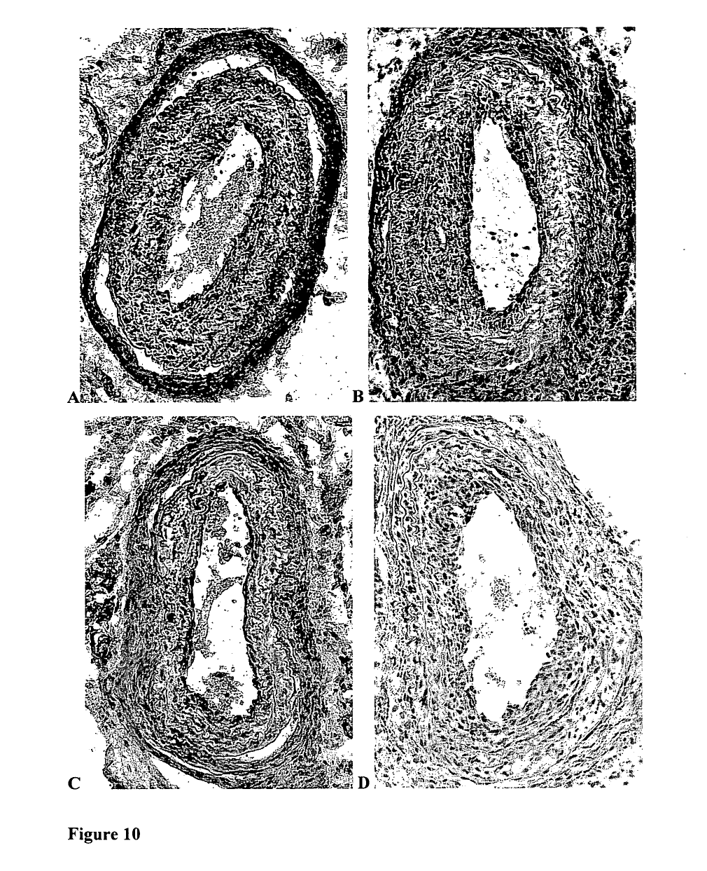

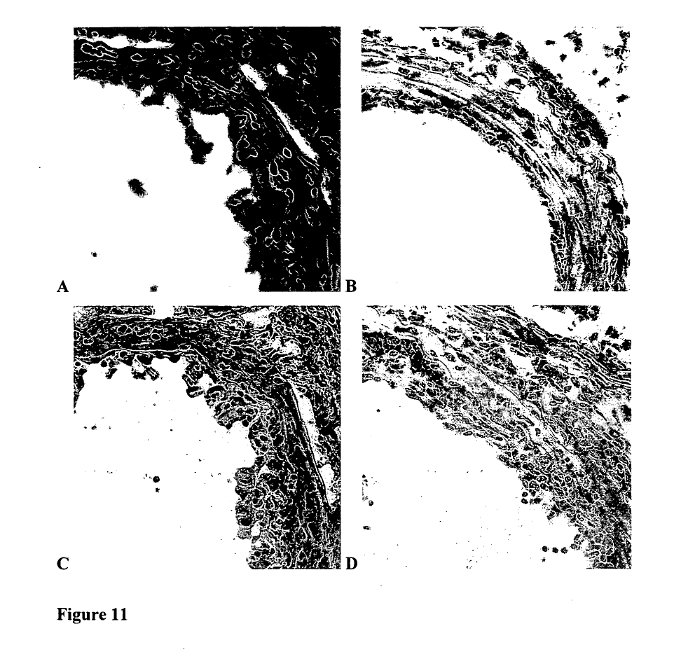

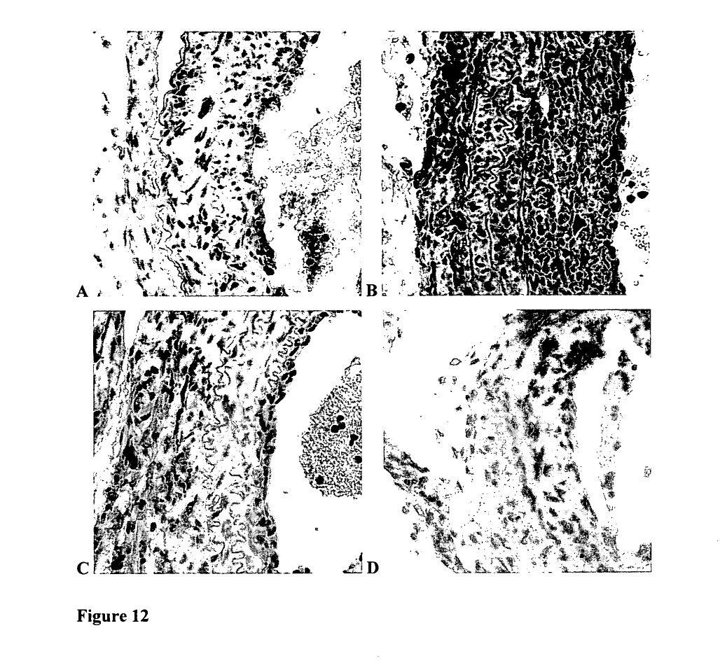

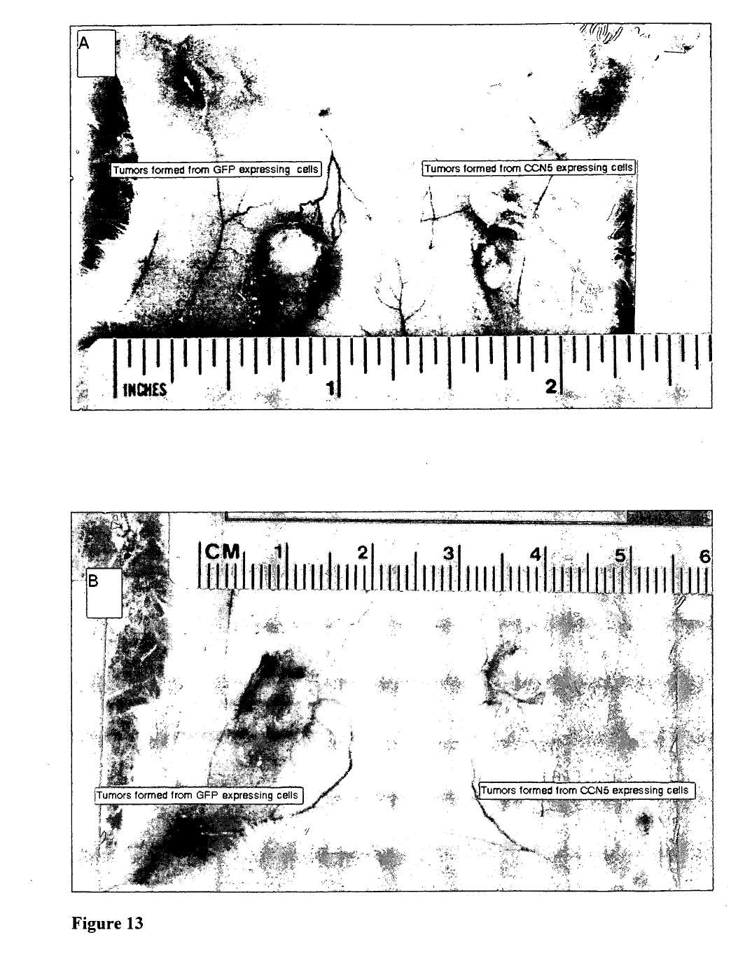

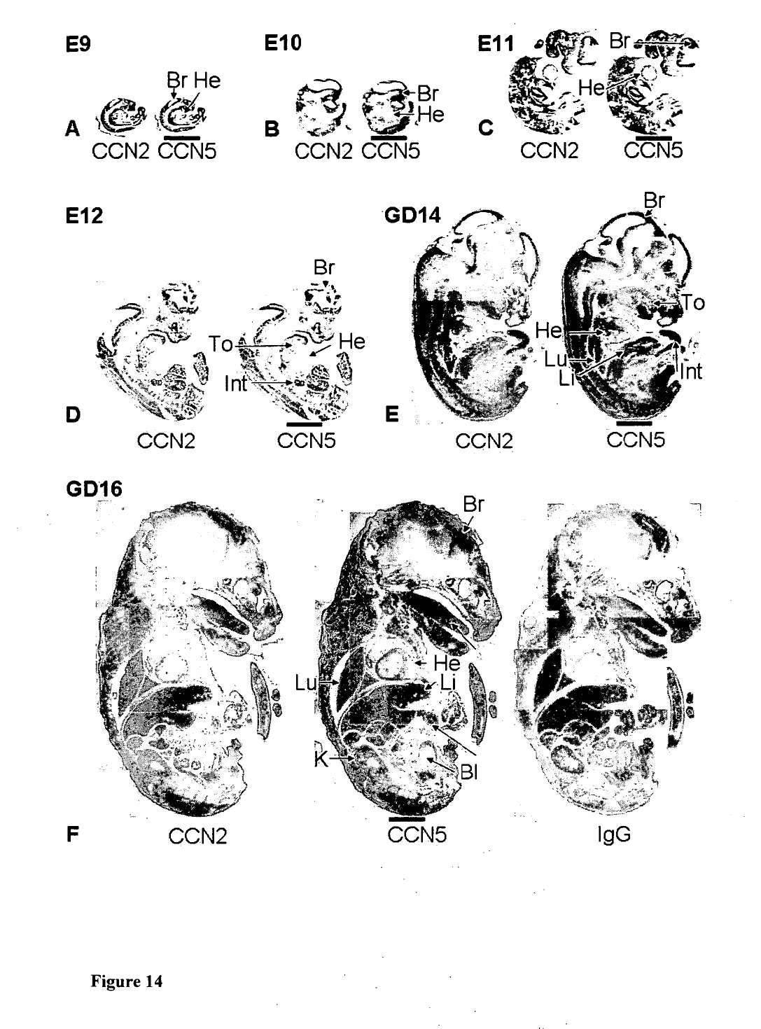

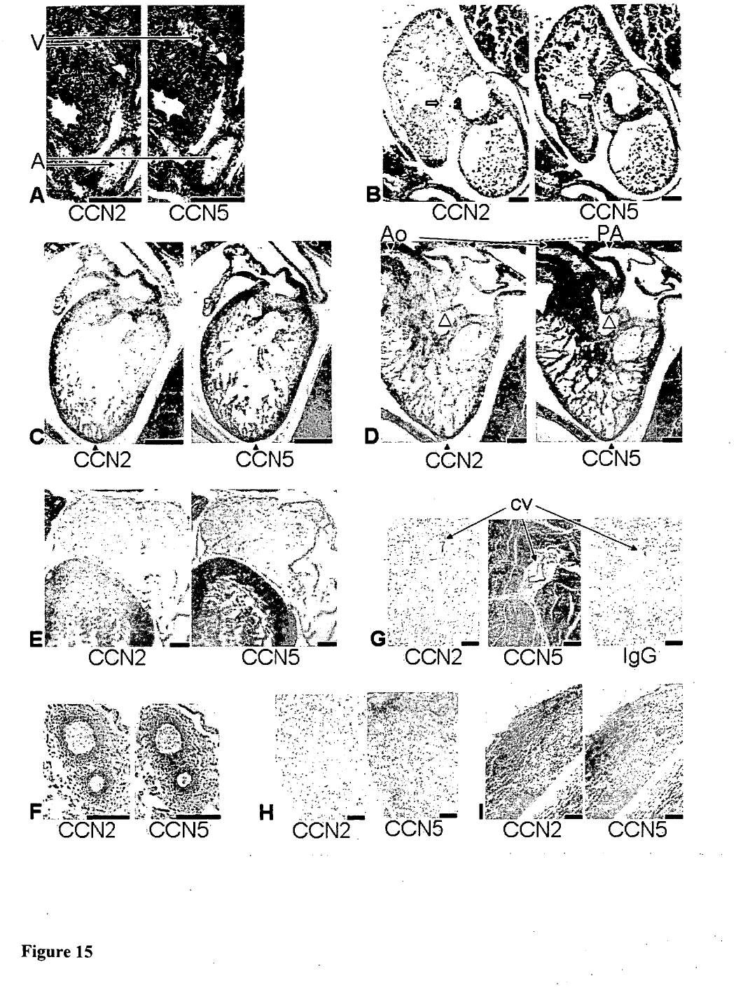

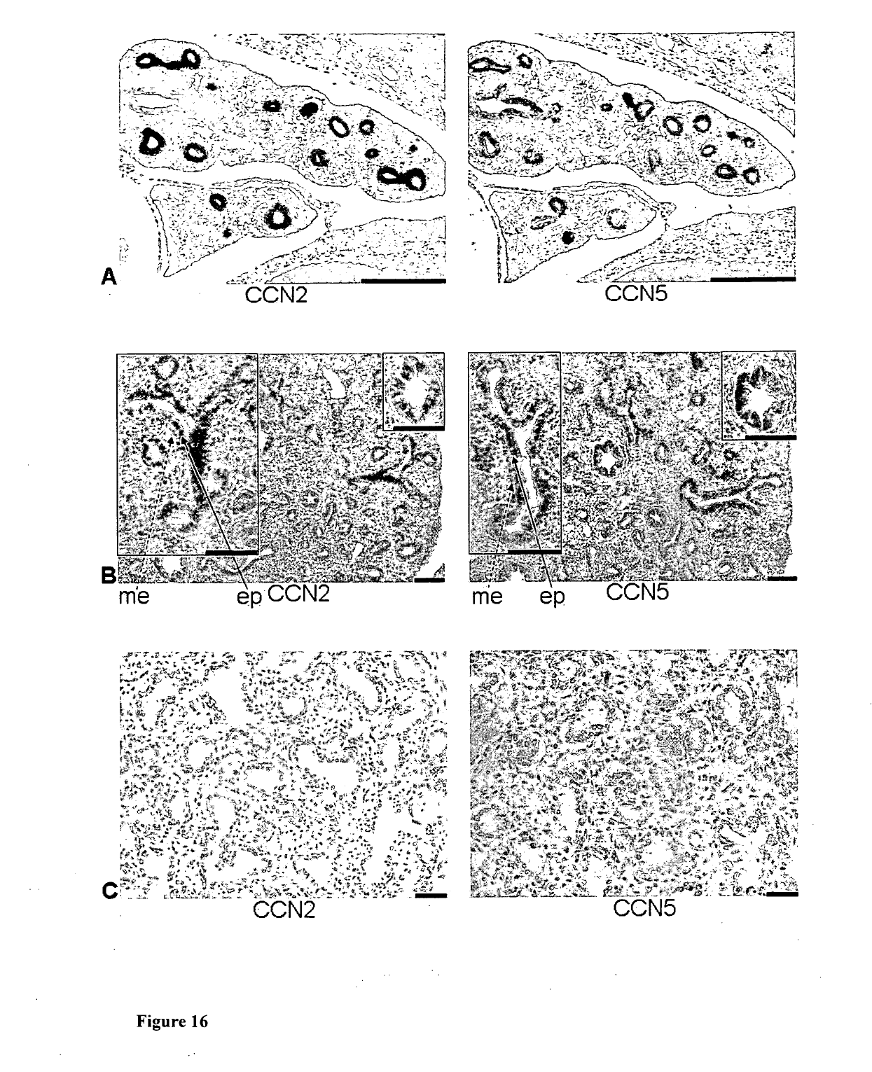

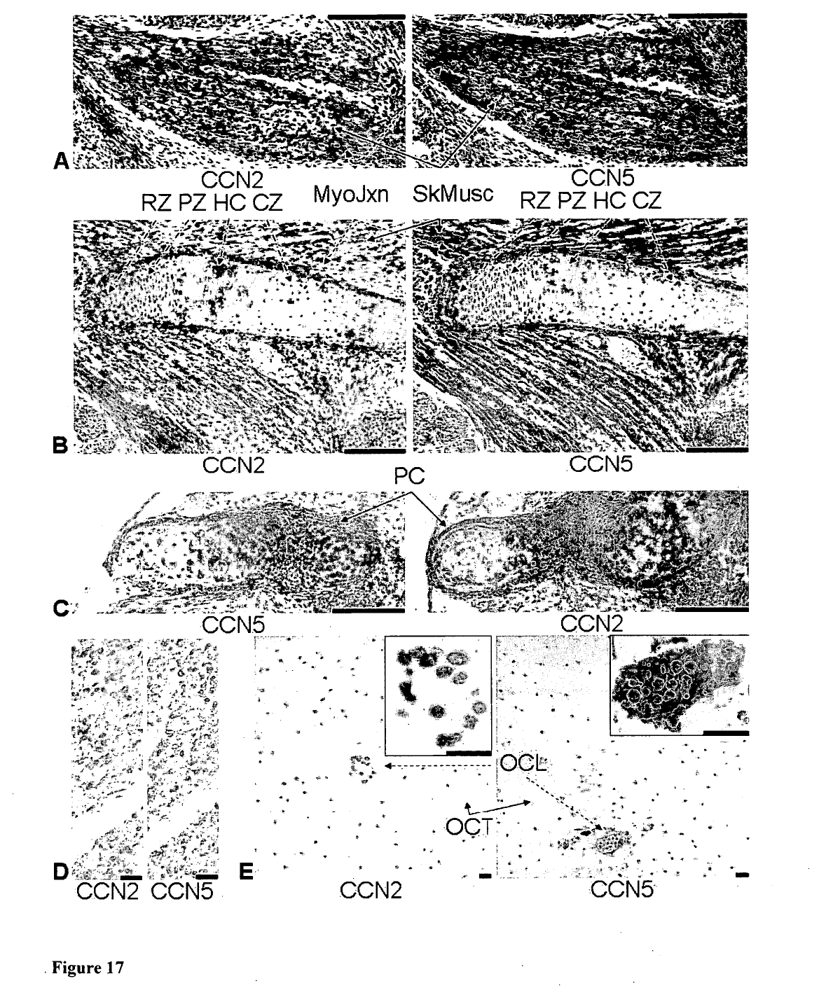

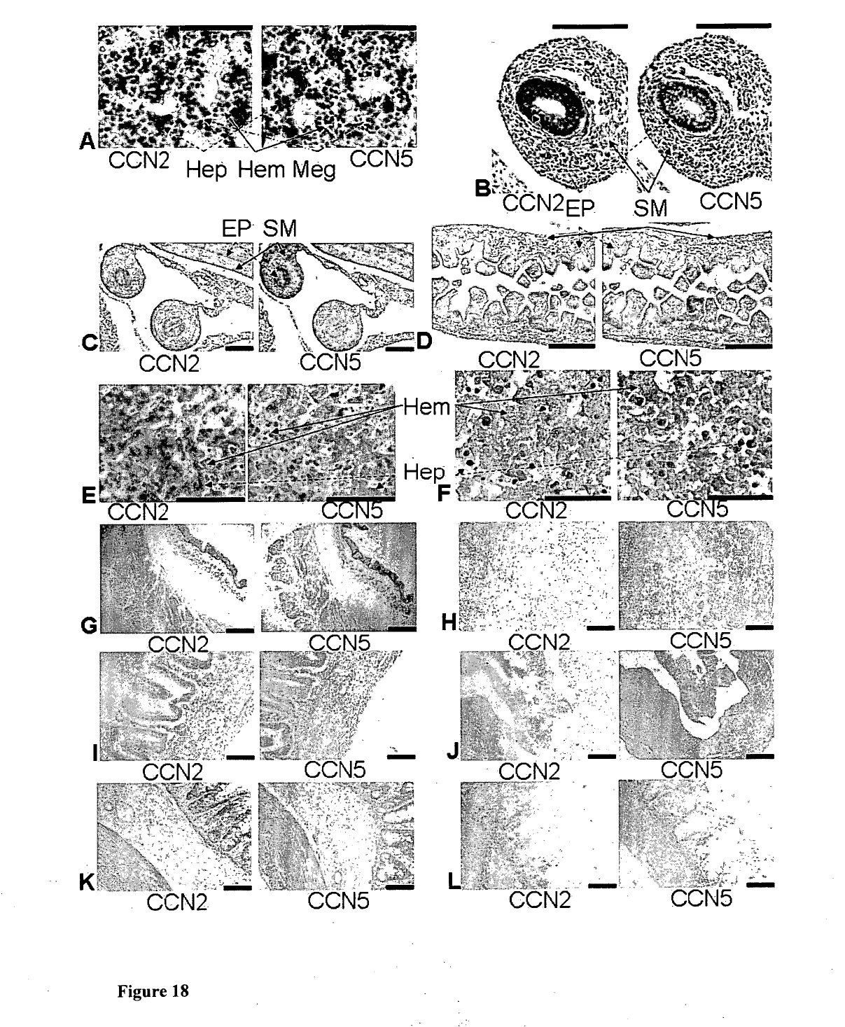

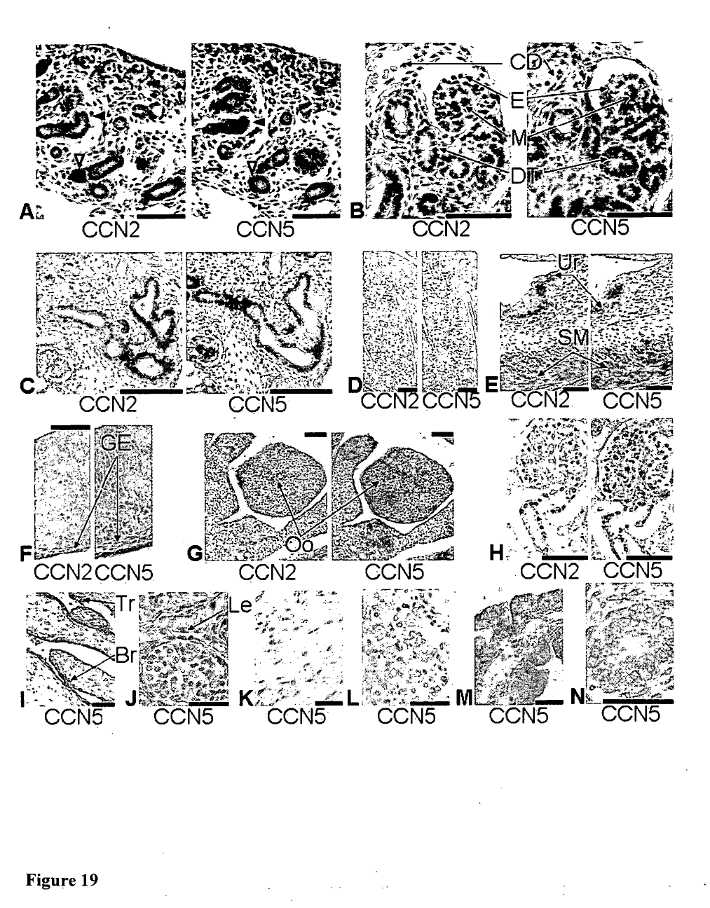

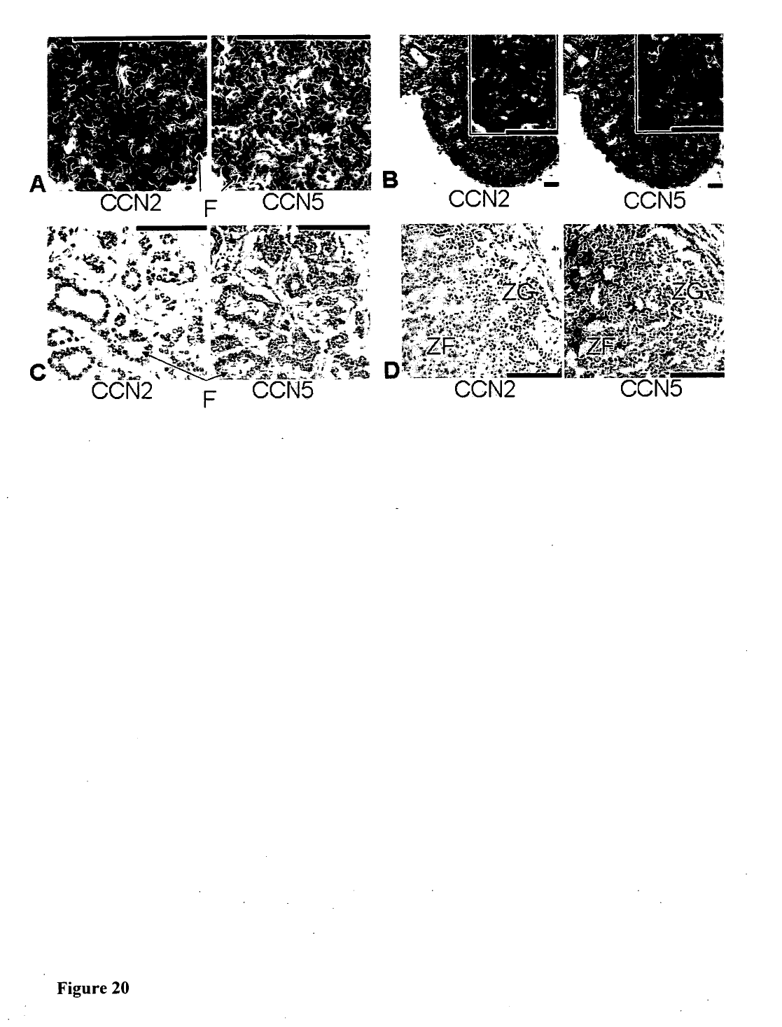

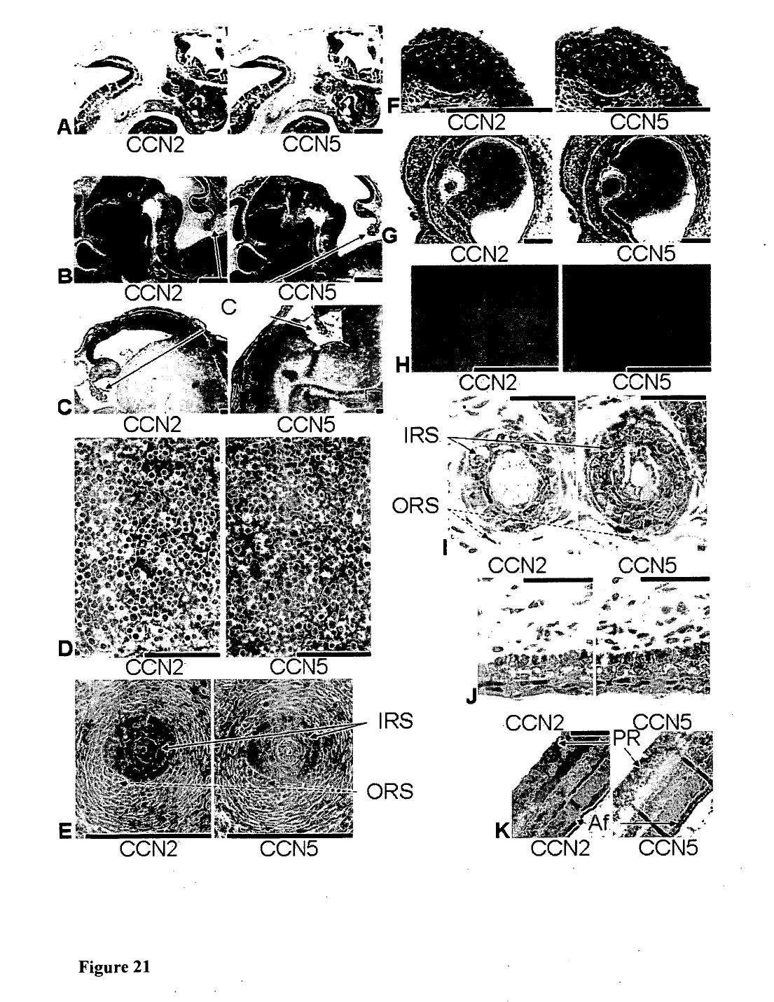

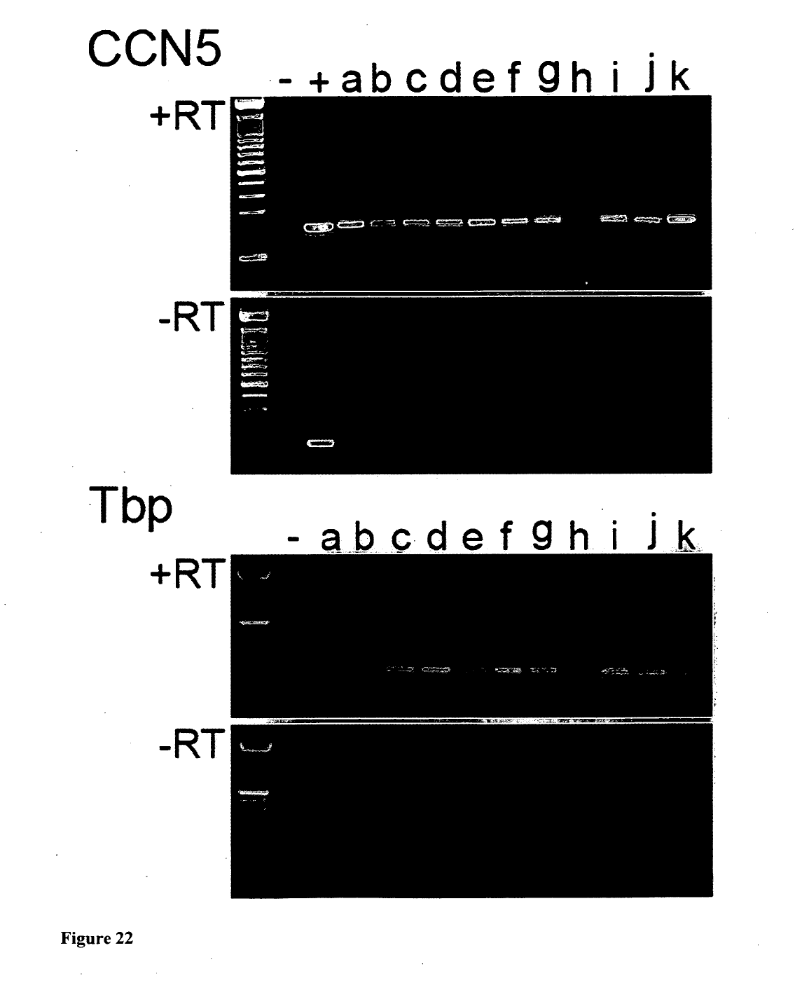

Quantitative morphometry of the arteries is shown in FIG. 4, to determine the neointimal areas of uninjured and injured arteries, injected either with control AdGFP, or administerd AdCCN5. The data show an unexpectedly large decrease in restenosis, i.e., 5-10 fold decrease in neointimal area in arteries administered CCN5. Further, a decrease in neo-intimal area was observed in AdCCN administered subjects (see FIG. 4 Panel A).

### Example 10

Carotid Ligation Increases Area of Vascular Media and Induces Neointimal Lesion

Male C57BL/6J 20-25 g mice received carotid ligation injury and were sacrificed for analysis between 2 and 28 days following injury. Carotid arteries were fixed, sectioned, and stained as described above.

No differences were observed in area of vascular intima (between IEL and lumen) between mock injured animals and animals examined 2 days following injury (FIG. 5 panel A). Beginning at day 2 and continuing through day 28 following injury it was observed that the neointima was increased relative to that of mock injured animals (p<0.05) or that observed at day 2. Neo-intimal area peaked at 21 days following injury. No difference in vascular media area (between EEL and IEL) was observed between animals receiving mock injury (examined 14 days following surgery) and animals receiving carotid ligation injury 2 days following surgery (FIG. 5 panel B). Beginning at day 5 and continuing through to day 28 following injury, vascular media increased approximately 2-fold relative to that in mock injured subjects, or day 2 following injury and remained elevated throughout the period of time examined (p<0.05).

### Example 11

Kinetics of CCN5 Expression Following Carotid Ligation Vascular Injury

The temporal and spatial expression pattern of CCN5 following mouse carotid ligation vascular injury was determined using a well-characterized anti-CCN5 antibody (Lake and Castellot, 2003; Gray et al., 2005; Gray et al., 2007; Lake et al., 2003; Malmquist et al., 2007; and Mason et al., 2004). CCN5 protein expression is high in vascular smooth muscle cells and endothelial cells of uninjured arteries, in agreement with previous studies that demonstrated CCN5 mRNA in cultured vascular smooth muscle and endothelial cells (Delmolino et al., 2001; Lake and Castellot, 2003), CCN5 protein in cultured vascular smooth muscle cells (Lake and Castellot, 2003; Lake et al., 2003), and CCN5 protein in uninjured mouse and rat aortic media and intima (Gray et al., 2005; Gray et al., 2007; Lake et al., 2003; and Malmquist et al., 2007). Negative controls exposed to PBS in place of primary antibody showed no brown staining in adventitia, media, or intima (FIG. 6 panel C).

CCN5 was found to be expressed at a moderate level in the arterial adventitia of uninjured (FIG. 6 panel A), mock injured (FIG. 6 panel B), and at day 2 following injury (FIG. 6 panel D). Beginning with 5 days following injury and continuing through 84 days (12 weeks) following injury, CCN5 expression was found to have increased in the tightly packed adventitial cells surrounding the injured artery (FIG. 6 panels E-O).

The data show that CCN5 was highly expressed in vascular media of mock injured (FIG. 6 panel B) and uninjured (FIG. 6 panel A) arteries, and most surprisingly, CCN5 expression in vascular media was greatly reduced 2 days following injury (FIG. 6 panel D). Medial CCN5 expression remained much lower than that seen in uninjured or mock injured arteries through 12 weeks following injury (FIG. 6 panels D-O). CCN5 is highly expressed in the endothelial cells of the intima in uninjured (FIG. 6 panel A), mock injured (FIG. 6 panel B), and day 2 following injury (FIG. 6 panel D). CCN5 expression remained present at reduced levels in neointima at days 5 and 8 following injury (FIG. 6 panels D-E), and began to increase throughout the lesion at day 11 through 12 weeks (FIG. 6 panels H-O) following injury.

### Example 12

CCN5 Gene Therapy Reduces Restenosis

To determine if CCN5 may be useful in reducing vascular injury, advantage was taken of a CCN5 expressing adenovirus (AdCCN5) that modulates proliferation, motility, and MMP-2 expression in cultured smooth muscle cells (Lake and Castellot, 2003; Lake, et al., 2003; Mason et al., 2002).

Animals were treated with AdCCN5 or a control vector (AdGFP). The mock injury animals were first ligated at the carotid artery and then AdCCN5 or a saline control was injected into an isolated arterial segment proximal to the ligation point. At a time point 25 minutes after the injection a second ligation was placed in order to close the hole made by the needle and restore blood flow to the region. Two days following these treatments, GFP fluorescence was observed throughout the intima and to a lesser degree in the vascular media of the treated carotid artery (FIG. 7 panel B), and was not observed in the contralateral untreated artery of the same animal (FIG. 7 panel A).

AdCCN5 treatment was observed to substantially reduce neointimal lesion formation in this carotid ligation injury model (FIGS. 8 and 9). Neointimal area in AdCCN5 treated animals was observed to be 2.5 fold less than that observed in negative control saline treated animals (p<0.05), while neointimal area in AdGFP treated animals was 2.7 fold greater than in saline treated animals (p<0.05). Observed medial area was not significantly different between animals administered saline or AdGFP treatment. While AdCCN5 decreased medial area by 1.2 fold or 1.3 fold compared to the saline control or AdGFP treatment, respectively (p<0.05), neointima in AdCCN5 treated animals was surprisingly reduced by 85% relative to AdGFP treated animals.

Because the treatment model involved each of placement of a clamp, intravascular injection and thereby extended surgical time, in comparison to the basic carotid ligation model, CCN5 expression was analyzed in comparison with that model. Data from animals treated intravascularly with saline, AdGFP, or AdCCN5 demonstrated that CCN5 expression 14 days following injury was increased in adventitia and neointima and was reduced in media in a pattern similar to that observed in animals studied using the carotid ligation model (FIG. 11 and FIG. 6 panel I).

### Example 13

Proliferating Cells of Vascular Media do not Express CCN5

To determine if reduction in CCN5 is generally found in proliferating cells as has been hypothesized from previous in vitro studies performed in animals injected with BrdU to label cycling cells at time points 12 and 1 hour(s) prior to sacrifice. Data show that only a small number of VSMC of the vascular media incorporated BrdU within the labeling period (FIG. 11 panel A). In contrast, CCN5 expression was globally reduced throughout the vascular media (FIG. 11 panel C). A similar pattern was observed in the media of adenovirus-treated animals treated with BrdU over a 14 day period (FIGS. 10 and 12). Successful BrdU incorporation and staining was verified by showing that intestinal epithelial cells were labeled. BrdU labeling of the adenovirus-treated animals demonstrates that a low rate of proliferation was occurring within the vascular media of AdCCN5 treated animals, with a higher degree of proliferation in AdGFP and saline treated animals. Neointimal proliferation was observed in the adventitia and neointima of all three groups (FIG. 12).

The expression pattern of CCN5 at day 2, 5, 8, 11, 14, 21, 28, 56, and 84 days following carotid ligation injury indicated high levels of CCN5 expression throughout mock injured or uninjured vascular media and endothelium. CCN5 expression was surprisingly reduced throughout the media two days following injury as the cells initially responded to the injury. As the injury progressed the CCN5 expression in these tissues remained reduced in the media up to 12 weeks following injury. As the neointima initiated formulation, CCN5 expression increased significantly in the adventitia and was present in the neointima, and surprisingly CCN5 was not observed to return to uninjured levels in the media. CCN5 gene therapy substantially reduced the size of the vascular lesion and was able to reduce restenosis in both the medial and neointimal vascular compartments in this model. These data show that CCN5 is an important down-regulator of VSMC proliferation, motility, and invasiveness in both normal and diseased arteries.

Medial area initiates an increase at day 5 following injury, and at day 14 following injury has reached a near-maximum injury. While neointima does not reach its maximum 14 days following injury, a significant increase is observed relative to unmanipulated arteries at this time point. Finally, endogenous expression in vascular media of CCN5 remains minimal 14 days following injury, and increases at low detectable levels at day 18 and later times following injury. Thus, exogenously supplied CCN5 expression is expected to affect injury kinetics at times points at day 14 following injury and earlier. Finally, as adenovirus reportedly expresses for up to two weeks following infection (Mitra et al., 2006) the role of CCN5 as a potential candidate for gene therapy was investigated at this time point.

CCN5 protein was highly expressed in endothelial cells of uninjured or mock injured vessels and at 2 days following injury. CCN5 is expressed at high levels in growth arrested endothelial cells, and is expressed at lower levels when cells are stimulated to proliferate in vitro (Delmolino et al., 2001). CCN5 was initially found at low levels in the developing neointima and was increasingly expressed as the lesion began to resolve. Recent evidence suggests that the neointimal cell population consists of a variety of different cell types, including primarily VSMC that have migrated out of the local vascular wall and proliferated and also adventitially derived myofibroblasts and bone-marrow derived progenitor cells (Bentzon et al., 2006; Hoofnagle et al., 2006; Forte et al., 2007).

The examples herein show that CCN5 is globally reduced throughout media (in both BrdU positive and negative cells when cells have been labeled acutely for only 12 hours or over the 14 day span of the injury). Examples herein show that, surprisingly, CCN5 is not reduced only in the context of an actively cycling VSMC in vivo, as was hypothesized from previous in vitro data Prior studies showed that while CCN5 mRNA and protein are high in cultured rat aortic SMC, that both mRNA and protein levels drop within 2 hours following serum addition (Delmolino et al., 2001; Lake et al., 2003). Medial VSMC CCN5 expression was shown herein to be lost throughout the media as early as 2 days following injury, at a time when <1% cells of the media were labeled with BrdU. Non-BrdU-labeled cells may be in a prolonged G1 phase; they are proliferating (not G0) even though they did not reach S phase within the labeling period. Reducing CCN5 in vitro through an siRNA technique increased VSMC motility, MMP-2 expression, reduced actin expression, and allowed cells to proliferate despite the presence of heparin, a strong inhibitor of VSMC proliferation (Lake and Castellot, 2003).

Thus, VSMC loss of CCN5 expression is associated with a phenotypic switch from a growth-arrested or quiescent state to a more active state. Without being limited by any particular theory or mechanism of action, CCN5 loss in VSMC indicates an “injured” or non-quiescent cell or tissue that is primed to enter the cell cycle due to a recent change in the local environment, perhaps independent of cell cycle status. Thus CCN5 protein and mRNA expression are here found to be present in developing mice at days 9-16 through gestation (Malmquist et al., 2007). CCN5 was expressed as expected due to previous observations in vascular smooth muscle and endothelium of day 16, but was also found expressed in a wide variety of cell and tissue types. While CCN5 expression was found in non-proliferating cell types as expected it was also widely expressed in rapidly proliferating tissues, suggesting that CCN5 expression is not strictly correlated with rapidly cycling cells.

CCN5 was herein found to be low in adventitia of uninjured vessels or at day 2 following injury, and was observed to increase at day 5 following injury. Adventitial proliferation is commonly observed in this and other models of vascular injury. The adventitia is thought to be comprised primarily of highly proliferative fibroblasts that differentiate into myofibroblasts. Adventitial cells may migrate through the vessel wall and contribute to neointimal formation (Bennett et al., 2001). CCN5 expression in proliferating mouse fibroblasts peaks during S-phase (Kumar et al. 1999).

AdGFP expression 2 days following injury was observed primarily in the endothelium and innermost layer of vascular media. Endogenous CCN5 expression was observed to be high in endothelial cells at day 2 and was no longer present in vascular media. Without being limited by any particular theory, the mechanism by which AdCCN5 transfection limits neointimal formation may involve expression within the innermost compartment of the media. The examples herein indicate that CCN5, whether delivered as DNA in a viral or plasmid construct or possibly as recombinant protein, is a good candidate for local delivery methods such as via catheter-driven methods or coated stents placed during or following a revascularization procedure (Mitra et al., 2006). Local delivery of CCN5 locally rather than increase of CCN5 systemically may be more effective and require lower dosages. Further, because of the widespread distribution in both embryonic and adult tissues (Delmolino et al., 2001; Gray et al., 2005; Gray et al., 2007; and Malmquist et al., 2007), local CCN5 may have fewer side effects.

While saline and AdGFP treated animals exhibited many proliferating cells in the media over the 14 day treatment period very few cells were labeled within the media of AdCCN5 treated animals. This suggests that AdCCN5 treatment limits medial expansion and neointimal formation through limiting proliferation of medial VSMCs. The observation of proliferation within the neointima of AdCCN5 treated cells further indicates that AdCCN5 treatment has not inhibited the regeneration of the endothelium following injury.

Inflammatory mediators have been implicated in neointimal hyperplasia (Scott, 2006; Schillinger et al., 2005; and Toutouzas et al., 2004). Adenoviral delivery may theoretically complicate treatment by increasing inflammatory mediators in the area following infection (Mitra et al., 2006; Smith et al., 2001). Because inflammatory mediators have been implicated in neointimal hyperplasia adenoviral delivery may hamper CCN5's ability to reduce hyperplasia. Data in examples herein did not show an increased infiltration of inflammatory cells such as lymphocytes or eosinophils in treated arteries at the adenovirus concentrations used at 14 days following injury. In contrast, to this result herein with CCN5, and in agreement with established models (Maillard et al., 2000), delivery of saline in place of adenovirus here resulted in a moderate increase in both medial and neointimal area when compared to adenoviral delivery of an anti-proliferative gene.

Most CCN proteins stimulate cell proliferation, in contrast to CCN5. The best understood CCN proteins—CN1 and CCN2—may be immediate-early serum responsive genes expressed by fibroblasts and other cell types (Brigstock, 2003; Perbal, 2004; and Gray and Castellot, 2004). They have been shown to stimulate cell proliferation (Frazier et al., 1996; and Kireeva et al., 1997), and there is evidence to suggest that the CT domain (absent in CCN5) is responsible for this activity in uterine SMC and fibroblasts (Ball et al., 1998; and Grotendorst et al., 2005). CCN1, CCN2, and CCN3 are low or not detected in uninjured arteries but increase in neointima following balloon angioplasty or in atherosclerosis (Ando et al., 2004; Cicha et al., 2005; Hilfiker et al., 2002; Schober et al., 2002; Ellis et al., 2000; and Grzeskiewicz et al., 2002). Although it has been hypothesized that CCN3 (NOV) might be a negative regulator of proliferation (Fukunaga-Kalabis et al., 2006), a direct assessment by cell counting methods has not been done. In fact, recombinant CCN3 was unable to inhibit proliferation of cells in culture, including SMC (Ellis et al., 2000b). Finally, over-expression of CCN2 has been shown to induce MMP2 (gelatinase A/type IV collagenase) in SMC (Fan, 2002). In contrast, CCN5 blocks MMP2 activity (Lake and Castellot, 2003).

Examples above were carried out in young, healthy animals. In the clinical situation, patients experiencing restenosis have had revascularization procedures performed to increase the luminal diameter of atherosclerotic, diseased vessels. Preliminary studies have indicated that CCN5 expression may be decreased in regions of plaque formation in atherosclerotic vessels. The results from examples herein showing that CCN5 delivery successfully inhibits restenosis in the carotid ligation model warrant further interest in the role of CCN5 in atherosclerosis and how it may be used in a therapeutic modality in those settings.

### Example 14

CCN5 Therapy for Human Breast Cancer Tumors

Data herein show that CCN5 modulates smooth muscle cell proliferation. Because tumor growth is associated with vascularization, it was here determined how CCN5 administered therapeutically might impact newly developing breast tumors, i.e., arising from single cells, in vivo in a mammal.

A sample of each of the adenovirus vectors expressing each of GFP and CCN5 under control of separate CMV promoters, or a strain expressing GFP under control of a CMV promoter, was administered (multiplicity of infection of 100/cell) to human mammary carcinoma cells (MDA-MB-231). The cells were incubated in culture for 48 hrs to allow expression of the adenoviral vector, and were then trypsinized and resuspended in Matrigel. To establish the human breast tumor model, one million cells in 250 ml of Matrigel were injected per site subcutaneously into the back of NOD/SCID mice. The mice were injected on the right side of the back with cells expressing CCN5, and injected on the left side of the back with cells expressing GFP.

The mice were sacrificed 80 days after injection, and the tumors were dissected and analyzed. As the tumors approximated ellipsoids in shape, quantitation was performed using a volume comparison, such that volume=4/3πr^{L}r^{W}r^{D} with L=Length, W=Width, and D=Depth. Table 1 below shows the results obtained from tumors from each group of animals.

TABLE 1
Tumor growth in NOD/SCID mice

| | GFP- | CCN5-expressing | | CCN5-expressing |
|---|---|---|---|---|
| | expressing | tumor mass | GFP-expressing tumor | tumor dimensions (cm) |
| Animal | tumor mass | (% of GFP) | dimensions (cm) | (% of GFP) |
| Mouse 1 | 0.385 g | 0.102 g | L = 1.17 W = 0.91 | L = 0.69 W = 0.35 |
| | | (26%) | D = 0.40 | D = 0.26 |
| | | | | (15%) |
| Mouse 2 | 0.398 g | 0.126 g | L = 1.34 W = 1.08 | L = 0.70 W = 0.68 |
| | | (32%) | D = 0.38 | D = 0.28 |
| | | | | (24%) |

The date in Table 1 show that tumors that had been formed from CCN5 expressing cells were observed to have a lower mass (less than one-third) and smaller dimensions (one fifth to one-quarter in volume) compared to tumors that had been formed from control GFP expressing cells. Further, the CCN5 expressing cells formed tumors that appeared less vascular as well as grossly smaller than from control GFP expressing cells (See FIG. 13, Panels A and B).

These data demonstrate that CCN5 administration slows growth of human breast cancer tumors in a mammalian system, NOD/SCID mice.

### Example 15

Expression of CCN5 During Embryonic Development

The temporospatial embryonic expression patterns of CCN1, CCN2, CCN3, and CCN4 have been analyzed in developing mammals (French et al, 2004; Friedrichsen et al, 2003; Ivkovic et al, 2003; Kireeva et al, 1997; Kocialkowski et al, 2001; Lopes et al, 2004; Natarajan et al, 2000; O'Brien and Lau, 1992; Surveyor et al, 1998; Surveyor and Brigstock, 1999). However, the expression pattern of CCN5 has not been explored during embryonic development. The strong anti-proliferative and anti-motility activity of CCN5 against cultured SMC predicts that CCN5 expression is low in early development, when cell proliferation and motility is high. CCN5 should increase in some embryonic tissues later in development when proliferation is complete and cells enter a more differentiated state. However, embryos develop in a very estrogen-rich environment, potentially inducing CCN5 expression in most or all embryonic tissues.

To resolve the alternative outcomes predicted by the anti-proliferative and estrogen-responsive nature of CCN5, we examined the expression patterns of CCN5 protein and mRNA and compared them to CCN2 protein expression patterns in developing mouse and human tissues.

The temporal and spatial expression pattern of CCN5 was determined during embryonic and fetal development using a well-characterized anti-CCN5 antibody (Lake et al, 2003; Mason et al, 2004a). Mouse embryos and fetuses were obtained at time points ranging from E9 to GD16 and prepared as described in Materials and Methods. Sections from human fetuses at 3 to 7 months of gestation were obtained and stained as described in Materials and Methods. The expression patterns of CCN5 were compared with those of CCN2, a prototypical CCN family member containing all four domains. CCN2 exhibits a biological activity profile that is almost completely opposite that of CCN5 in smooth muscle cells (Ando et al, 2004; De Falco et al, 2006; Fan et al, 2000; Fan and Karnovsky, 2002; Lake et al, 2003; Lake and Castellot, 2003; Mason et al, 2002). No color reaction was detected when pooled rabbit IgG was used in place of primary antibody.

Immunohistochemistry results demonstrate that CCN5 is widely expressed throughout early embryonic development of the mouse. The earliest time point examined was E9. CCN5 was found prominently expressed in tissues that originated from all three major embryonic germ layers (ectoderm, mesoderm, and endoderm; FIG. 14). CCN5 was ubiquitously expressed throughout the embryo at E9, E10, and E11. As organs developed further and distinct cell types differentiated at E12 and later times, some cell types demonstrated increased CCN5 expression and others demonstrated decreased CCN5 expression. CCN2 expression followed similar patterns of distribution and change. In general, CCN5 and CCN2 staining patterns were similar during E9-E11. Beginning at E12, staining differences were detected. CCN5 was present in a larger number of cell types, and CCN2 staining became more restricted and often less intense than that of CCN5 in GD14-16 mice. Exceptions to the general pattern include lung and intestine, in which GD14 branching bronchioles and E12 intestinal epithelium expressed CCN2 more than CCN5.

In the examples below, comparisons of CCN5 and CCN2 expression patterns in mouse and human tissues of the major organ systems at several time points in embryonic and fetal development are shown. We observed widespread expression of CCN5 early in development that becomes increasingly restricted close to parturition. These findings have important implications for the biologic and physiologic roles of CCN5.

### Example 16

CCN5 Expression in the Cardiovascular System

The cardiovascular system is the first functional organ system in developing mammalian embryos. Mouse CCN5 protein was detected throughout the myocardium and large vessels, and CCN2 was detected at lower levels than CCN5 (FIG. 15). Mouse CCN5 was highly expressed in the endothelium and smooth muscle of veins and arteries, and CCN2 was present at moderate levels (FIG. 15 Panel A). High levels of both CCN2 and CCN5 were detected in the apex of the developing mouse heart during E12-GD14. At GD16, CCN5 expression in myocardium was uniform and not increased at the apex. By GD16, CCN2 expression was decreased throughout the myocardium compared to CCN5, and was not concentrated at the apex (FIG. 15 Panels B-E). At E12, mouse CCN5 was present at lower levels in the forming atrioventricular septum, when the remaining myocardium demonstrated uniform levels of CCN5 (FIG. 15 Panel B). CCN5 levels were very low and CCN2 was absent in the developing cardiac valves (FIG. 15 Panel D). At GD14, mouse CCN5 levels were lower in the thick-walled aorta when compared to the thinner-walled pulmonary arterial trunk (FIG. 15 Panel D). Mouse umbilical vessels expressed CCN5 and a lower level of CCN2 (FIG. 15 Panel E).

Human CCN5 was detected throughout the human fetal myocardium, endothelium and smooth muscle of coronary arteries and veins and to a lesser extent in other large vessels at 4 months of gestation (FIG. 15 Panels G-H). CCN2 expression was markedly lower than that of CCN5 in fetal cardiovascular tissues, similar to the previously described mouse observations. The smooth muscle and endothelium of human umbilical vessels displayed a high level of CCN2 and a moderate level of CCN5 (FIG. 15 Panel I).

### Example 17

CCN5 Expression in the Respiratory System

Mouse lung development between GD14-GD16 is considered the pseudoglandular period, corresponding to weeks 5-17 in human development (Van Tuyl and Post, 2003; Volpe et al, 2003). In GD14 and GD16 mouse fetuses, CCN5 was present uniformly in both the epithelial and mesenchymal cells surrounding branching bronchioles (FIG. 16 Panels A-B). In contrast, CCN2 was present only in the bronchiolar epithelium and not in the surrounding mesenchyme. Some terminal ends of actively branching bronchioles displayed very high levels of CCN2 at GD14 (FIG. 16 Panel A). At GD16, some bronchiolar terminal ends demonstrated little or no CCN2 and others expressed higher levels of CCN2, while all bronchiolar terminal ends displayed abundant CCN5 staining (FIG. 16 Panel B). CCN5 was present in the epithelial and mesenchymal cell layers of larger bronchioles, and CCN2 was absent (FIG. 16 Panel B). CCN2 was present mostly in actively growing and branching bronchi, but not in some terminal ends of actively branching bronchioles and in the bronchiolar epithelium after, larger bronchioles are established. In contrast, CCN5 was prominent in the larger bronchioles and was expressed throughout actively branching bronchioles.

Human fetal lung tissue at 5 months of development demonstrated CCN5 at moderate uniform levels in epithelial and mesenchymal cells, and no CCN2 staining (FIG. 16 Panel C). At this time in development (the canalicular period), the lung has finished forming the bronchial tree and is forming the acini, capillary network, and alveolar type I and II cells (Wert, 2004).

### Example 18

CCN5 Expression in the Musculoskeletal System

High levels of mouse CCN5 were detected in GD14-16 mouse skeletal muscle (FIG. 17 Panels A-B). CCN5 staining was particularly strong at myotendinous junctions, beginning in GD14 and becoming pronounced by GD16. In contrast. GD14-16 skeletal muscle displayed very little CCN2 and it did not accumulate at myotendinous junctions. Both CCN2 and CCN5 staining was prominent in chondrocytes during endochondral ossification (FIG. 17 Panels B-C). Hyaline cartilage in the resting zones displayed neither CCN2 nor CCN5. CCN5 was present in the chondrocytes of the proliferating, hypertrophic, and calcification zones, and CCN2 was seen primarily in the chondrocytes of the hypertrophic and calcification zones. In addition, perichondrial and periosteal cells demonstrated high levels of CCN5, as well as some CCN2 (FIG. 17 Panel C).

In the human fetus at 5 months of gestation, CCN5 expression was moderate and CCN2 was detected at a low level in skeletal muscle (FIG. 17 Panel D). The human fetal samples analyzed did not include myotendinous junctions. CCN2 was not detected in human fetal osteocytes or osteoclasts (FIG. 17 Panel E). In contrast, CCN5 staining was prominent in osteoclast nuclei and absent in osteocytes (FIG. 17 Panel E).

### Example 19

CCN5 Expression in the Gastrointestinal System

In GD14-16 mouse liver, both CCN2 and CCN5 staining were detected in hepatocytes but not hematopoietic stem cells (FIG. 18 Panel A). Megakaryocytes express CCN5 but not CCN2.

The developing small intestine of the mouse revealed high levels of both CCN2 and C epithelium, and then much weaker by GD16 (FIG. 18 Panel D). CCN2 staining was very low in intestinal smooth muscle throughout all of the time points examined. CCN5 was detected at lower levels in intestinal smooth muscle and epithelium beginning at E12 (FIG. 18 Panel B) and GD14 (FIG. 18 Panel C), and at higher levels in GD16 fetuses (FIG. 18 Panel D).

Human fetal hepatocytes at 4 months of development revealed slightly more CCN2 staining than that of CCN5 (FIG. 18 Panel E). However, at 5 months of gestation, fetal hepatocytes revealed low levels of CCN2 and much higher levels of CCN5 (FIG. 18 Panel F). Liver hematopoietic stem cells do not express CCN5 or CCN2.

Analysis of CCN2 and CCN5 protein expression patterns in the developing human intestinal tract revealed a gradation of expression among different sections of the digestive tract. CCN2 and CCN5 were present at the same levels in the esophagus at 6 months (FIG. 18 Panel G); in this structure, levels of both proteins were high in the muscularis mucosae and muscularis extema. CCN2 and CCN5 levels were also high in the esophageal epithelium, and absent in the lamina propria. Other regions of the human fetal intestine were examined at 5 months. CCN5 was detected in the stomach musculature and at lower levels in the epithelium and lamina propria at 5 months; CCN2 staining was absent in the stomach (FIG. 18 Panel H). CCN5 staining, but not that of CCN2, was detected in the smooth muscle and intestinal epithelium of the small intestine at 5 months (FIG. 18 Panel I). CCN5 staining was present uniformly throughout the gallbladder smooth muscle and epithelium, and CCN2 staining was absent (FIG. 18 Panel J). CCN5 staining was detected in the smooth muscle of the colon (FIG. 18 Panel K). CCN2 was absent in the colon except for the first few layers of intestinal smooth muscle that face the lumen. Both CCN2 and CCN5 were absent in the base of the intestinal crypts (the location of proliferating colonic epithelium) and strongly present in the tips of the villi (the cells that have moved away from the crypts and are no longer proliferating). CCN5 was detected at very low levels in the colon lamina propria, and CCN2 was absent (FIG. 18 Panel K). In the rectum, CCN5 was detected at low levels in smooth muscle, lamina propria, and non-proliferating epithelium; CCN2 was not present in these tissues (FIG. 18 Panel L).

### Example 20

CCN5 Expression in the Kidney and Urogenital System

In early mouse kidney development (E12), mesonephric tubules display higher levels CCN5 than that of CCN2 (FIG. 19 Panel A), preferentially overexpressing one protein and downregulating the other. CCN5 was prominent in glomerular mesangial cells, tubules, blood vessels, and collecting ducts, while CCN2 was detected at high levels only in tubules at GD16 (FIG. 19 Panels B-C). Glomerular endothelial cells do not display any CCN5, in contrast to endothelial cells of larger vessels. GD14 mouse bladder smooth muscle, mesenchyme, and urothelium displays CCN5 and but CCN2 was only detected at low levels in the urothelium (FIG. 19 Panel D). In the mouse bladder at GD16, the urothelium and smooth muscle display CCN2 and high levels of CCN5 (FIG. 19 Panel E).

The germinal epithelium and ovary reveal CCN5 staining at GD14 (FIG. 19 Panel F). Higher levels of CCN5 staining and low levels of CCN2 staining were found in the ovary and epithelial cells and glands of the reproductive tract at GD16 (FIG. 19 Panel G).

In the human fetal kidney at 5 months, CCN5 staining was detected in mesangial cells, tubules, and blood vessels (FIG. 19 Panel H). CCN2 was present only in some tubules (FIG. 19 Panel H).

Low levels of CCN5 staining were detected in the developing human reproductive system at 5 months, except for the very high levels in placental trophoblasts (FIG. 19 Panel I). CCN5 staining was absent in regions where the chorionic villi fused together and branched. Weak CCN5 staining was detected in testicular Leydig cells (FIG. 19 Panel J), uterus (FIG. 19 Panel K), ovarian stroma (FIG. 19 Panel L), fallopian tube (FIG. 19 Panel M), and epididymis (FIG. 19 Panel N). In contrast, CCN2 was absent in all developing human reproductive organs examined.

### Example 21

CCN5 Expression of Endocrine and Immune Organs

In both mouse and human thyroid and adrenal glands, moderate CCN5 levels were detected in some cell types, while CCN2 expression was either very low or undetectable (FIG. 20).

In GD16 mouse (FIG. 20 Panel A), very low levels of CCN2 and moderate levels of CCN5 were observed. CCN5 staining was detected on the colloidal side of developing thyroid follicles, suggesting that the thyroid follicle cells produce and excrete CCN5. CCN5 was detected in mouse GD16 adrenal gland but CCN2 levels were very low (FIG. 20 Panel B). Both CCN5 and CCN2 were detected in the connective tissue of the adrenal capsule. CCN5 staining was observed in the nuclei of some adrenal cells (FIG. 20 Panel B, inset). The pancreas in developing mice displayed low levels of CCN5 and CCN2 in the exocrine ducts. CCN2 and CCN5 levels were extremely low or absent in thymus and spleen.

In 5 month human fetal thyroid, (FIG. 20 Panel C), moderate levels of CCN5 were observed, and CCN2 was not detectable. In the 5 month human fetal adrenal gland, CCN5 was prominent in the zona fasciculata, the region of the gland that makes cortisol, and very low in the zona glomerulosa, the region that produces aldosterone (FIG. 20 Panel D). No CCN2 staining was observed in the human fetal adrenal gland (FIG. 20 Panel D). Human fetal pancreas demonstrated only CCN5, and not CCN2, in the exocrine ducts. Very little CCN2 or CCN5 was detected in fetal human thymus and spleen.

### Example 22

CCN5 Expression in Nervous System and Skin

Both CCN2 and CCN5 were widely distributed in the brain of E12 mouse embryos (FIG. 21 Panel A). Expression of both proteins was much more restricted in the later GD14 (FIG. 21 Panel B) and GD16 fetuses (FIG. 21 Panel C). Many cells in the developing mouse brain and spinal cord demonstrated CCN2 and CCN5 predominantly and strongly in the nucleus of neurons (FIG. 21 Panel D), in contrast to most cell types where CCN5 is found primarily in cytoplasm or tightly bound to the outer surface of the cell membrane. Both CCN5 and CCN2 were present in the choroid plexi, particularly at GD16 (FIG. 21 Panels B-C).

Both CCN2 and CCN5 were present in the developing GD16 mouse vibrissae (FIG. 21 Panel E). CCN5 was observed in both the inner and outer root sheath layer of cells, and CCN2 was limited to the inner sheath. Both CCN2 and CCN5 were detected a high levels in the developing epidermis of GD16 mouse (FIG. 21 Panel F).

In the mouse eye at GD16, CCN5 and CCN2 were detected in the fused eyelid and corneal epithelium (FIG. 21 Panel G). CCN5, and not CCN2, was detected in the mouse corneal stroma.

Neither CCN5 nor CCN2 was found in the human fetal brain at 4 months (FIG. 21 Panel H). This result may be due to species differences in expression of this protein in brain between mouse and human. Alternatively, because only cores of tissue were examined it is possible that CCN2 or CCN5 was present in the developing human brain in other regions, or at other time points not examined in this study.

As we observed in the corresponding mouse tissues, both CCN2 and CCN5 were present in human fetal hair follicles at 5 months (FIG. 21 Panel I), with CCN5 found in both the inner and outer root sheath layer of cells and CCN2 limited to the inner sheath. Both CCN2 and CCN5 were detected a high levels in the developing epidermis of 5 month human fetal skin (FIG. 21 Panel J).

In the human fetal eye at 4 months, the rod/cone photoreceptor segment of the retina displayed high levels of CCN5, but not CCN2 (FIG. 21 Panel K). Both CCN2 and CCN5 were detected in the afferent nerve fibers.

### Example 23

Fetal Expression of CCN5 mRNA

Immunohistochemical analysis provides the most accurate representation of the location of the CCN5 protein. It is possible that this protein is translated, secreted, and then distributed to target cells and tissues. The sites of CCN5 protein translation and final distribution were compared by analyzing mouse fetal tissues for CCN5 mRNA. Organs from GD14.5 mice were collected and mRNA prepared for analysis by RT-PCR. CCN5 mRNA was found in every fetal organ examined, including lung, limbs and tails, umbilical cord, intestine, heart, liver, carcass, and head (FIG. 22). CCN5 mRNA was also demonstrated in the maternal placenta. We previously demonstrated CCN5 mRNA in the uterus and aorta (Delmolino et al, 2001; Mason et al, 2004a). These results suggest that the observed immunohistochemical findings in GD14 mice are the result of local synthesis of CCN5 mRNA in all of the tissues examined. Although RTPCR results are semi-quantitative, levels of CCN5 mRNA correlated with CCN5 staining intensity. For example, the umbilical cord produced high levels of CCN5 mRNA, and mRNA levels in the liver were low, consistent with our IHC data (FIG. 15 Panel H and FIG. 8 Panel A). CCN5 protein levels correlated with CCN5 mRNA levels, suggesting that secreted CCN5 remains associated with cells that produced it.

In this example, the distribution pattern of CCN5 protein during embryonic and fetal development of the mouse was comprehensively examined, and compared to the expression pattern in human fetal tissues. The temporospatial localization of CCN5 protein was compared with that of CCN2. These two CCN family members have been postulated to have opposite or complementary biological functions. Immunohistochemical data was compared with CCN5 mRNA levels in selected organs and time points in the GD14.5 mouse, and compared with the currently available data from similarly comprehensive studies on developing mammalian mRNA and protein distribution for other CCN family members.

The strong anti-proliferative and anti-motility activity of CCN5 against cultured SMC (Delmolino et al, 2001; Lake et al, 2003; Mason et al, 2004b) predicted that CCN5 expression would be low in early development when cell proliferation and motility is high, and CCN5 expression would increase in embryonic tissues later in development as cells stopped proliferating and entered a more differentiated state. On the other hand, embryos develop in a very estrogen-rich environment, and CCN5 is strongly induced by estrogen (Banerjee et al, 2003; Fritah et al, 2006; Gray and Castellot, 2005; Inadera et al, 2000; Mason et al, 2004a; Mason et al, 2004b), thus predicting that CCN5 would be present in most or all embryonic tissues in response to this stimulus. In general, CCN5 protein was found in the three major germ layers at all embryonic stages analyzed. CCN5 is present in most or all cells in early embryos, and then tissue-specific CCN5 expression differences appear as embryos age. CCN5 expression was not restricted to cell types of a particular embryonic lineage.

There are several discrepancies among published reports of CCN2 protein or mRNA expression that remain unresolved. Discrepancies between ISH and IHC data can occur for a number of reasons, including post-transcriptional modifications that alter mRNA translation or stability, and post-translational processing that alters protein maturation, accumulation, or degradation. Discrepancies in mRNA or protein distribution may be due to differences in sensitivity of the methods. For example, Kireeva et al (1997) used a peroxidase-conjugated secondary antibody and found less CCN2 in many organ systems than examples herein, and Surveyor and Brigstock (1999) using the more sensitive avidin-biotin technique. Discrepancies may also be due to differences in the affinity, avidity, or epitope availability of the specific CCN2 antibodies used, as well in the specific fixation techniques used. CCN2 distribution observed herein was mapped with an antibody against amino acids 223-348 of the CCN2 protein, while previous efforts employed antibodies against amino acids 165-200 (Kireeva et al, 1997) and amino acids 80-93 (Surveyor and Brigstock, 1999) of the CCN2 protein. This latter antibody was also used and the same CCN2 expression pattern with the aa223-348 antibody was observed when using identical tissues and fixation conditions.

It was generally observed from examples herein that while CCN2 has a more restricted expression pattern than CCN5, many developing cell types and tissues express both proteins. With a few exceptions, cell types expressing CCN2 also express CCN5. However, CCN5 was highly expressed in many cells where CCN2 expression was low or absent, for example in developing skeletal muscle myotendinous junctions. In conclusion, while in certain disease systems CCN2 increases in pathologies where CCN5 decreases, this pattern is not generally true for developing human and mouse tissues.

In contrast to predicted CCN5 levels that would be low in developing vascular smooth muscle because the embryo is actively growing, it was observed herein that CCN5 levels remained high, suggesting that the carefully controlled cell proliferation required for embryonic growth does not require an environment with low CCN5 levels. Lower CCN5 expression in aorta relative to that seen in the pulmonary artery was observed. Because aortic wall smooth muscle is exposed to higher mechanical pressure, lower levels of CCN5 may permit more remodelling and consequent thickening of the aortic wall to accommodate the increased pressure and flow requirements of the growing fetus.

The presence of CCN5 in mesenchymal cells of developing lung suggests that it may be important for signaling epithelial growth, such as defining barriers or edges of the bronchiole. Although CCN5 was highly expressed in both mesenchymal and epithelial cells of the developing airway, CCN2 was observed only in the epithelial cells. The interactions between epithelial and mesenchymal cells are critical in airway formation and differentiation (Evans et al, 1999; Van Tuyl and Post, 2003). In addition, CCN5 was present at high levels in the larger bronchi, and no CCN2 was present. The primary bronchioles and major airways form earlier in development and are thus relatively quiescent at this stage (Van Tuyl and Post, 2003). The presence of high levels of CCN2 in some terminal bronchiolar buds but not others suggests that CCN2 might be required only in actively branching bronchioles, and the continued presence of CCN5 throughout both actively branching and stable bronchioles suggests it might be required throughout development of these structures.

In the musculoskeletal system, the pattern of integrin αv protein expression is concentrated at the extremities of apical myotubes marking their shape (Hirsch et al, 1994; Tarone et al, 2000). The pattern of CCN5 expression observed herein was similar, thus CCN5 may participate in the organization of the myotendinous junction, and integrin αv may be important for CCN5 signaling.

In addition to CCN2 and CCN5, other CCN proteins have also been detected in bone. For example, CCN3 is expressed in normal osteoclasts (Manara et al, 2002), and CCN1 and CCN4 (French et al, 2004; Kireeva et al, 1997; O'Brien and Lau, 1992). In contrast to previous observations in 2 week old rats, we did not detect CCN2 in osteocytes, suggesting that the role of CCN2 in this cell type may not emerge until bones have fully formed (Safadi et al, 2003). The presence of CCN5 in the proliferating and calcification zones of ossifying bones suggests that it has an important role in ossification. The expression pattern we observe is similar to that reported by Kumar et al (1999). The opposite expression patterns of CCN2 and CCN5 in hypertrophic chondrocytes suggest that these proteins may work in concert to regulate ossification.

A change from patchy to uniform CCN5 expression was observed in the liver as gestational age increased. This may reflect the change of liver function as a hematopoietic organ in younger fetuses to a non-hematopoietic organ in 5 month old fetuses (Tavian and Peault, 2005). At GD16 in mice, approximately 40% of the liver volume is dedicated to hematopoiesis (Dzierzak and Medvinsky, 1995). Liver hematopoiesis remains important throughout mouse fetal development and is present at much lower levels in the 5 month human fetus.

Because of previous studies of differential expression of CCN5 relative to growth state of the cell, it was expected that rapidly proliferating cells of intestinal smooth muscle would not express high levels of CCN5, and quiescent or growth-arrested cells would express large amounts of CCN5. This pattern is present in the colonic epithelium, where the proliferating cells in the crypts of the villi did not express CCN5, however in non-proliferating cells in the villi tips CCN5 levels were high. In contrast to the situation observed for vascular SMC, in which CCN2 is expressed in proliferating but not quiescent cells and CCN5 is expressed in the opposite expression pattern (Fan et al, 2000; Lake et al, 2003), both CCN2 and CCN5 were expressed in a similar pattern in the colonic epithelium. This suggests that although CCN5 and CCN2 both are needed for cell growth control, they are not necessarily regulated in a complementary fashion in all cell types.

In the kidney and urogenital system, the terminal buds of kidney mesonephric tubules express either CCN2 or CCN5, but not both, therefore these proteins may work in concert to regulate terminal bud proliferation and branching. The differential expression of CCN5 and CCN2 in early kidney development suggests that these two proteins may have opposite functions in stimulating or inhibiting branching in this system. The lack of CCN5 in the endothelial cells of the glomerular tufts suggests that CCN5 has a different function in the ECM of larger vessels with more tightly coupled endothelial cells compared to those of the more permeable glomerular tufts. CCN2 mRNA and protein expression has been reported to be low but present in healthy adult kidney (Riser et al, 2005).

CCN5 has been observed to be expressed in secretory structures, including in the endometrial glands (Mason et al, 2004a), consistent with CCN5 protein found herein in most secretory glands including the exocrine pancreas and thyroid. CCN5 is expressed in many steroid-secreting cells during human development, including human testicular Leydig cells and the cortisol-secreting cells of the adrenal glands. The low expression levels of CCN5 in fetal uterine smooth muscle and glands was surprising, because it is expressed at high levels in adult uterine tissue both in human and rat. (Mason et al, 2004a; Mason et al, 2004b). Although CCN5 is highly expressed in response to high estrogen levels in the adult, this tissue may not have the ability to regulate CCN5 in response to estrogen levels until sexual maturity is reached.

In most cell and tissue types, CCN5 expression was observed primarily in the cytoplasm and periphery of cells as previously observed in cultured vascular SMC (Lake et al, 2003). Nuclear localization of CCN5 was also observed herein in some cells. Nuclear localization was particularly prominent in the cells of the spinal cord. The variation in subcellular localization of CCN5 among different cell types supports the idea that CCN5 may subserve several different functions within the cell.

The surprisingly broad expression pattern of CCN5 in most embryonic and fetal tissues, combined with its increasingly tissue-specific expression with developmental age, indicates a cell and tissue-specific set of functions for CCN5. Examples herein and the work of others (Gellhaus et al, 2004; Leask and Abraham, 2006; Planque et al, 2006) observe CCN5 expression on the cell periphery, in the cytoplasm, and in the nucleus. These findings point to a complex set of conditions and parameters that regulate CCN5 and its biological roles, including growth state, hormonal influences, extracellular milieu, cell cycle position, reproductive status, developmental stage, contact/density, cytokine exposure, adhesion, disease, and other influences.

### Example 24

CCN5 Distribution in Adult Tissues

To further describe the biological functions of the CCN5 protein, it is necessary to determine its normal protein distribution in vivo. The earlier studies of CCN5 expression patterns are based largely on Northern blot analyses of RNA samples prepared from isolated vertebrate tissues. Because CCN proteins are secreted, it is possible that the pattern of mRNA expression would not match that of protein distribution. Thus, in order to fully describe CCN5 expression patterns, CCN5 protein distribution in diverse rodent tissues was examined using immunohistochemistry. Expected results included that (1): CCN5 protein would be found mostly in smooth muscle, glandular epithelium, and endothelial cells, and only slightly in:other tissues. (2): CCN2 protein expression would be highest in endothelial cells, bone, cartilage, and connective tissue, and much lower in other tissues. (3): Because the biological effects of CCN5 expression have been observed to be opposite to that of CCN2, the two proteins would have opposite and non-overlapping patterns of expression. Surprisingly, the immunohistochemistry results demonstrated that: (1) both CCN2 and CCN5 are widely distributed in all tissues tested in both rat and mouse, (2) CCN2 and CCN5 have overlapping and very similar patterns of distribution, and (3) many cell types demonstrate intra-nuclear localization of both CCN5 and CCN2.

### Adult Animals and Tissue Samples

Organs were carefully dissected out immediately post-mortem. They were cut into pieces small enough to allow complete fixation of the entire sample via immersion. Organ/tissue samples were rinsed in cold phosphate-buffered saline (PBS) and placed in 4% paraformaldehyde in PBS overnight at 4° C. The fixed tissue samples were transferred to 30% sucrose in PBS for 20 hrs and then frozen in embedding medium (OCT; Tissue-Tek) using liquid nitrogen. Blocks of frozen tissue were stored at −70° C. until sectioning (7-20 μM) using a Leica CM3050S cryostat and glass slides (Superfrost/Plus; Fisher). All slides were stored at −20° C. until further analysis.

### Immunohistochemistry

Immunohistochemical analysis was performed using the Vectastain Elite ABC kit (PK 6101; Vector Laboratories; Burlingame, Calif.). Slides were incubated with anti-CCN5 (1:500 in PBS; affinity purified polyclonal antibody to a rat peptide consisting of amino acids 103-117 of the CCN5 VWC domain) and anti-CCN2 (1:500 in PBS, affinity-purified polyclonal antibody to a mouse peptide that includes amino acids 223-348 of the CCN2 protein; ab6992; Abcam, Inc.; Cambridge, Mass.) overnight at 4° C. The specificity of the anti-CCN5 antibody was verified by Western blot analysis of cultured cell and tissue lysates (prominent 28 kDa band produced in rat smooth muscle cells transfected with a recombinant adenovirus that included the full-length rat CCN5 cDNA sequence). A second anti-CCN5 antibody against a rat peptide in the IGF-BP domain produced the same Western blot results as our antibody. After incubation with the secondary antibody (biotinylated goat anti-rabbit IgG) and the ABC reagent (30 minutes each at room temperature), and a 5-minute staining with the DAB peroxidase substrate reagent (SK 4100; Vector Laboratories; Burlingame, Calif.), the slides were counterstained with Harris modified hematoxylin with acetic acid (Fisher) for 1 minute. All slides were dehydrated and embedded in permanent mounting medium (#13510; DPX Mountant; Electron Microscopy Sciences; Hatfield, Pa.) and photographed using a microscope (Zeiss Axioscope) and a digital camera system (SPOT; Diagnostic Instruments).

### Results

Immunohistochemistry analysis revealed CCN5 and CCN2 staining in numerous cell types of most tissues and organs analyzed, in both mouse and rat. The staining patterns of mouse tissue samples stained with anti-CCN5 were generally very similar to CCN2 patterns (data collected from photomicrographs of stained slides and summarized in Tables 2-3 on pp. 75-76 herein). Rat tissues had very similar patterns of CCN5 and CCN2 staining.

High levels of CCN5 protein were detected in rat arterial smooth muscle and endothelium and in cultured aortic smooth muscle cells as previously described (Lake et al., 2003). Using the more sensitive DAB-horseradish peroxidase method employed herein, we have refined the localization of CCN5 in the walls of the aorta and other large arteries was refined to reveal particularly intense expression in the innermost portion of the media and in the endothelium. Adventitia also displays significant CCN5 expression. CCN2 expression was detected Immunohistochemical analysis of mouse heart tissue revealed similar patterns of both CCN2 and CCN5. Both proteins were found in the cytoplasm of the ventricular myocardium, with the highest levels in cardiac myocytes close to the epicardium. Higher levels of both CCN2 and CCN5 were found in the atria and in the valves. Rat heart sections revealed the same pattern of CCN2 and CCN5 as that of the mouse.

The CNN5 and CNN2 contents of a large number of other adult tissues were determined, and the details of the results obtained are summarized in Tables 2 and 3 for each of mouse and rat, respectively. CCN5 and CCN2 proteins were detected in all organs and tissues examined in both mouse and rat. Immunohistochemistry results for both mouse and rat tissues varied in distribution and intensity among the different cell types in each tissue tested (Tables 2-3). Both proteins were most commonly observed in the cytoplasm, varying in intensity from very little (spleen) to very intense (stomach). The lowest levels of CCN2 and CCN5 among all the tissues analyzed were observed in the spleen. In many tissues (lung, stomach, duodenum, liver, kidney, ovary, thymus, olfactory epithelium, and brain), nuclear staining was observed for both CCN2 and CCN5 (Tables 2-3). In almost all tissues, the intensity and distribution of CCN5 resembled that of CCN2, except for small differences noted above in the rat liver, kidney, and brain (Tables 2-3).

From earlier published work it was expected that CCN5 protein would be present mostly in smooth muscle, glandular epithelium, and endothelial cells, and not in other tissues of the adult. Instead, examples herein show that CCN5 was found widely distributed throughout all tissues of the mouse and rat. Since the biological effects of CCN2 and CCN5 have been demonstrated to be opposite in many assays, it was further expected that the distributions of both proteins would be largely non-overlapping and complementary. Instead, both proteins had very similar patterns of tissue distribution, with only small differences in levels of expression as described above. It was further expected that CCN2 and CCN5 would localize only in the cytoplasm or on cell surfaces. Instead, both proteins were found to be present in cell nuclei of numerous tissues.

Studies previously demonstrated the presence of CCN5 protein in rat uterine smooth muscle and endometrial glandular epithelium (Mason et al., 2004) and in rat arterial smooth muscle (Lake et al., 2003). CCN5 has also been described in human fetal bone (Kumar et al., 1999); human pancreas (Dhar et al., 2007); and breast ductal and lobular epithelium (Banerjee et al., 2003). CCN5 protein and mRNA has also been detected in numerous cultured cells and tissue specimens using Western and Northern blots, respectively (summarized in Gray and Castellot, 2004). The results of these earlier screening studies were however extremely variable and inconsistent, with an excess of negative findings in some studies. Therefore the present comprehensive analysis for CCN5 protein in adult vertebrate tissues was undertaken.

The adult tissue CCN2 and CCN5 protein expression patterns are similar to those observed in tissues of late gestation mouse embryos (Malmquist et al., 2007 incorporated herein by reference in entirety). The present immunohistochemistry results have been further corroborated by Western blot analyses of mouse and rat tissue protein lysates, and in immunofluorescent microscopic analysis of cultured cells prepared from many of the tissue types analyzed.

Widespread expression of both CCN5 and CCN2 strongly suggests that both proteins have numerous functions, most of which remain uncharacterized. Since both CCN2 and CCN5 are expressed in similar patterns, these proteins might be coordinately regulated, for example, by the same ligands and/or intracellular signaling pathways in various cell types. Since CCN5 and CCN2 frequently co-localize, these proteins further might modulate the effects of each other at the physical level by competing for or interfering with the same receptors. CCN5 might be necessary for all cell types because it is present in all cell types and its gene is non-redundant, in contrast to the other 4-domain CCN proteins.

TABLE 2
Summary of mouse tissue CCN5 and CCN2 expression

| Tissue | CCN2 | CCN5 |
|---|---|---|
| aorta | moderate in endothelial cells | moderate-high in endothelial |
| | and adventitia; weak in media | cells and adventitia: high in |
| | | inner media |
| duodenum | moderate; cytoplasmic-all cell | moderate; cytoplasmic-all cell |
| | types; nuclear-Brunner's | types; nuclear-Brunner's |
| | gland and villi cells | gland and villi cells |
| fallopian | moderate in cytoplasm of all | moderate in cytoplasm of all |
| tube | cell types; moderate in some | cell types; moderate in some |
| | nuclei of stroma | nuclei of stroma |
| heart | weak cytoplasmic; at | moderate cytoplasmic, at |
| | myocardial edges, atria, and | myocardial edges, atria, and |
| | valves | valves |
| kidney | moderate cytoplasmic and | strong cytoplasmic and |
| | nuclear-all cell types except | nuclear-all cell types except |
| | glomerular cells | glomerular cells |
| liver | very weak cytoplasmic; | weak cytoplasmic; stronger in |
| | stronger in hepatocytes at | hepatocytes at edges |
| | edges | |
| lung | moderate; cytoplasmic; all cell | moderate; cytoplasmic; all cell |
| | types; some nuclei | types; some nuclei |
| olfactory | weak in cytoplasm and some | weak in cytoplasm and a few |
| | nuclei of epithelial cells; | nuclei of epithelial cells; |
| | moderate in cytoplasm and | moderate in cytoplasm and |
| | some nuclei of lamina propria; | some nuclei of lamina propria; |
| ovary | moderate cytoplasmic and | moderate cytoplasmic and |
| | some nuclear-all cell types | some nuclear-all cell types |
| pancreas | all cytoplasmic-weak in | all cytoplasmic-weak in |
| | endocrine cells; moderate in | endocrine cells; moderate in |
| | exocrine cells | exocrine cells |
| skeletal | moderate cytoplasmic, | moderate cytoplasmic, |
| muscle | moderate near edges | moderate near edges |
| spleen | very weak cytoplasmic in all | very weak cytoplasmic in all |
| | cells | cells |
| stomach | strong; cytoplasmic-all cell | strong; cytoplasmic-all cell |
| | types; nuclear-tubular gland | types; nuclear-tubular gland |
| | cells | cells |
| uterus | moderate in cytoplasm of all | moderate in cytoplasm of all |
| | cell types; moderate in some | cell types; moderate in some |
| | nuclei of stroma | nuclei of stroma |

TABLE 3
Summary of rat tissue CCN5 and CCN2 expression.

| Tissue | CCN2 | CCN5 |
|---|---|---|
| aorta | moderate in endothelial cells | Moderate-high in endothelial |
| | and adventitia; weak in media | cells and adventitia; high in |
| | | inner media |
| brain | weak in cytoplasm in most | moderate in cytoplasm in |
| | cells; strong in over half of all | most cells; strong inover half |
| | nuclei | of all nuclei |
| fallopian | moderate in cytoplasm of all | moderate in cytoplasm of all |
| tube | cell types; moderate in some | cell types; moderate in some |
| | nuclei of stroma | nuclei of stroma |
| heart | cytoplasmic staining on edges | weak cytoplasmic staining, |
| | only | stronger at edges |
| kidney | moderate cytoplasmic and | strong cytoplasmic and |
| | nuclear-all cell types except | nuclear-all cell types except |
| | glomerular cells | glomerular cells |
| liver | very weak cytoplasmic; | weak cytoplasmic; stronger in |
| | stronger in hepatocytes at | hepatocytes at edges |
| | edges | |
| lung | moderate; cytoplasmic; all cell | moderate; cytoplasmic; all cell |
| | types; some nuclei | types; some nuclei |
| ovary | moderate cytoplasmic and | moderate cytoplasmic and |
| | some nuclear-all cell types | some nuclear-all cell types |
| skeletal | moderate cytoplasmic, | moderate cytoplasmic, |
| muscle | moderate near edges | moderate near edges |
| spleen | very weak cytoplasmic in all | very weak cytoplasmic in all |
| | cells | cells |
| uterus | moderate in cytoplasm of all | moderate in cytoplasm of all |
| | cell types; moderate in some | cell types; moderate in some |
| | nuclei of stroma | nuclei of stroma |

### REFERENCES

Ando H, Fukuda N, Kotani M, Yokoyama Si, Kunimoto S, Matsumoto K, Saito S, Kanmatsuse K, Mugishima H. (2004). European Journal of Pharmacology, 483: 207-214.
Babic A M, Chen C C, Lau L F. (1999). Cell Biol 19:2958-66.
Baelder R, Fuchs B, Bautsch W, Zwirner J, Kohl J, Hoymann H G, Glaab T, Erpenbeck V, Krug N, Braun A. (2005). J Immunol 174:783-9.
Ball D K, Surveyor G A, Diehl J R, Steffen C L, Uzumcu M, Mirando M A, Brigstock D R. (1998). BiolReprod, 59:828-835.
Banerjee S, Saxena N, Sengupta K, Tawfik O, Mayor M S, and Banerjee S K. (2003). Neoplasia 5 (1): 63-73
Beddy D, Mulsow J, Watson R W G, Fitzpatrick J M, and O'Connell P R. (2006). British Journal of Surgery 93 (10): 1290-1296
Bennett M R, O'Sullivan M. (2001). Pharmacology & Therapeutics, 91:149-166.
Bentzon J F, Weile C, Sondergaard C S, Hindkjaer J, Kassem M, Falk E. (2006). Arteriosclerosis, Thrombosis & Vascular Biology, 26:2696-2702.
Bradham D M, Igarashi A, Potter R L, Grotendorst G R. (1991). J Cell Biol 114:1285-94.
Brightling C E, Bradiing P, Symon F A, Holgate S T, Wardlaw A J, Pavord D M. (2002). N Engl J Med 346:1699-1705.
Brigstock D R, Lau L, Perbal B. (2005). J Clin Pathol 58:463-5.
Brigstock D R. (2003). J Endocr 178:169-75.
Burgess J K. (2005). Clinical and Experimental Pharmacology and Physiology 32 (11): 988-994
Busse W, Elias J, Sheppard D, Banks-Schlegel S. (1999). Am J Respir Crit Care Med 160:1035-42.
Busse W W, Lemanske RF Jr. (2001). N Engl J Med 344:350-62.
Candido R, Forbes J M, Thomas M C, Thallas V, Dean R G, Burns W C, Tikellis C, Ritchie R H, Twigg S M, Cooper M E, and Burrell L M. (2003). Circulation Research 92 (7): 785-792
Carulli M T, Ong V H, Ponticos M, Shiwen X, Abraham D J, Black C M, and Denton C P. (2005). Arthritis and Rheumatism 52 (12):3772-3782
Castellot J J Jr, Addonizio M L, Rosenberg R, Karnovsky M J (1981). J Cell Biol 90:372-9.
Cataldo D D, Gueders M M, Rocks N, Sounni N E, Evrard B, Bartsch P, Louis R, Noel A, Foidart J M. (2003). Cell Mol Biol 49:875-84.
Centers for Disease Control and Prevention. (1995). Asthma; United States, 1982-1992 JAMA, 273:451-2.
Cervello M, Giannitrapani L, Labbozzetta M, Notarbartolo M, D'Alessandro N, Lampiasi N, Azzolina A, and Montalto G. (2004). Signal Transduction and Communication in Cancer Cells 1028: 432-439
Charonis A, Tsilibary P, Kramer R, Wissig S. (1983). Microvascular Research, 26:108-115.
Chien W. Yin D, Gui D, Mori A, Frank J M, Said J, Kusuanco D, Marchevsky A, McKenna R, Koeffler H P. (2006). Mol Cancer Res 4:1-8.
Cicha I, Yilmaz A, Klein M, Raithel D, Brigstock D R, Daniel W G, Goppelt-Struebe M, Garlichs CD. (2005). Arteriosclerosis, Thrombosis & Vascular Biology, 25:1008-1013.
Corbel M, Caulet-Maugendre S, Germain N, Lagente V, Boichot E. (2003). Clin Exp Allergy 33:696-704.
De Falco M, Staibano S, D'Armiento F P, Mascolo M, Salvatore G, Busiello A, Carbone I F, Pollio F, and Di Lieto A. (2006). Journal of the Society for Gynecologic Investigation 13 (4): 297-303
Dean R G, Balding L C, Candido R, Burns W C, Cao Z M, Twigg S M, and Burrell L M. (2005). Journal of Histochemistry & Cytochemistry 53 (10): 1245-1256
Delmolino L M, Stearns N A, Castellot J J Jr (1997). Mol Biol Cell 8:287a.
Delmolino L M, Stearns N A, Castellot J J Jr (2001). J Cell Physiol 188:45-55.
Deshpande D A, White T A, Guedes A G P, Milla C, Walseth T F, Lund F E, Kannan M S. (2005). Am J Resp Cell Molec Biol 32:149-156.
Dhar G, Mehta S, Banerjee S, Gardner A, McCarty B M, Mathur S C, Campbell D R, Kambhampati S, Banerjee S K. (2007). Loss of WISP-2/CCN5 signaling in human pancreatic cancer: a potential mechanism for epithelial/mesenchymal-transition. Cancer Let Epub March 2007.
Dzierzak E and Medvinsky A. (1995). Trends in Genetics 11 (9): 359-366
Elias J A, Lee C G, Zheng T, Ma B, Homer R J, Zhu Z. (2003). J Clin Invest 111:291-7.
Ellis P D, Chen Q, Barker P J, Metcalfe J C, Kemp P R (2000a). Arteriosclerosis, Thrombosis & Vascular Biology, 20:1912-1919.
Ellis P D, Chen Q, Barker P J, Metcalfe J C, Kemp P R (2000b): Arterioscler Thromb Vasc Biol, 20:1912-1919.
Evans K L J, Bond R A, Corry D B, Shardonofsky F R. (2003). J Appl Physiol 94:245-52.
Evans M J, Van Winkle L S, Fanucchi M V, and Plopper C G. (1999). American Journal of Respiratory Cell and Molecular Biology 21 (6): 655-657
Fan W H, Karnovsky M J (2002). Journal of Biological Chemistry, 277:9800-9805.
Fan W H, Pech M, and Karnovsky M J. (2000). European Journal of Cell Biology 79 (12): 915-923
Finckenberg P, Inkinen K, Ahonen J, Merasto S, Louhelainen M, Vapaatalo H, Muller D, Ganten D, Luft F, and Mervaala E. (2003). American Journal of Pathology 163 (1): 355-366
Forte A, Cipollaro M, Cascino A, Galderisi U (2007). Histology & Histopathology, 22:547-557.
Frazier K, Williams S, Kothapalli D, Klapper H, Grotendorst G R (1996). JInvest Dermatol, 107:404-411.
French D M, Kaul R J, D'Souza A L, Crowley C W, Bao M, Frantz G D, Filvaroff E H, and Desnoyers L. (2004). American Journal of Pathology 165 (3): 855-867
Friedrichsen S, Heuer H, Christ S, Cuthill D, Bauer K, Raivich G. (2005). Growth Factors 23:43-53.
Friedrichsen S, Heuer H, Christ S, Winckler M, Brauer D, Bauer K, and Raivich G. (2003). Cell and Tissue Research 312 (2): 175-188
Fritah A, Redeuilh G, and Sabbah M. (2006). Journal of Endocrinology 191 (3): 613-624
Fukunaga T, Yamashiro T, Oya S, Takeshita N, Takigawa M, Takano-Yamamoto T. (2003). Bone 33: 911-18.
Fukunaga-Kalabis M, Martinez G, Liu Z J, Kalabis J, Mrass P, Weninger W, Firth S M, Planque N, Perbal B, Herlyn M (2006). J Cell Biol, 175:563-569.
Fukutomi T, Zhou Y H, Kawai S, Eguchi H, Wands J R, and Li J S. (2005). Hepatology 41 (5): 1096-1105
Gao R, Ball D K, Perbal B, Brigstock D R. (2004). J Hepatol 40:431-8.
Gautam A, Densmore C L, Golunski E, Xu B, Waldrep J C. (2001). Molec Therapy 3:551-6.
Gawronska-Kozak B (2004). Tissue Engineering, 10:1251-1265.
Gellhaus A, Dong X, Propson S, Maass K, Klein-Hitpass L, Kibschull M, Traub O, Willecke K, Perbal B, Lye S J, and Winterhager E. (2004). Journal of Biological Chemistry 279 (35): 36931-36942
Glaab T, Ziegert M, Baelder R, Korolewitz R, Braun A, Hohifeld J M, Mitzner W,. Krug N, Hoymann H G (2005). Respir Res 6:139-48.
Gray M R and Castellot J J. (2005). Function and Regulation of CCN5. In: Perbal, B V and Takigawa, M. (Eds) CCN Proteins: A New Family of Cell Growth and Differentiation Regulators. Imperial College Press, London, pp. 207-238.
Gray M R, Castellot J J. (2004). Function and Regulation of CCN5, (Ch. 12) in CCN Proteins: A New Family of Cell Growth and Differentiation Regulators, Imperial College Press, London.
Gray M R, Castellot J J. (2005). (Ch. 2) in Uterine Fibroids. Pathogenesis and Management, Taylor and Francis, London.
Gray M R, Malmquist J A, Sullivan M, Blea M, Castellot J J: (2007). Cell Commun Signal 1 (2): 145-158
Grotendorst G R, Duncan M R. (2005). Faseb J, 19:729-738.
Grzeszkiewicz T M, Lindner V, Chen N, Lam S C, Lau L F. (2002). Endocrinology, 143:1441-1450.
Harlow C R, Davidson L, Bums K H, Yan C, Matzuk M M, Hillier S G. (2002). Endocrinol 143: 3316-25.
Haydont V, Mathe D, Bourgier C, Abdelali J, Aigueperse J, Bourhis J, and Vozenin-Brotons M C. (2005). Radiotherapy and Oncology 76 (2): 219-225
Hilfiker A, Hilfiker-Kleiner D, Fuchs M, Kaminski K, Lichtenberg A, Rothkotter H-J, Schieffer B, Drexler H. (2002). Circulation, 106:254-260.
Hirasaki S, Koide N, Ujike K, Shinji T, and Tsuji T. (2001). Hepatology Research 19 (3): 294-305
Hirsch E, Gullberg D, Balzac F, Altruda F, Silengo L, and Tarone G. (1994). Developmental Dynamics 201 (2): 108-120
Homer R J, Elias J A. (2000). Clin Chest Med 21:331-43.
Hoofnagle M H, Thomas J A, Wamhoff B R, Owens G K. (2006). Arteriosclerosis, Thrombosis & Vascular Biology, 26:2579-2581.
Inadera H, Hashimoto S, Dong H Y, Suzuki T, Nagai S, Yamashita T, Toyoda N, and Matsushima K. (2000). Biochemical and Biophysical Research Communications 275 (1): 108-114
Ito Y, Goldschmeding R, Bende RJ, Claessen N, Chand M A, Kleij L, Rabelink T J, Weening J J, Aten J. (2001). J Am Soc Nephrol 12: 472-84.
Ivkovic S, Yoon B S, Popoff S N, Safadi F F, Libuda D E, Stephenson R C, Daluiski A, and Lyons K M. (2003). Development 130 (12): 2779-2791
Johnson J R, Wiley R E, Fattouh R, Swirski F K, Gajewska B U, Coyle A J, Gutierrez-Ramos J-C, Ellis R,. Inman M D, Jordana M. (2004). Am J Respir Crit Care Med 169:378-85.
Jones J A, Gray M R, Oliveira B E, Koch M, Castellot J J: (2007). Cell Commun Signal 1 (2): 127-143.
Kauffman M H. (1992) Atlas of Mouse Development. Elsevier Ltd., San Diego, Calif.
Kireeva M L, Latinkic B V, Kolesnikova T V, Chen C C, Yang G P, Abler A S, Lau L F. (1997). Experimental Cell Research, 233:63-77.
Kocialkowski S, Yeger H, Kingdom J, Perbal B, and Schofield P N. (2001). Anatomy and Embryology 203 (6):417-427
Kuhn C, Homer R J, Zhu Z, Ward N, Flavell R A, Geba G P, Elias J A. (2000). Am J Respir Cell Mol Biol 22:289-295.
Kumar A, Lindner V. (1997). Arterioscler Thromb Vasc Biol 17:2238-44.
Kumar S, Hand A T, Connor J R, Dodds R A, Ryan P J, Trill J J, Fisher S M, Nuttall M E, Lipshutz D B, Zou C, et al. (1999). Journal of Biological Chemistry, 274:17123-17131.
Kuo K-H, Seow C Y. (2003). J Cell Sc 117:1503-11.
Kusser K L, Randall T D. (2003). Journal of Histochemistry & Cytochemistry, 51:5-14.
Lake A C, Bialik A, Walsh K, Castellot J J Jr. (2003). Am J Pathol 162:219-31.
Lake A C, Castellot J J Jr. (2003). Cell Commun Signal 1:5-17.
Lau L F and Lam S C-T. (1999). Experimental Cell Research 248 (1): 44-57
Lau L M and Lam S C-T. (2005). Integrin-Mediated CCN Functions. In: Perbal, B V and Takigawa, M. (Eds) CCN Proteins: A New Family of Cell Growth and Differentiation Regulators. Imperial College Press, London, pp. 61-80.
Leask A, Abraham D J. (2006). Journal of Cell Science, 119:4803-4810.
Leask A. (2004). Keio J Med 53:74-7.
Li GM, Xie Q, Shi Y, Li D G, Zhang M J, Jiang S, Zhou H J, Lu H M, and Jin Y X. (2006). Journal of Gene Medicine 8 (7): 889-900
Lopes S M C D, Feijen A, Korving J, Korchynskyi O. Larsson J, Karlsson S, Ten Dijke P, Lyons K M, Goldschmeding R, Doevendans P, and Mummery C L. (2004). Developmental Dynamics 231 (3): 542-550
Lyons-Giordano B, Conaway H, Kefalides N A. (1987). BiochemBiophysResComm, 148:1264-1269.
Maillard L, Van Belle E, Tio F O, Rivard A, Kearney M, Branellec D, Steg P G, Isner J M, Walsh K. (2000). Gene Therapy, 7:1353-1361.
Maisi P, Prikk K, Sepper R, Pirila E, Salo T, Hietanen J, Sorsa T. (2002). Acta Pathol Microbiol Immunol Scand 110:771-82.
Malmquist J A (now Jones J A), Gray M R, Oliveira B E, Koch M, Castellot J J: (2007). Cell Commun Signal 1 (2): 127-143.
Manara M C, Perbal B, Benini S, Strammiello R, Cerisano V, Perdichizzi S, Serra M, Astolfi A, Bertoni F, Alami J, Yeger H, Picci P, and Scotlandi K. (2002). American Journal of Pathology 160 (3):849-859
Manns J M, Uknis A B, Rico M C, Agelan A, Castaneda J, Arango I, Barbe M F, Safadi F F, Popoff S N, and DeLa Cadena R A. (2006). Arthritis and Rheumatism 54 (8): 2415-2422
Mason H R, Castellot J J, and Nowak R A. (2002). Molecular Biology of the Cell 13: 289A-289A
Mason H R, Grove-Strawser D, Rubin B S, Nowak R A, and Castellot J J. (2004a). Endocrinology 145 (2): 976-982
Mason H R, Lake A C, Wubben J E, Nowak R A, Castellot J J Jr. (2004b). Mol Hum Reprod 10 (3): 181-7.
Mason H R, Nowak R A, Morton C C, Castellot J J Jr. (2003). Am J Pathol 162:1895-1904.
McCormac J T and Greenwal G S. (1974). Journal of Endocrinology 62 (1): 101-107
Mishra-Gorur K, Delmolino L M, Castellot J J, Jr. (1998). Trends Glycosci Glycotechnol 10:193-210.
Mitra A K, Agrawal D K.(2006). Pharmacogenomics, 7:1185-1198.
Moussad E E A, Brigstock D R. (2000). Mol Genet Metab 71:276-92.
Natarajan D, Andermarcher E, Schofield P N, and Boulter C A. (2000). Developmental Dynamics 219 (3): 417-423
O'Brien T P and Lau L F. (1992). Cell Growth & Differentiation 3 (9): 645-654
Pennica D, Swanson T A, Welsh J W, Roy M A, Lawrence D A, Lee J, Brush J. Taneyhill L A, Deuel B, Lew M, et al. (1998). Proceedings of the National Academy of Sciences of the United States of America, 95 (25):14717-14722.
Perbal B V, Takigawa M (Eds). (2005a). CCN Proteins: A New Family of Cell Growth and Differentiation Regulators. London: Imperial College Press.
Perbal B V, Takigawa M. (2005b): The CCN Family of Proteins: An Overview. In: Perbal B V, Takigawa M (Eds) CCN Proteins: A New Family of Cell Growth and Differentiation Regulators. London: Imperial College Press: 1-18.
Perbal B. (2004) CNN proteins: multifunctional signaling regulators, Lancet 363 (9402):62-64.
Pires N M M, Jukema J W, Daemen M J A P, Quax P H A. (2006). Vascular Pharmacology 44:257-264.
Planque N, Li C L, Saule S, Bleau A M, and Perbal B. (2006). Journal of Cellular Biochemistry 99 (1): 105-116
Planque N. (2006). Cell Communication and Signaling 4: 7-25
Rachfal A W, Brigstock D R. (2005). Vitamins and Hormones, 70:69-103.
Rachfal A W, Luquette M H, Brigstock D R. (2004). J Clin Path 57:422-5.
Rajagopal V, Rockson S G. (2003). American Journal of Medicine 115:547-553.
Razzaque M S, Foster C S, and Ahmed A R. (2003). Investigative Ophthalmology & Visual Science 44 (5): 1998-2003
Riser B, Karoor S, and Peterson D. (2005) CCN Genes and the Kidney. In: Perbal, B V and Takigawa, M. (Eds) CCN Proteins: A New Family of Cell Growth and Differentiation Regulators. Imperial College Press, London, pp. 95-116.
Safadi F F, Xu J, Smock S L, Kanaan R A, Selimf A H, Odgren P R, Marks S C, Owen T A, and Popoff SN. (2003). Journal of Cellular Physiology 196 (1): 51-62
Saku T, Furthmayr H. (1989). J Biol Chem 264:3514-3523.
Schillinger M, Minar E. (2005). Vascular Health & Risk Management, 1:73-78.
Schober J M, Chen N, Grzeszkiewicz T M, Jovanovic I, Emeson E E, Ugarova T P, Ye R D, Lau L F, Lam S C T. (2002). Blood 99:4457-4465.
Schutze N, Noth U, Schneidereit J, Hendrich C, and Jakob F. (2005). Cell Commun Signal 3 (1): 5
Scott N A. (2006). Advanced Drug Delivery Reviews 58:358-376.
Shariatmadari R, Sipila P P, Huhtaniemi I T, Poutanen M. (2001). Biotechniques 30:1282-1285.
Shin J Y, Hur W, Wang J S, Jang J W, Kim C W, Bae S H, Jang S K, Yang S H, Sung Y C, Kwon O J, and Yoon S K. (2005). Experimental and Molecular Medicine 37 (2): 138-145
Smith R C, Walsh K. (2001). Acta Physiologica Scandinavica 173:93-102.
Stearns N A, Prigent-Richard S, Letourneur D, Castellot J J, Jr. (1997). Anal Biochem 247:348-356.
Surveyor G A and Brigstock D R. (1999). Growth Factors 17 (2): 115-124
Surveyor G A, Wilson A K, and Brigstock D R. (1998). Biology of Reproduction 59 (5): 1207-1213
Takigawa M, Nakanishi T, Kubota S, and Nishida T. (2003). Journal of Cellular Physiology 194 (3): 256-266
Takigawa M, Nishida T, and Kubota S. (2005). Roles of CCN2/CTGF in the Control of Growth and Regeneration. In: Perbal, B V and Takigawa, M. (Eds) CCN Proteins: A New Family of Cell Growth and Differentiation Regulators. Imperial College Press, London, pp. 19-60.
Tan E M, Dodge G R, Sorger T, Kovalszky I, Unger G A, Yang L, Levine E M, lozzo R V. (1991). Laboratory Investigation 64:474-482.
Tanaka I, Morikawa M, Okuse T, Shirakawa M, and Imai K. (2005). Biochemical and Biophysical Research Communications 334 (4): 973-978
Tarone G, Hirsch E, Brancaccio M, De Acetis M, Barberis L, Balzac F, Retta F, Botta C, Altruda F, and Silengo L. (2000). International Journal of Developmental Biology 44 (6): 725-731
Tavian M and Peault B. (2005). Experimental Hematology 33 (9): 1062-1069
Tong Z Y, Brigstock D R. (2006). J Endocrinol 188:R1-8.
Toutouzas K, Colombo A, Stefanadis C. (2004). European Heart Journal 25:1679-1687.
Travis W E, Colby.T V, Koss M N, Rosado-de-Christenson M L, Muller N L, King T E, Jr. (2002). Non-plastic disorders of the lower respiratory tract, in Atlas of Non tumor Pathology 2:457-71.
Van Tuyl M and Post M. (2003). Molecular mechanisms of lung development and lung branching morphogenesis. In: Polin R A, Fox W W, and Abman S H. (Eds) Fetal and Neonatal Physiology (3rd ed.). Saunders, Harcourt Health Sciences, Philadelphia, Pa., pp. 812-821.
Volpe M V, Nielsen H C, Archavachotikul K, Ciccone T J, and Chinoy M R. (2003). Molecular Genetics and Metabolism 80 (1-2): 242-254
Vozenin-Brotons M C, Milliat F, Sabourin J C, de Gouville A C, Francois A, Lasser P, Morice P, Haie-Meder C, Lusinchi A, Antoun S, Bourhis J, Mathe D, Girinsky T, and Aigueperse J. (2003). International Journal of Radiation Oncology*Biology*Physics 56 (2): 561-572
Wahab N A, Brinkman H, Mason R M. (2001). J Biochem 359:89-97.
Wert S E. (2004) Normal and abnormal structural development of the lung. In: Polin R A, Fox W W, and Abman S H. (Eds) Fetal and neonatal physiology 3rd ed. W.B. Saunders Co., Philadelphia, Pa., pp. 783-793.
Willems E, Mateizel I, Kemp C, Cauffman G, Sermon K, and Leyns L. (2006). International Journal of Developmental Biology 50 (7): 627-635
Xu Q. (2004). American Journal of Pathology 165:1-10.
Yamaai T, Nakanishi T, Asano M, Nawachi K, Yoshimichi G, Ohyama K, Komori T, Sugimoto T, and Takigawa M. (2005). Journal of Bone and Mineral Metabolism 23 (4): 280-288
Zhang R, Averboukh L, Zhu W M, Zhang H, Jo H, Dempsey P J, Coffey R J, Pardee A B, Liang P. (1998). Molecular and Cellular Biology 18:6131-6141.

What is claimed is:

1. A method for treating a smooth muscle proliferation-based disorder in a mammalian subject, the method comprising expressing CCN5 in or administering CCN5 protein to smooth muscle cells, wherein the disorder is at least one selected from the group consisting of: asthma, vascular injury, persistent pulmonary hypertension in the newborn (PPNH), pulmonary hypertension in adults, megaureter, pyloric stenosis, uterine fibroids, lymphangioleiomyomatosis (LAM), cervical incompetence, and cancer.

2. The method according to claim 1, wherein the disorder is asthma, and the CCN5 is expressed in airway smooth muscle cells (ASM) by contacting ASM with an expression vector encoding CCN5, the vector selected from a virus vector and a nucleic acid vector, or contacting ASM with recombinantly produced CCN5 protein.

3. The method according to claim 1, wherein expression of CCN5 is mediated by a small molecule or other agent that causes CCN5 to be over-expressed.

4. The method according to claim 2, wherein the vector is delivered via inhalation.

5. The method according to claim 3, wherein the small molecule or other agent is delivered via inhalation.

6. A method for evaluating airway remodeling comprising: modulating the expression of CCN5 in ASM in a mammal having symptoms of chronic asthma; and assessing the morphology of ASM in comparison to a control having the symptoms and otherwise identical to the mammal and not modulated in expression of CCN5.

7. A method for treating a smooth muscle disorder in a mammal comprising reducing the expression or activity in ASM of one or more genes that are suppressed by expression of CCN5.

8. The method according to claim 7, wherein the one or more genes is selected the group consisting of LILRA1, DEFB103A, LOC387643, LY6K and OR4X2.

9. The method according to claim 8, wherein the expression of one or more genes selected from the group consisting of LILRA1, DEFB103A, LOC387643, LY6K and OR4X2 is reduced in a smooth muscle cell by expressing an antisense RNA, ribozyme or siRNA that targets RNA encoding one or more genes selected from the group consisting of LILRA1, DEFB103A, LOC387643, LY6K and OR4X2.

10. The method according to claim 8, wherein the expression or activity of one or more genes selected from the group consisting of LILRA1, DEFB103A, LOC387643, LY6K and OR4X2 is mediated by a small molecule or other agent that reduces the expression or activity of one or more genes selected from the group consisting of LILRA1, DEFB103A, LOC387643, LY6K and OR4X2.

11. The method according to claim 9, wherein expressing an antisense RNA, ribozyme or siRNA further comprises delivering a nucleic acid expression vector by inhalation.

12. The method according to claim 10, wherein the small molecule or other agent is delivered via inhalation.

13. The method according to claim 8, wherein reducing expression further comprises contacting a smooth muscle cell with an antisense oligonucleotide, ribozyme, or siRNA that targets one or more genes selected the group consisting of LILRA1, DEFB103A, LOC387643, LY6K and OR4X2.

14. The method according to claim 13, wherein contacting the cell with the antisense oligonucleotide, ribozyme, or siRNA further comprises delivering by inhalation.

15. The method according to claim 1, wherein the vascular injury is at least one condition selected from the group of tensile strain, shear strain, vessel rupture, intimal rupture, penetrating trauma, blunt trauma, transection, contusion, laceration, arteriovenous (AV) fistula formation, vessel spasm, external compression, mural contusion, thrombosis, and aneurysm formation.

16. The method according to claim 1, wherein the vascular injury is stenosis or restenosis associated with at least one of wire injury, ligation injury, arteriovenous shunt, coronary artery bypass graft, endarterectomy, hypertension and balloon angioplasty.

17. The method according to either of claims 15 or 16 wherein following expressing CCN5 in smooth muscle cells the method further comprises observing substantial inhibition of neointimal lesion formation in comparison to a vascular injury otherwise identical not expressing CCN5.

18. The method according to either of claims 15 or 16 wherein following expressing CCN5 in smooth muscle cells the method further comprises observing substantial inhibition of at least one cell parameter selected from the group of proliferation, motility and matrix metalloprotease production.

19. The method according to claim 1, wherein the disorder is vascular injury, and the CCN5 is expressed in vascular smooth muscle cells by contacting vascular smooth muscle cells with an expression vector encoding CCN5, the vector selected from a virus vector and a nucleic acid vector, or contacting the cells with recombinantly produced CCN5 protein.

20. The method according to claim 19, wherein contacting the cells with recombinantly produced CCN5 protein is delivery by catheter or by coated stent.

21. The method according to claim 1, wherein the vascular injury arises from atherosclerosis.

22. The method according to claim 1, wherein the disorder is cancer, the method further comprising expressing CCN5 in vascular smooth muscle cells of a tumor by contacting the tumor with an expression vector encoding CCN5, the vector selected from a virus vector and a nucleic acid vector, or contacting the cells with recombinantly produced CCN5 protein.

23. The method according to claim 22 further comprising expressing CCN5 in a tumor by contacting the tumor with an expression vector encoding CCN5, the vector selected from a virus vector and a nucleic acid vector, or contacting the tumor with recombinantly produced CCN5 protein.

24. The method according to claim 23, wherein contacting the tumor further comprises inserting a drug delivery device into the tumor, or surgically implanting a device into the tumor.

25. The method according to claim 1, wherein the cancer is a tumor.

26. The method according to claim 25, wherein the tumor is at least one selected from the group of myolipoma, cystic liver tumor, hepatic tumor, angiolipoleiomyoma, leiomyoma, and leiomyosarcoma.
